(19) United States
(12) Patent Application Publication
(10) Pub. No.: US 2011/0136738 A1
(43) Pub. Date: Jun. 9, 2011

(54) Alternatively Transcribed Genes Associated with Schizophrenia

(75) Inventors: Keith J. Murphy, Dublin (IE); Robert H. Ring, Yardley, PA (US); Sean Mulvany, Co. Meath (IE); David von Schack, Arlington, MA (US); Ciaran M. Regan, Dublin (IE)

(73) Assignee: University College Dublin, National University of Ireland, Dublin, Dublin 4 (IE)

(21) Appl. No.: 12/867,389

(22) PCT Filed: Feb. 11, 2009
(86) PCT No.: PCT/US09/33733

§ 371 Date: Feb. 17, 2011

Related U.S. Application Data
(60) Provisional application No. 61/029,209, filed on Feb. 15, 2008.

(51) Int. Cl. A61K 38/18; C40B 30/00; C07H 21/00; A61P 25/18

(52) U.S. Cl. 514/8.3; 506/7; 536/23.1

(57) ABSTRACT

Methods of identifying gene targets associated with schizophrenia or the symptoms thereof, as well as to specific nucleic acid molecules that have been discovered to be associated with schizophrenia are provided. Accordingly, the invention also relates to methods of modulating schizophrenia or the symptoms thereof, methods of diagnosing schizophrenia or the symptoms thereof, methods for predicting the susceptibility to schizophrenia or the symptoms thereof, and methods of identifying compounds that modulate schizophrenia or the symptoms thereof via manipulations of the nucleic acid molecules and their products.

### FIELD OF INVENTION

This invention relates generally to schizophrenia and related disorders, and to methods for detecting gene targets associated with these disorders, to methods of predicting susceptibility to schizophrenia, to and methods of diagnosing schizophrenia, to methods of treating schizophrenia, as well as to the gene targets themselves.

### BACKGROUND OF THE INVENTION

The term “schizophrenia” refers to a number of related disorders, and is characterized by a wide range of complex symptoms. Schizophrenia affects more than 1% of the population worldwide, and clinical symptoms include a constellation of positive symptoms (e.g., hallucinations, delusions, racing thoughts), negative symptoms (e.g., apathy, lack of emotion, poor or nonexistent social functioning), and cognitive symptoms (e.g., disorganized thoughts, difficulty concentrating and/or following instructions, difficulty completing tasks, learning and memory deficits). The illness usually develops between adolescence and age thirty. For some patients the disease is consistent and lifelong, whereas others may experience periodic episodes of psychosis.

Research in recent years has provided information necessary to understand some of the underlying neuropathology and etiology of schizophrenia, and has implicated dozens of genes, neurotransmitters, and neural microcircuits. Unlike other types of dementia, schizophrenia is not associated with visible neuropathological markers such as plaques, tangles, or Lewy bodies. The gliosis that is a marker of neuronal death in many neurodegenerative diseases is not present in schizophrenia. It has been suggested that the etiology and pathophysiology of schizophrenia are related to maturational or developmental brain processes such as the formation of neurites, synaptogenesis, neuronal pruning, or apoptosis.

Schizophrenia is thought to be the consequence of some combination of inherited genetic factors and external, non-genetic factors that affect the regulation and expression of genes controlling brain function, or that injure the brain directly. It is thought that the disease is likely polygenic with multiple susceptibility loci. Schizophrenia runs in families as indicated by twin and adoption studies which suggest that such familial aggregation is largely accounted for by genetic factors. These same studies, however, also suggest that familial genetic transmission can only account for some of the cases of schizophrenia. For example, the concordance rate in monozygotic twins is about forty percent, indicating that non-genetic factors must play a role in development of the disease. Further, schizophrenia persists despite the fact that the majority of individuals with the disease do not marry or procreate.

Research with schizophrenia models has revealed, for example, a possible role for dopamine, glutamate, and serotonin in the development of schizophrenia (see, e.g., Geyer et al., Psychopharmacology, 156, 117-154, 2001). Proposal for treatment of schizophrenia with dopamine or dopamine precursors has also been suggested (see, e.g., U.S. Pat. No. 7,115,256). In addition, many candidate genes have been identified from human postmortem brains using gene expression microarrays (see, e.g., Mimics et al., Trends in Neurosci., 24(8), 479-486, 2001; see also U.S. Pat. No. 7,220,581). Some of the genes identified have been proposed for use in diagnosis of schizophrenia (see U.S. Pat. No. 6,395,482).

Despite the wealth of information accumulated from years of research, the understanding of schizophrenia remains rudimentary. The neuropathological findings are controversial and not diagnostically useful, relevant genes have been difficult to identify, and treatment has not improved much over time. Current treatments have inadequate efficacy, and often are associated with intolerable side effects which can lead to discontinuation of treatment by patients.

There is therefore a strong need to identify genes involved in schizophrenia, genes that are useful as biomarkers for diagnosing schizophrenia, as well as for methods that can detect such gene targets so they can be utilized in screening therapeutics, in diagnosing schizophrenia, and in developing treatments for individuals with schizophrenia. There is also a great need for new biomarkers and methods for detecting susceptibility to schizophrenia, as well as for preventing or following up development of the disease. Diagnostic tools could prove extremely useful, as early identification of subjects at risk of developing schizophrenia would enable early intervention and/or prophylactic treatment to be administered. A need therefore exists for new methods and reagents to more accurately and effectively diagnose and treat schizophrenia.

### SUMMARY OF THE INVENTION

As used herein, schizophrenia encompasses any of the many disorders that are characterized by psychosis as a core or fundamental feature. The term schizophrenia refers to schizophrenia, schizophreniform disorder, schizoaffective disorder, schizotypical disorder, schizoid personality disorder, schizotypical personality disorder, delusional disorder, brief psychotic disorder, shared psychotic disorder, psychotic disorder due to a general medical condition, substance-induced psychotic disorder, and psychotic disorder not otherwise specified, as defined in the DSM-IV, DSM-IV-TR, or any other diagnostic criteria. Further, as used in the invention, schizophrenia can refer to the different schizophrenia subtypes, including the paranoid type, the disorganized type, the catatonic type, the undifferentiated type, and the residual type, as well as the symptoms associated with these aspect of the disorders. All possible symptoms of these disorders are also within the scope of the invention, and are encompassed by the term, schizophrenia.

In one embodiment of the invention, methods are provided for identifying gene targets that are associated with schizophrenia or with the symptoms of schizophrenia. Animal models (using living animals) of schizophrenia are utilized, and initiated, and from tissue obtained from at least one of those animals, transcriptional regulation is assessed over time, relative to the onset of the schizophrenia model. Initiating an animal model refers to the onset of the manipulation that induces the schizophrenia model, whether it is a behavioral, biological, or genetic manipulation, drug administration, or any other manipulation which yields a model of schizophrenia. In some embodiments, it is useful to measure gene expression from animals at time points after, and, optionally, before the initiation of the model and to compare the gene expression from before and after initiation. In some embodiments, it is useful to compare the gene expression, whether from before or after the initiation of the model, or both, to gene expression at one or more time points from control animals, which are not subject to a schizophrenia model. Transcripts are then detected which are dysregulated in tissue from animals that are a model of schizophrenia. Any change in gene expression observed in the schizophrenia model, whether relative to other time points in the same model, relative to another schizophrenia model, or relative to the same time point or time points in control animals can be informative with respect to gene targets for schizophrenia or the symptoms of schizophrenia.

Systems for carrying out all of the methods described herein are also provided, and optionally comprise a computer system and related software. For example, as relates to the foregoing method, the system may comprise groups of mice in which an animal model of schizophrenia in living animals has been initiated, and a computer system comprising software, said computer system configured to assess transcriptional regulation in tissue over time in animals that are a model of schizophrenia, wherein the tissue is sampled one or more times after the initiation of the model and optionally one or more times prior to the initiation of the model; compare the transcriptional regulation from prior to initiation of the model with transcriptional regulation from after the initiation of the model, and/or with transcriptional regulation assessed from tissue in living animals not subject to a schizophrenia model; and detect a transcript that is dysregulated in tissue from animals that are a model of schizophrenia. Optionally, the computer system may output a result which is indicative of gene targets associated with schizophrenia or schizophrenia symptoms.

In some embodiments, transcriptional regulation in the animal model is compared with transcriptional regulation of genes that have previously been identified as associated with schizophrenia or the symptoms thereof. Matching the regulation with these genes with newly identified genes can be informative in identifying new transcripts or known genes which have not been previously identified as associated with schizophrenia or the symptoms of schizophrenia.

Any model of schizophrenia, whether pharmacological or non-pharmacological, can be used in accordance with the methods of the invention, though in preferred embodiments, the model is the isolation rearing model or the maternal deprivation model

In some embodiments, the methods for screening for schizophrenia targets relate to gene expression products and the changes observed with respect to those gene expression products.

In another embodiment of the invention, nucleic acid sequences identified according to the screening methods of the invention are provided. In yet another embodiment, the invention provides nucleic acid sequences that comprises at least SEQ ID NO:1 through SEQ ID NO:16.

In other embodiments, the invention relates to methods for predicting the susceptibility to schizophrenia or the symptoms of schizophrenia. According to the methods of this invention, an individual provides a biological sample, and from this sample, gene expression or the products of gene expression are measured. The gene or genes measured include those that have been identified as “pre-symptomatic genes,” described herein, which are associated with circumstances that exist prior to the onset of schizophrenia, and predict the onset of schizophrenia or symptoms associated with schizophrenia. When dysregulation of one or more of these genes is detected, it is informative of the likelihood that schizophrenia or its symptoms will be present in that individual.

In some embodiments, the pre-symptomatic genes have not been previously identified as being associated with schizophrenia, or as being predictive of the susceptibility to schizophrenia or the symptoms of schizophrenia. In another embodiment, the genes assessed are chosen from among interferon-induced protein, interferon regulatory factor 7, and PKR. In yet another embodiment, the genes assessed are chosen from one or more of SEQ ID NO: 11 through SEQ ID NO: 16.

In some embodiments, the individual providing the biological sample possesses at least one risk factor for schizophrenia. In other embodiments, the individual is asymptomatic, and in yet other embodiments, the individual presents with one or more symptoms of schizophrenia, but is not clinically diagnosed with schizophrenia.

In another embodiment, the invention provides methods for diagnosing schizophrenia or the symptoms of schizophrenia. An individual provides a biological sample, and from this sample, gene expression or the products of gene expression is measured. The genes measured include those that have been identified as “symptomatic genes,” described herein, which are associated with schizophrenia or the symptoms of schizophrenia. When dysregulation of one or more of these genes is detected, it is diagnostic of the presence of schizophrenia or its symptoms in that individual.

In some embodiments, the symptomatic genes have not been previously identified as being associated with schizophrenia, or the symptoms of schizophrenia. In another embodiment, the genes assessed are chosen from among SEQ ID NO:1 through SEQ ID NO: 16.

In some embodiments, the individual to be diagnosed presents with symptoms or other signs which aid in the diagnosis of schizophrenia. In other embodiments, the diagnosis is aided by other clinical, behavioral, or biological assessment tools.

In yet another embodiment of the invention, methods for preventing schizophrenia or the symptoms of schizophrenia are provided. The gene targets obtained according to the invention, or which are described herein are upregulated or down-regulated, or the products of the gene targets are increased or decreased. The nature of the dysregulation of the gene associated with the onset of schizophrenia will inform whether to upregulate or downregulate gene expression, or whether to increase or decrease gene expression products. In some embodiments the method for preventing schizophrenia or the symptoms of schizophrenia are performed in conjunction with other methods for preventing schizophrenia or its symptoms. In some embodiments, more than one gene or its expression product is modulated.

In yet another embodiment of the invention, methods for treating schizophrenia or the symptoms of schizophrenia are provided. The gene targets obtained according to the invention, or which are described herein are upregulated or down-regulated, or the products of the gene targets are increased or decreased. The nature of the dysregulation of the gene associated with schizophrenia will inform whether to upregulate or downregulate gene expression, or whether to increase or decrease gene expression products. In some embodiments the method for treating schizophrenia or the symptoms of schizophrenia are performed in conjunction with other treatments for schizophrenia or its symptoms, including any behavioral and drug therapies. In some embodiments, more than one gene or its expression product is modulated.

In another embodiment, a method is provided of preventing or treating schizophrenia, or the cognitive deficits associated with schizophrenia by administering midkine to an individual in need. As described herein, midkine has positive effects on symptoms of schizophrenia, and its administration can be a beneficial treatment for the disorder, or the symptoms of the disorder.

Another embodiment of the invention provides a method for screening compounds for their ability to increase or decrease the activity of one or more of the gene targets associated with schizophrenia or the symptoms of schizophrenia. By utilizing animal models of schizophrenia as described herein, and assessing dysregulation of genes associated with schizophrenia, it can be determined which compounds have an influence on this dysregulation. Gene expression can be assessed prior to and after compound administration in a model of schizophrenia, along with assessment of behavioral and biological manifestations. Changes in gene expression or gene expression products can be meaningful with respect to those compounds as potential therapeutics for schizophrenia or the symptoms of schizophrenia.

In another embodiment of the invention, pharmaceutical compositions are provided. The pharmaceutical compositions may comprise one or more of the nucleic acids obtained from the methods of the invention, or as described by the invention. The pharmaceutical compositions may also comprise agonists and antagonists of one or more of the genes described in the invention, in order to treat schizophrenia or the symptoms of schizophrenia.

These and other aspects of the invention may be more clearly understood by reference to the following detailed description of the invention and the appended claims.

### BRIEF DESCRIPTION OF THE FIGURES

FIGS. 1A-1B: Influence of isolation rearing and maternal deprivation on sensorimotor gating in male Wistar rats. Isolation reared animals (n=7-8/group) were raised in single perspex cages from weaning on postnatal day (P) 25. Maternally deprived animals (n=7-8/group) were isolated from their mothers for a single 24-hour period on P9. At the end of the 24-hour period the dams were returned to their litters and left undisturbed until weaning on P25. After weaning on P25 pups were housed in groups of 3-4 per cage. For both models, separate cohorts were analyzed at P30, P40, P60 and P80. Panel A: All isolation reared animals are compared to age-matched social controls (raised in cages of 3-4 from weaning, ns=8/group). Isolated animals exhibited impaired sensorimotor gating (as measured by prepulse inhibition), when analyzed at P60 (F[1.56]=15.38; p=0.0002). This deficit was maintained at P80 (F[1.52]=17.35; p=0.0001). Closed circles represent isolation-reared rats; open circles represent social controls. Panel B: All maternally deprived animals are compared to age-matched controls not maternally deprived (raised in cages of 3-4 from weaning, ns=8/group). Maternally deprived animals did not exhibiting a significant difference in prepulse inhibition compared to controls. Data were analyzed using the two-way ANOVA statistical test. Closed circles represent maternally deprived rats; open circles represent non-deprived controls. Db=decibels.

FIGS. 2A-2B: Influence of isolation rearing and maternal deprivation on spatial learning in male Wistar rats. Isolation reared animals (n=7-8/group) were raised in single perspex cages from weaning on postnatal day (P) 25. Maternally deprived animals (n=7-8/group) were isolated from their mothers for a single 24-hour period on P9. At the end of the 24-hour period the dams were returned to their litters and left undisturbed until weaning on P25. After weaning on P25 pups were housed in groups of 3-4 per cage. For both models, separate cohorts were analyzed at P30, P40, P60 and P80 in a water maze. Panel A: All isolation reared animals are compared to age-matched social controls (raised in cages of 3-4 from weaning, n=8/group). Isolated animals exhibited impaired spatial learning when analyzed at P60 (F[1.19]=36.80; p<0.0001). Closed circles represent isolation-reared rats; open circles represent social controls. Panel B: All maternally deprived animals are compared to age-matched controls not maternally deprived (raised in cages of 3-4 from weaning, ns=8/group). Maternally deprived animals exhibited impaired spatial learning when analyzed at P60 (F[1.19]=5.29; p=0.0222). Data were analyzed using the two-way ANOVA statistical test. Closed circles represent maternally deprived rats; open circles represent non-deprived controls.

FIGS. 3A-3C: Expression of unknown genes in the prefrontal cortex demonstrated by the microarray studies to co-dysregulate with known genes in the medial prefrontal cortex of isolation-reared animals. The known genes are of interest as they are already implicated in schizophrenia-related biology. The unknown ESTs were selected on the basis of their common pattern and, in particular, their substantial up-regulation at P60. Values are represented relative to social controls.

FIGS. 4A-4C: Genes that relate to reelin-Lis1-DISC1 signaling show altered expression in the medial prefrontal cortex of isolation reared animals. Panel A: The inter-relationship between reelin-, Lis1- and DISC1-associated genes. Genes in the shaded circles were dysregulated in the isolation reared animals. Panel B: The altered expression of Lis1 at mRNA level as indicated by the microarray. Panel C: confirmation of change in Lis1 expression by quantitative real time PCR. Open circles represent socially reared controls and closed circles and filled bars represent isolation-reared animals.

FIGS. 5A-5F: Genes that relate to GABAergic transmission and synaptic structure show altered expression in the medial prefrontal cortex of isolation-reared animals. Panels A, C and E: The altered expression of GABAA receptor alpha4, complexin I and synapsin II, respectively, at mRNA level as indicated by the microarray. Panels B, D and F: Confirmation of change in GABAA receptor alpha4, complexin I and synapsin II, respectively, expression by quantitative real time PCR. Open circles and clear bars represent socially-reared controls and closed circles and filled bars represent isolation reared animals.

FIG. 6: Interferon-related gene dysregulation in the medial prefrontal cortex of socially isolated, maternally deprived, and social control animals. Panel A: Interferon-induced protein with tetratricopeptide repeats 2; Panel B: Interferon regulatory factor 7; Panel C: PKR.

FIG. 7: Expression of unknown genes in the prefrontal cortex demonstrated by the microarray studies to co-dysregulate with known genes in the medial prefrontal cortex of maternal deprived animals. The known genes are of interest as they are associated with interferon signaling and, thus, viral infection. Maternal viral infection is associated with increased risk of schizophrenia in the unborn child. The unknown ESTs were selected on the basis of their common pattern and, in particular, their substantial up-regulation at P30. Values are represented relative to social controls.

FIGS. 8A-8B: Influence of prior environmental manipulation on basal dopamine, glutamate and GABA levels in the medial prefrontal cortex of mature Wistar rats on postnatal day 80. Panel A: The concentration of neurotransmitter in dialysate samples collected over a 300 min period is illustrated. Values for social animals (SC; unfilled columns) are compared to those reared in isolation (IR; shaded columns) or with prior maternal deprivation (MD; filled columns) and expressed as the mean and SEM. Those values significantly different (p<0.05) from the control group are indicated with an asterisk. Panel B: Shows the track left by the microdialysis probe with the position of the dialysis membrane indicated with arrowheads.

FIGS. 9A-9C: Consequence of prior environmental manipulation on parvalbumen cell density in the medial prefrontal cortex. Values for social animals (SC; unfilled columns) and those reared in isolation (IR; shaded columns) or with prior maternal deprivation (MD; filled columns) are shown as the mean and SEM and values significantly different from social controls (p<0.05) are indicated with asterisks (* one-tailed t-test; ** two-tailed t-test).

FIGS. 10A-10B: Consequence of prior environmental manipulation on synapse density in the medial prefrontal cortex in animals on postnatal day 80. Values for social animals (SC; unfilled columns) and those reared in isolation (IR; shaded columns) or with prior maternal deprivation (MD; filled columns) are shown as the mean and SEM and values significantly different from social controls (p<0.05) are indicated with asterisks (* one-tailed t-test; ** two-tailed t-test).

FIG. 11: Midkine can reverse isolation rearing-induced sensory processing deficits. Isolation reared animals exhibit a significant deficit in prepulse inhibition (PPI) of startle. Four daily ICV injections of Midkine (2 μg/day; filled circles) reversed PPI behavior back to normal compared to vehicle-treated animals (open circles) (Two-way ANOVA, p<0.05 for treatment).

### DETAILED DESCRIPTION OF THE INVENTION

This invention relates to nucleic acid molecules and their products which have been discovered to be associated with schizophrenia and related disorders and the symptoms thereof via screening methods of the invention, which are described herein. Accordingly, this invention also relates to methods of diagnosing schizophrenia as well as to methods of predicting the susceptibility of an individual to developing schizophrenia and related disorders. This invention also relates to methods of identifying compounds that modulate schizophrenia or the symptoms thereof via manipulations of the nucleic acid molecules and their products. Accordingly, the invention also relates to methods of modulating symptoms of schizophrenia and to treating schizophrenia and related disorders or the symptoms thereof. The invention also relates to methods of treating schizophrenia or the symptoms thereof with midkine

The term “schizophrenia” as used herein encompasses many different mental disorders characterized by psychosis as a core or fundamental feature, including, but not limited to those that are outlined in the Diagnostic and Statistical Manual of Mental Disorders, Fourth Edition (DSM-IV; or the DSM-IV-Text Revision (TR)), or any other diagnostic sources used by mental health care professionals, as well as the individual features and symptoms of the disorders. As used herein, schizophrenia refers to schizophrenia; schizophreniform disorder; schizoaffective disorder (including the bipolar type and the depressive type); schizotypical disorder; schizoid personality disorder; schizotypical personality disorder; delusional disorder (including the erotomaniac type, the grandiose type, the jealous type, the persecutory type, and somatic type, the mixed type, and the unspecified type); brief psychotic disorder (with or without marked stressors, including with postpartum onset); shared psychotic disorder; psychotic disorder due to a general medical condition (including with delusions or hallucinations); substance-induced psychotic disorder (including with delusions or hallucinations, with onset during intoxication, and with onset during withdrawal); and psychotic disorder not otherwise specified, as defined in the DSM-IV, DSM-IV-TR, or any other diagnostic criteria. Further, as used in the invention, schizophrenia encompasses the different schizophrenia subtypes, and various psychotic disorders, including, without limitation, the paranoid type, the disorganized type, the catatonic type, the undifferentiated type, and the residual type, as well as the symptoms associated with these aspect of the disorders and other psychotic disorders.

All possible symptoms of schizophrenia and related disorders are within the scope of this invention, and are included with the use of the term, schizophrenia. The characteristic symptoms of psychosis and schizophrenia include a range of behavioral, cognitive and emotional dysfunctions that include alterations in perception, inferential thinking, language and communication, behavioral monitoring, affect, fluency and productivity of thought and speech, hedonic capacity, volition and drive, and attention. No single symptom is pathognomonic of the diseases, but rather the diagnosis includes the recognition of a constellation of signs and symptoms that are associated with impaired occupational and/or social functioning.

In a clinical evaluation, schizophrenia is commonly marked by two broad categories: positive symptoms and negative symptoms, which are encompassed by the invention. Positive symptoms of schizophrenia and related disorders reflect an excess or distortion of normal functions and include distortions in thought content (delusions), perception of reality (hallucinations, which can be auditory, visual, olfactory, gustatory, and/or tactile), language and thought process (disorganized speech), and self-monitoring of behavior (grossly disorganized and catatonic behavior.

The negative symptoms of schizophrenia are a class of symptoms of schizophrenia which can be considered to reflect a loss or diminution of normal functions. Negative symptoms of schizophrenia and related disorders include affective flattening (characterized by, for example, an immobile and/or unresponsive facial expression, poor eye contact and reduced body language), alogia (poverty of speech' or brief, laconic and/or empty replies), avolition (characterized by a reduced or absent ability to initiate and carry out goal-directed activities), anhedonia (loss of interest or pleasure), social withdrawal, apathy and other negative symptoms known to those of skill in the art.

The positive and negative symptoms of schizophrenia may be assessed using any methodology known in the art including, but not limited to, the Brief Psychiatric Rating Scale (BPRS), the Positive and Negative Symptom Scale (PANSS), the Rorschach Schizophrenia Index (SCZI), and the Scale for the Assessment of Negative Symptoms (SANS), and the Scale for the Assessment of Positive Symptoms (PANS).

Utilizing animal models of schizophrenia, the inventors have observed particular patterns of gene expression which are diagnostic of the disease, and have observed particular patterns of gene expression in the same animal models which are predictive of developing the disease, using the screening methods of the invention. The inventors have discovered a group of previously unknown nucleic acids that are useful for diagnosing schizophrenia. They have also discovered a group of previously identified genes as newly associated with schizophrenia and the symptoms thereof.

As used herein, measuring gene expression refers to detecting any nucleic acid sequence, gene, gene fragment, gene transcript, expressed sequence tags (ESTs) and the like, and are all encompassed by the invention in all aspects.

In one embodiment of the invention, methods for identifying gene targets associated with schizophrenia or the symptoms thereof are provided. Transcriptional regulation is assessed over time in tissue obtained from live animals that are a model of schizophrenia. In some embodiments, only one group of animals is used. When at least two groups of animals are utilized, one of the groups serves as a control group which has not been manipulated or is not an animal model of schizophrenia. Each group contains at least one animal. Gene expression is assessed at different time points relative to the schizophrenia model utilized, and values are compared to those of control animals to determine which transcripts are associated with schizophrenia. For example, with respect to the social isolation model, gene expression may be measured at time points prior to isolation, throughout the isolation period, and after the isolation period, and at the same developmental time points in control animals. In some instances, gene expression is measured prior to the onset of the schizophrenia model, and at times after the model is initiated, so that animals can serve as their own control group, and gene expression after the model onset is compared with gene expression prior to the model onset. In some embodiments, more than two groups of animals are utilized, such that gene expression can be compared among a control group and different models of schizophrenia. The ultimate comparison, whether between a control group and schizophrenia model group, or within a group, prior to and after the onset of the schizophrenia model, is a change in gene expression. The nature of the changes in gene expression assessed are described further herein. Any change in gene expression at any time point can be informative with respect to a gene target's role in schizophrenia or the symptoms thereof.

In some embodiments, gene expression products, rather than gene expression is measured and applied to the same methods described herein, and can be equally informative with respect to what genes and their products are associated with schizophrenia or the symptoms thereof, and which may serve as useful targets.

Any animal model of schizophrenia known in the art is contemplated for use with the methods of the invention, including pharmacological and non-pharmacological models, and models utilizing neurodevelopmental manipulations, as well as manipulations performed prior to birth and genetic manipulations. In a preferred embodiment, the animal model is the social isolation rearing model, which is a widely used, recognized, and validated schizophrenia model (e.g., Geyer et al., Biol. Psychiatry, 34, 361-372, 1993). Social isolation rearing comprises housing an animal in a cage by itself from the time of weaning, and for the duration of experimental procedures. This model can be applied to any species of animal for experimental purposes, all of which are encompassed by the invention. Social isolation rearing leads to a number of behavioral and neurobiological changes which are consistent with typical schizophrenia-related deficits that are observed in schizophrenic patients. As provided in the Examples herein, the inventors have illustrated the power of this model by demonstrating substantial deficits in pre-pulse inhibition, which is a widely used measure of sensorimotor gating, as well as deficits in spatial memory, and, among other things, a reduction in glutamate and paravalbumen cell density in the prefrontal cortex.

Any animal is contemplated in the methods of the invention, though preferably the animal is a mammal. The mammal can include, but is not limited to rodents, including mice, rats, hamsters, voles, guinea pigs, squirrels, prairie dogs, marmots, and gophers. Also within the scope of the invention are non-human primates and avian species. In some embodiments of the invention, humans that either have schizophrenia or the symptoms thereof, or that do not have schizophrenia or the symptoms thereof, or are at risk for developing schizophrenia or the symptoms thereof can provide tissue samples for assessment of gene expression or gene expression products.

Prepulse inhibition reflects a mechanism that allows an individual to filter incoming sensory information such that irrelevant external stimuli are ignored, and important stimuli are attended to (Van den Buuse et al., Curr. Mol. Med., 3, 459-471, 2003). Schizophrenia patients have fundamental deficits in attention and sensory information processing, and these deficits are reflected in prepulse inhibition deficits observed in these patients. These deficits can be reversed in schizophrenia patients by administering, for example, antipsychotic drugs. Prepulse inhibition is a robust phenomenon observed across many species, including humans and rodents, so that it has become a widely used tool in studies of schizophrenia, and a widely accepted means of validating animal models of schizophrenia (Van den Buuse et al., Curr. Mol. Med., 3, 459-471, 2003).

In another preferred embodiment, the animal model is the maternal deprivation model, which includes separating the animals from their mothers for a distinct period of time prior to weaning (e.g., Ellenbroek et al., Schizophr. Res., 30(3), 251-260, 1998). For example, rat pups are isolated from their mothers for a single 24-hour period on postnatal day 9, after which they are returned to their mothers until weaning. This model is also well accepted as a neurodevelopmental model for schizophrenia, leading to pharmacological and behavioral indicators of schizophrenia. As observed in the social isolation model, prefrontal hyperglutamatergia was observed in maternally deprived animals. In addition, maternally deprived animals exhibited a non-statistically significant decrement in pre-pulse inhibition. Without being bound by theory, it is believed that compensatory mechanisms in the maternal deprivation model may prevent full emergence of schizophrenic symptoms. In this regard, it is interesting to note that in addition to the hypoglutamatergia, maternally deprived animals also exhibit a concomitant prefrontal hypergabergia which could represent a component of this proposed protection adaptation facilitating normal sensorimotor function. Importantly, treatment of the maternally deprived animals with the anti-psychotic clozapine actually unmasks a PPI deficient and normalises the GABA levels in the prefrontal cortex further supporting the hypothesis that high GABA levels in the maternal deprivation animals may indeed represent some form of compensation mechanism that restores normal mPFC function even in the presence of low glutamate.

Pharmacological models of schizophrenia are also contemplated by the invention, including those which are based on alterations in the dopamine, GABA, glutamate, and serotonin systems (Van den Buuse et al., Curr. Mol. Med., 3, 459-471, 2003). Also contemplated by the invention are models which involve neonatal brain lesions of the hippocampus, the amygdyla, the frontal cortex, and any other brain region with a potential role in schizophrenia or the symptoms thereof, as well as prenatal and neonatal infection models and other prenatal and neonatal models such as early exposure to anesthetics, cannabinoids, epidermal growth factor, and ethanol (Van den Buuse et al., Curr. Mol. Med., 3, 459-471, 2003).

One example of a pharmacological model encompassed by the invention is the phencyclidine (PCP) model of schizophrenia, which suggests that N-methyl—aspartate (NMDA) receptor hypofunction and its consequences may play an important role in the pathophysiology of schizophrenia (e.g., Jentsch and Roth, Neuropsychopharmacology, 20(3), 201-225, 1999). The schizophreniform psychosis caused by PCP resembles schizophrenia in all of the relevant domains of psychopathology, especially with respect to the negative symptoms and cognitive dysfunction. In this model, animals are administered PCP or an analogue via systemic injection, and within thirty minutes, symptoms are apparent. This model is often utilized in rodents and non-human primates, but humans that abuse the drug may also be considered within the scope of the invention. Another pharmacological model used is ketamine administration, which antagonizes NMDA receptors and its administration is thought to resemble the psychotic state, and in particular, the delusions associated with schizophrenia, and to provide a window to the early stages of the disease process (e.g., Lahti et al., Neurophychopharmacology, 13, 9-19, 1995).

In some embodiments of the invention, transcriptional regulation is assessed from brain tissue. Many brain regions are implicated in schizophrenia and the symptoms thereof, which are all contemplated as useful in the methods of the invention (e.g., Pinkham et al., Schizophr. Res., 2007). These brain regions include, but are not limited to the prefrontal cortex, anterior cingulate gyms, hippocampus, the cortex, neocortex, amygdala, striatum, caudate nucleus, temporal lobes, corpus callosum, and cerebellum. These regions, among others, can all be used to assess for transcriptional regulation prior to, during, and after the manipulation which initiates the schizophrenia model. In a preferred embodiment, the brain tissue assessed is from the medial prefrontal cortex. It is also contemplated by the invention that transcriptional regulation can be assessed from other tissue including without limitation, blood, plasma, lymph, rine, mucus, sputum, saliva, CSF, or tissue from other organs of the body.

Depending on the schizophrenia model utilized with the methods of the invention, transcriptional regulation is assessed from tissue at various time points relative to the induction of the model. In embodiments using the social isolation model, for example, transcriptional regulation may be assessed at one or more time intervals prior to (for baseline comparisons) and following isolation (e.g., postnatal day 25) in experimental animals, and at the same or different time intervals for socially-reared control animals. Gene expression is measured in all groups of animals, for ultimate comparison to the gene expression of control animals. Gene expression that varies in the schizophrenia model as compared to the control animals is indicative of potential relevance of the transcript or its products' role in schizophrenia. Changes in gene expression observed prior to symptom onset in that particular model can be informative with respect to prodromal gene markers, and changes in gene expression concurrent with or following the presence of symptoms in the model used can be informative with respect to diagnostic markers of the disease.

In some embodiments of the invention, expression of genes that have previously been identified as associated with schizophrenia or the symptoms of schizophrenia are particularly useful, as changes in their regulation relative to control animals can be informative with respect to identifying novel transcripts or other genes that are newly identified as associated with schizophrenia. Changes in known genes previously identified as associated with schizophrenia at particular time points can be used to match with unknown transcripts, or with genes not previously associated with schizophrenia, thereby indicating a role for those transcripts or genes in schizophrenia.

Therefore, the expression of the new transcripts (or newly associated genes or transcripts) that are identified as regulated following the onset of the schizophrenia model are compared with the expression genes previously identified as being associated with schizophrenia or the symptoms thereof at the same time points. The previously identified genes can be chosen from any gene or transcript that has been previously associated with schizophrenia. For example, they can include, but are not limited to, complexin 1, GABAARα4, synapsin 2, parvalbumin, Lis-1, DISC1, DISC2, DIS1 reelin, neuregulin-1, COMT, dysbindin, G72, G30, DTNBP1, DAO, DAOA, brain-derived neurotrophic factor, Akt, DAAO, GRIN2B, RGS4, GRM3, calcineurin, α-7 nicotinic receptor gene, PRODH2, CAPON, TRAR, PPP3CC, midkine, transthyretin, USAG-1, and eNNP2.

In one embodiment, the average temporal pattern of the genes previously identified as associated with schizophrenia is used as a “seed pattern” and all regulated genes are ranked for their similarity to this pattern based on the time points and amplitude as described herein. This seed pattern may include 2 or more previously identified genes. In some embodiments of the invention, the seed pattern may include 5 or 10 identified genes, and in some embodiments in may include 20, 30, 50, 100, 200, 500, 1000 or more previously identified genes. In one embodiment, the top 100 matches to the seed pattern are chosen as the transcripts with the strongest relevance for schizophrenia. In another embodiment, the top 50 matches to the seed pattern are chosen as the transcripts with the strongest relevance for schizophrenia. In yet another embodiment, the top 25 matches to the seed pattern are chosen as the transcripts with the strongest relevance for schizophrenia.

Any detectable change in amplitude of gene expression is included as meaningful for identifying transcripts associated with schizophrenia or the symptoms thereof. The change in amplitude of gene expression may be an upregulation or downregulation. Any degree of change in amplitude may be relevant and within the scope of this invention provided such change is sufficiently correlated with development of or expression of schizophrenic symptoms. Such changes in amplitude may, without limitation be a change of 1%, 10%, 50%, or 100%, and in some embodiments the change in amplitude of gene expression may be 3-fold, 5-fold, 10-fold, 20-fold, 50-fold, 100-fold or greater. In some embodiments, no change in gene expression at one or more of the assessed time points may be meaningful, particularly in the context of gene expression at nearby time points, and is within the scope of this invention. The change in expression can be either an increase or a decrease from baseline expression, or it may be an increase or decrease in gene expression relative to the gene expression at other time points within the temporal pattern, or it can be an increase or decrease relative to control animals at the same time point. The gene transcripts identified according to the methods of the invention are indicative of their role in schizophrenia. Compounds that increase or decrease the activity of the genes, gene fragments, and gene products identified as targets associated with schizophrenia using the methods of the invention described above, may also be drug candidates for modulating schizophrenia or its symptoms, or for preventing or delaying the onset of schizophrenia.

Changes in gene expression over any period of time relative to the onset of schizophrenia model used are within the scope of this invention and may vary greatly depending on the species that is evaluated and the model that is used. In some embodiments, depending on the model utilized, the gene expression is regulated within 6 months of the onset of the model. In another embodiment, the gene expression is regulated within one week of the onset of the model. In another embodiment, the gene expression is regulated within one month of the onset of the model. In yet another embodiment, the gene expression is regulated within two months of onset of the model, and in some embodiments, the gene expression is regulated within three months of the onset of the schizophrenia model. In another embodiment, the gene expression is regulated 10 minutes, 20 minutes, 30 minutes, one hour, two hours, four hours, six hours, or twelve hours after the onset of the model. In some embodiments the gene expression is regulated within one year of the onset of the model, within five years of the onset of the model, or within ten or more years of the onset of the model.

For example, when utilizing the social isolation rearing model of schizophrenia, measurement of gene expression or the products thereof can be performed prior to isolation and at postnatal days 30, 40, 60, and 80. When utilizing the maternal deprivation model of schizophrenia, measurement of gene expression can be performed, for example prior to deprivation and at postnatal days 30, 40, 60, and 80. As another example, when the schizophrenia model is PCP, gene expression may be measured prior to drug administration, and 10, 20, 30, 60, and 120 minutes after drug administration. In humans, in the case of a drug-induced psychosis, for example, gene expression or the products thereof may be measured prior to drug administration and at 10, 20, 30, 60, and 120 minutes after drug administration, as well as up to several days and months following drug administration. As another example, in humans, after an early life stressor which may lead to schizophrenia or the symptoms thereof, gene expression or the products thereof may be measured within hours of the event, prior to the event, and after 1, 2, 3, 4, 5, 6, 12, and 24 months after the event, as well as up to 10, 20, and 30 years after the stressful event.

In some embodiments of the invention, gene expression products are assessed relative to the schizophrenia model or models utilized, and amplitude and time points comparisons are carried out as described herein for gene expression. According to the invention, gene expression products include any products which have been or may be determined to be associated with, or be capable of modulating schizophrenia or the symptoms thereof, but are not limited to proteins, peptides, or nucleic acid molecules (e.g., mRNA, tRNA, rRNA, or cRNA) that are involved in transcription or translation.

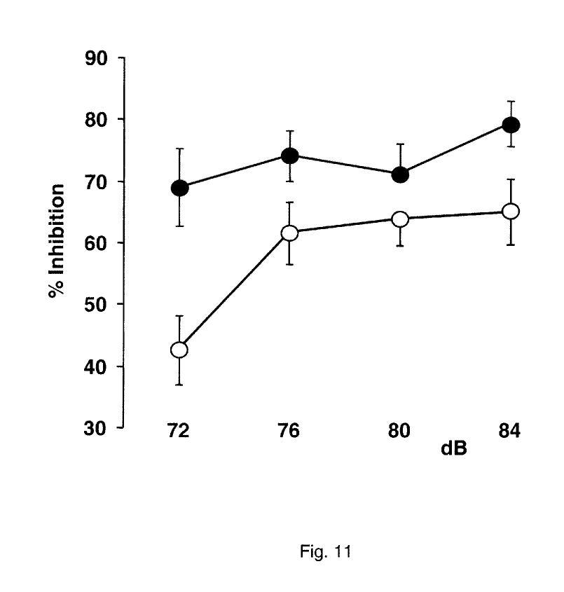

In one embodiment, the social isolation rearing model is used in rats, and gene expression is measured from the prefrontal cortex in rats that undergo isolation, as well as in control, socially reared animals at postnatal days 30, 40, 60, and 80. In some embodiments, mRNA of at least one or more of complexin 1, GABAARα, synapsin 2, parvalbumin, and the genes from FIGS. 11 through 14 are measured at the time points indicated. In other embodiments, mRNA of at least one or more of interferon-induced protein, interferon regulatory factor 7, PKR, Ifi44, Ifit2, Irf7, Isgf3g, Glp2. Mx1, Mx2, and SEQ ID NO: 1 through SEQ ID NO: 16 are measured at the time points indicated.

According to the methods of the invention, gene expression levels may be detected by methods known to those skilled in the art and may be obtained, for example using any apparatus that can measure gene expression levels which are widely known in the art. The nucleic acid molecule levels measured can be derived directly from the gene or, alternatively, from a corresponding regulatory gene. All forms of gene expression products can be measured, including, for example, spliced variants. Similarly, gene expression can be measured by assessing the level of protein or derivative thereof translated from mRNA. This may, however, also reflect posttranslational modifications and other forms of processing. If the gene expression assessed is at the mRNA level, it can, for example, also be measured by in situ hybridization, Northern blot analysis, dot-blot hybridization analysis, microarray analysis, or by PCR. Such methods are described in detail, for example, in Ausubel et al., Current Protocols In Molecular Biology (New York: John Wiley & Sons) (1998); and Sambrook et al., Molecular Cloning: A Laboratory Manual, 2nd edition (New York: Cold Spring Harbor University Press (1989).

In another embodiment of the invention, gene expression levels can be obtained by contacting the sample of interest with a suitable microarray, and determining the extent of hybridization of the nucleic acid in the sample to the probes on the microarray. It is also contemplated by the invention that gene expression assessed by methods such as microarray can be validated by using quantitative real-time PCR analysis at the time points determined to be relevant from the temporal profile of gene expression observed. It will be apparent to those skilled in the art that any methodology that can be utilized for measuring gene expression will be suitable for use in the invention. Microarray analysis and PCR analysis can be carried out according to the Examples described herein, or by any of the available methods known in the art (see, e.g., Avison, Measuring Gene Expression, Taylor & Francis Group, NY, N.Y., 2007).

If the gene expression product is a protein or polypeptide, transcriptional regulation can be measured using techniques for protein detection and quantification that are known in the art. Antibodies, for example, can be generated which are specific to the protein using routine methods known in the art, which specifically bind to the protein of interest so that they can be detected and measured. Protein measurement can be carried out by any methods known in the art, including histochemistry, immunoblot analysis, in vitro binding studies, radioimmunoassay, and ELISAs.

In one embodiment, the transcripts which are identified as associated with schizophrenia or the symptoms thereof are expressed sequence tags (ESTs). ESTs are short single-pass sequence reads from mRNA (cDNA). They may be of various lengths. For use in this invention, it is preferred, but not required, that they be of sufficient length to identify a unique expressed sequence. Typically, they are about 300-500 bp in length. However, sequences as short as about 16 bases may be sufficient to identify a specific sequence. ESTs represent a snapshot of genes expressed in a given tissue and/or at a given developmental stage. They are tags (some coding, others not) of expression for a given cDNA library. There are now well over one million of these sequences in the publicly available database and these sequences are believed to represent more than half of all human genes. The ESTs of, and for use with the invention, however, are not meant to be limited by what is available in public databases, and also may be novel ESTs that are generated and identified according to the methods of the invention described herein.

The ESTs detected according to the invention are useful for elucidating the genes and gene products responsible for regulating schizophrenia, the symptoms thereof, and the onset of schizophrenia and therefore for understanding the mechanisms underlying schizophrenia and the symptoms thereof. The ESTs detected according to the methods of this invention demonstrate specific regulation at time points relative to the schizophrenia models utilized. Such involvement may be to contribute, or be required for the onset of and maintenance of schizophrenia or the symptoms thereof. For the purposes of the invention, it is not necessary to know what genes the ESTs are associated with, because changes in amplitude at particular time points relative to the schizophrenia model or models utilized are useful in themselves as markers for diagnosing and predicting the onset of schizophrenia or the symptoms thereof.

Once these ESTs are identified as being associated with schizophrenia or the symptoms thereof, they are useful for detecting or diagnosing schizophrenia either prior, to or concurrent with the appearance of its symptoms, as well as for modulating the onset of schizophrenia or the symptoms thereof by either enhancing or inhibiting EST expression or the products thereof.

Similarly, the ESTs for use with the invention are useful for development of new pharmaceutical agents for treatment of or prevention of schizophrenia or the symptoms thereof. Pharmaceutical agents may be useful to modulate relevant ESTs identified as associated with schizophrenia or the symptoms thereof, to either increase or decrease their expression or the products thereof.

Systems for carrying out all of the methods described herein are also provided, and optionally comprise a computer system and related software. For example, as relates to the foregoing method, the system may comprise groups of mice in which an animal model of schizophrenia in living animals has been initiated, and a computer system comprising software, said computer system configured to assess transcriptional regulation in tissue over time in animals that are a model of schizophrenia, wherein the tissue is sampled one or more times after the initiation of the model and optionally one or more times prior to the initiation of the model; compare the transcriptional regulation from prior to initiation of the model with transcriptional regulation from after the initiation of the model, and/or with transcriptional regulation assessed from tissue in living animals not subject to a schizophrenia model; and detect a transcript that is dysregulated in tissue from animals that are a model of schizophrenia. Optionally, the computer system may output a result which is indicative of gene targets associated with schizophrenia or schizophrenia symptoms.

In one embodiment, the invention comprises nucleic acid sequences obtained according to the screening methods of the invention. In another embodiment, the invention comprises a nucleic acid sequence selected from the group consisting of SEQ ID NO:1 through SEQ ID NO:16.

In yet another embodiment of the invention, a method is provided of predicting the susceptibility to schizophrenia or the symptoms thereof in an individual. A biological sample is provided by an individual to determine the susceptibility to onset of schizophrenia or the symptoms thereof, and from the sample provided gene expression or gene expression products are measured which have been identified to be informative regarding the onset of schizophrenia or the symptoms thereof; these genes are considered “pre-symptomatic genes” and are described herein. If the gene expression or products thereof measured are considered to be dysregulated, then there is an increased likelihood for developing schizophrenia or the symptoms thereof, compared to an individual who does not demonstrate dysregulation of those same genes or products thereof or compared to an earlier time point within the same individual, in which dysregulation of those same genes or products thereof was not demonstrated.

Dysregulation refers to any change in gene expression or the products thereof relative to the gene expression or products thereof in an individual, at the same general time point or time points, who have been determined to not have ever presented with symptoms of schizophrenia or who have never developed schizophrenia. It can also refer to any change in gene expression or the products thereof relative to a baseline measure in the same individual, at a time, for example, when certain external or internal factors that initiate the onset of schizophrenia were not present or had not yet caused pathologic or prepathologic changes.

The inventors have identified several genes, the dysregulation of which are predictive of the onset of schizophrenia, or the susceptibility to schizophrenia in a widely accepted and validated animal model of schizophrenia. These genes have been discovered to be associated with schizophrenia or the symptoms thereof. Utilizing the screening methods described herein, the inventors have identified genes that are dysregulated prior to the onset of the symptoms of schizophrenia, including interferon-induced protein, interferon regulatory factor 7, PKR, Ifi44, Ifit2, Irf7, Isgf3g, Glp2. Mx1, Mx2, SEQ ID NO: 11 through SEQ ID NO: 16, and in some embodiments, the genes or fragments listed in Tables 3, 4, 7, and 8.

As demonstrated in the Examples described herein, when utilizing the social isolation rearing model for schizophrenia in rats, symptoms are present on postnatal day 60, including deficits in prepulse inhibition and the neurobiological measures described herein. In assessing gene expression at various intervals from animals after initiating the schizophrenia model, it was determined that certain genes exhibited dysregulation (relative to control animals) prior to the time of symptoms on postnatal day 60, including genes exhibiting dysregulation (relative to control animals) at postnatal day 30 and postnatal day 40. Accordingly any transcripts or genes identified as dysregulated at any time point prior to the onset of symptoms in a model of schizophrenia can be used as predictive of schizophrenia or the symptoms thereof, or as a drug target and are within the scope of this invention. Also within the scope of the invention is the detection of gene expression or product dysregulation that occurs at a point where just some symptoms of schizophrenia are present, but prior to a set of circumstances that are diagnostic of schizophrenia.

As described above, the genes or the products thereof for use with the invention include, but are not limited to proteins, peptides, or nucleic acid molecules (e.g., mRNA, tRNA, rRNA, or cRNA) that are involved in transcription or translation. Also as described above, any detectable change in gene expression, or the products thereof, whether upregulated or downregulated, are contemplated as useful in the methods of the invention, as well as any time points prior to the onset of schizophrenia or the symptoms thereof. Any methods available and known to one of ordinary skill in the art are contemplated for measuring gene expression or the products thereof, as described herein.

In some preferred embodiments of the invention, the relevant genes for which expression or expression products are identified as being dysregulated include, but are not limited to interferon-induced protein, interferon regulatory factor 7, PKR, Ifi44, Ifit2, Irf7, Isgf3g, Glp2. Mx1, Mx2, and SEQ ID NO: 11 through SEQ ID NO: 16. In yet other embodiments of the invention, the relevant genes whose expression or expression products are identified as being dysregulated include those described in Tables 3, 4, 7, and 8. In another embodiment of the invention, the relevant genes for which expression or expression products are identified as being dysregulated have been previously identified as being associated with schizophrenia or the symptoms thereof. In another embodiment of the invention, one or more genes identified as being dysregulated include, but are not limited to genes that are involved in synaptogenesis, synaptic pruning, synaptic drive, synaptic communication, synapse formation, synaptic activity, synaptic plasticity, neuriotogeneis, neurite architecture, neuronal migration, intracellular transport, integrator genes, signal transduction, microtubule assembly, axon elongation, cell motility, and G-protein coupled receptor signaling. All of these genes, collectively, which may be used to predict the onset or susceptibility to schizophrenia or the symptoms thereof, are referred to herein as “pre-symptomatic” genes.

In one embodiment of the invention, in determining the susceptibility to schizophrenia or the symptoms thereof, one or more of the pre-symptomatic genes are downregulated in an individual prior to the onset of schizophrenia or the symptoms thereof, which is informative of the susceptibility of the individual to schizophrenia or the symptoms thereof. In another embodiment, one or more of the pre-symptomatic genes are upregulated in an individual prior to the onset of schizophrenia or the symptoms thereof, which is informative of the susceptibility of the individual to schizophrenia or the symptoms thereof. In some embodiments, one or more of the pre-symptomatic genes will be upregulated, and one or more of the symptomatic genes will be downregulated, which is informative of the susceptibility of the individual to schizophrenia or the symptoms thereof.

In some embodiments, one gene or its expression product is predictive of the susceptibility to schizophrenia, and in other embodiments, two or more genes or their expression products are predictive of the susceptibility to schizophrenia or the symptoms thereof.

In some embodiments, the individual providing the biological sample is an individual that possess at least one or more risk factors for schizophrenia. Risk factors contemplated by the invention include, but are not limited to family history of schizophrenia, schizophrenia-like psychoses, and other mental disorders; genetic factors; place of birth, including urbanicity; season of birth; obstetric complications; infections; diet; toxic exposures; household crowding; exposure to pets; breast-feeding; family history of Gaucher's disease; maternal infection, such as herpes simplex virus-2; prenatal and/or postnatal stress; enhanced maternal immune activation; handedness; childhood exposure to social adversity, including social defeat or social exclusion; and alterations in neurotransmitter levels, including, but not limited to dopamine, glutamate, GABA, acetylcholine, and serotonin. Any number of these factors either alone or in combination can be indicative of increased risk of developing schizophrenia or the symptoms thereof, which may be an indication for assessing the genes identified by the inventors and identified by the methods described herein, to determine the susceptibility to developing schizophrenia or the symptoms thereof. The presence of one or more risk factors may also be useful in determining the susceptibility of an individual to schizophrenia, when used in combination with detection of pre-symptomatic gene dysregulation. In some embodiments, the individual does not have any known risk factors for schizophrenia.

In some embodiments of the invention, the individual is completely asymptomatic, presenting with no known symptoms of schizophrenia. In other embodiments, the individual presents with one or more symptoms of schizophrenia, which include, but are not limited to the symptoms described herein, such as any of the positive and negative symptoms characterized by clinicians. Any potential symptom that is indicative of schizophrenia is within the scope of the invention. The individual may present with one or more symptoms that, taken together, do not constitute a clinical diagnosis for schizophrenia or related disorders, as for example, defined in the DSM-IV. For example, the individual may not present with a sufficient number of symptoms, they may not have had the symptoms for a sufficient duration, or they may present with symptoms which resemble schizophrenia or a related disorder, but for some reason do not fit into the defined categories for clinical diagnosis and therefore would not be diagnosed by a clinician as having schizophrenia or a related disorder.

The biological sample from which gene expression or a product thereof is measured can include any biological tissue provided by an individual. In particular, genes or the expression products thereof can be evaluated from blood, plasma, saliva, CSF, urine, lymph, sputum, or other tissues of the body. In a preferred embodiment, the measurement is made from peripheral blood mononuclear cells.

In another embodiment, this invention provides methods of diagnosing schizophrenia or the symptoms thereof in an individual. A biological sample is provided by an individual, to determine if the individual has schizophrenia or the symptoms thereof, and from the sample provided, gene expression or the products thereof are measured which have been identified to be informative regarding diagnosis of schizophrenia; these genes are considered “symptomatic genes” and are described herein. If the gene expression or products thereof are considered to be dysregulated, it is diagnostic of schizophrenia. The presence of dysregulation or more than one symptomatic gene is greater evidence of the presence of schizophrenia.

Dysregulation refers to any change in gene expression or the products thereof relative to the gene expression or products thereof in an individual, at the same general time point or time points, who have been determined not to have schizophrenia, or in some embodiments, who have never developed schizophrenia, or who never develop schizophrenia. It can also refer to any change in gene expression or the products thereof relative to a baseline measure in the same individual, at a time, for example, prior to the onset of schizophrenia, and in some embodiments, prior to the onset of any symptoms of schizophrenia.

The inventors have identified several genes, the dysregulation of which are indicative of the presence of schizophrenia or the symptoms thereof in a widely accepted and validated animal model of schizophrenia, and which can be considered as biomarkers for schizophrenia and the symptoms thereof. Using the methods of the invention as described herein, the inventors have discovered many genes to be associated with schizophrenia or the symptoms thereof, including but not limited to those listed in Tables 5, 6, 9, and 10, and SEQ ID NO: 1 through SEQ ID NO: 10. In another embodiment of the invention, one or more genes identified as being dysregulated include, but are not limited to genes that are involved in synaptogenesis, synaptic pruning, synaptic drive, synaptic communication, synapse formation, synaptic activity, synaptic plasticity, neuriotogeneis, neurite architecture, neuronal migration, intracellular transport, integrator genes, signal transduction, microtubule assembly, axon elongation, cell motility, and G-protein coupled receptor signaling. Many genes that have been previously associated with schizophrenia are within the scope of the invention, including GABAA receptor α4, complexin 1, and synapsin 2, parvalbumin, and Lis-1. Further, the inventors have identified several novel ESTs, the dysregulation of which can serve as biomarkers for schizophrenia or the symptoms thereof. Utilizing the screening methods described herein, comprising assessing gene transcription or the products thereof over time in animal models of schizophrenia, the inventors have identified genes that are dysregulated concurrent with or after the onset of the symptoms of schizophrenia. As demonstrated in the Examples described herein, when utilizing the social isolation rearing model for schizophrenia, symptoms are present on postnatal day 60, including deficits in prepulse inhibition and the neurobiological measures described herein. In assessing gene expression at various intervals from animals after the onset of the schizophrenia model, it was determined that certain genes exhibited dysregulation (relative to control animals) concurrent with the presence of symptoms on postnatal day 60. In some instances, dysregulation was also observed on postnatal day 80. Within the scope of the invention for use in diagnosis are any transcripts or genes identified as dysregulated at any time point concurrent with, and after the onset of symptoms in a model of schizophrenia.

As described in other embodiments herein, detecting dysregulation of relevant genes, transcripts, or their expression products, as used herein, refers to any difference in gene expression or gene expression product relative to individuals who have been assessed at the same general time point, concurrent with or after the onset of schizophrenia or the symptoms thereof.

As described above, the genes or the products thereof for use with the invention include, but are not limited to proteins, peptides, or nucleic acid molecules (e.g., mRNA, tRNA, rRNA, or cRNA) that are involved in transcription or translation. Also as described above, any detectable change in gene expression, or the products thereof, whether upregulated or downregulated, are contemplated as useful in the methods of the invention, as well as any time points concurrent with or following the onset of schizophrenia or the symptoms thereof. Any methods available and known to one of ordinary skill in the art are contemplated for measuring gene expression or the products thereof, as described herein.

In some embodiments of the invention, the relevant genes whose expression or expression products are identified as being dysregulated concurrent with, or following the onset of schizophrenia or the symptoms thereof include genes or transcripts, or the expression products thereof, that have not previously identified as associated with schizophrenia or the symptoms thereof. These include, but are not limited to the ESTs identified according to the invention, including SEQ ID NO:1 through SEQ ID NO:10. Also contemplated for use in the invention are the genes identified in Tables 3 through 10, which the inventors have discovered to be associated with schizophrenia or the symptoms thereof using the methods described herein. Also within the scope of the invention for purposes of diagnosis or predicting onset of schizophrenia or the symptoms thereof are genes which have been previously identified as associated with schizophrenia or the symptoms thereof, including any of the genes identified herein as associated with schizophrenia or the symptoms thereof, and any genes identified according to the methods of the invention. In another embodiment of the invention, one or more genes identified as being dysregulated include, but are not limited to genes that are involved in synaptogenesis, synaptic pruning, synaptic drive, synaptic communication, synapse formation, synaptic activity, synaptic plasticity, neuriotogeneis, neurite architecture, neuronal migration, intracellular transport, integrator genes, signal transduction, microtubule assembly, axon elongation, cell motility, and G-protein coupled receptor signaling. All of these genes and transcripts, collectively, are referred to as “symptomatic” genes.

In one embodiment of the invention, one or more of the symptomatic genes is downregulated in an individual concurrent with or following the onset of schizophrenia or the symptoms thereof, and is informative as a diagnostic marker. In another embodiment, one or more of the symptomatic genes is upregulated in an individual concurrent with or following the onset of schizophrenia or the symptoms thereof, and is informative as a diagnostic marker. In some embodiments, one or more of the symptomatic genes is upregulated, and one or more of the symptomatic genes is downregulated, which is informative as a diagnostic marker. In some embodiments, a distinctive pattern of gene expression regulation, comprising more than one time point of regulation is informative for diagnosis or prediction of schizophrenia or the symptoms thereof.

In another embodiment of the invention, the symptomatic gene, genes, or the expression products thereof that are dysregulated have been previously identified as being associated with schizophrenia or the symptoms thereof.

In some embodiments, one symptomatic gene or its expression product is a biomarker for schizophrenia or the symptoms thereof, and in other embodiments, two or more symptomatic genes or their expression products are biomarkers for schizophrenia or the symptoms thereof.

It is of particular importance to utilize one or biomarkers as described herein for diagnosing schizophrenia or the symptoms thereof because traditional diagnoses on their own are not always accurate. For example, inaccurate diagnosis can be the result of overlap in diagnostic criteria between mood and psychotic disorders, provider bias, miscommunication between patient and provider, changes in diagnostic criteria, differences in diagnostic practice between providers, assessment at a time when symptom acuity is severe, influence of substance abuse, and a lack of sufficient data obtained. In addition, cultural differences can lead to inaccurate diagnoses; for example, multiple studies have shown that significant disparities exist in the diagnosis of schizophrenia between African Americans and Caucasians (e.g., Hampton, Br. J. Psychiatry Suppl., 50, s46-51, 2007). The use of the symptomatic genes may be able to yield a reliable diagnosis of schizophrenia or the symptoms thereof in advance of traditional diagnostic tools which often rely on the presence of a certain number of symptoms for a specified amount of time. Therefore, with earlier diagnosis, earlier treatment and care of patients can be initiated.

Evaluation of dysregulation of symptomatic genes or their expression products may be performed alone or in combination with one or more of any clinical, behavioral, or biological assessment tools used for diagnosing schizophrenia or the symptoms thereof. Use of the symptomatic genes or their expression products as described herein can be particularly powerful when used in combination with other diagnostic tools. For example, they may be utilized in conjunction with diagnoses made in accordance with the DSM-IV (or DSM-IV-TR); the Comprehensive Assessment of Symptoms and History (CASH), or an adapted version using a cultural formulation to make the instrument more culturally sensitive (CASH-CS); the Positive and Negative Syndrome Scale (PANSS) for typological and dimensional assessment; the Brief Psychiatric Rating Scale (BPRS); the Rorschach Schizophrenia Index (SCZI); the Scale for the Assessment of Negative Symptoms (SANS); the Scale for the Assessment of Positive Symptoms (PANS); the Bonn Scale for the Assessment of Basic Symptoms (BSABS); the Instrument for the Retrospective Assessment of the Onset of Schizophrenia; the Present State Examination; the Structured Interview for Prodromal Syndromes; the Global Assessment of Functioning; neuroimaging; use of other biomarkers or genetic factors; family history of psychotic disorders; and assessment of any risk factor for schizophrenia or the symptoms thereof.

In another embodiment, the targets obtained according to the methods of the invention, as well as the targets described herein, are used to prevent schizophrenia or the symptoms thereof by either upregulating or downregulating them, or by increasing of decreasing their products. Depending on the nature of the dysregulation of the target in association with predicting susceptibility to schizophrenia or the symptoms thereof, it may be desirable to increase or decrease the function of the gene of interest, or a product of the gene of interest.

Included among the gene targets to be modified for preventing schizophrenia or the symptoms thereof are the pre-symptomatic genes as described herein. In some embodiments it will be necessary to modify only one such gene or gene product, and in other embodiments it may be desirable to modify more than one pre-symptomatic gene or gene product. In one embodiment, the genes to be modified include interferon-induced protein, interferon regulatory factor 7, or PKR, or a combination thereof. As an example, it may be desirable to increase the expression of these genes or their products, as they have been found to be decreased under circumstances leading to the onset of schizophrenia as compared to circumstances that do not lead to schizophrenia. In another embodiment, it may be useful to modify one or more of the genes from Tables 3, 4, 7 and 8. In other embodiments, it may be useful to modify one or more of the genes from Tables 5, 6, 9, and 10. In yet other embodiments, it may be useful to modify one or more of SEQ ID NO:1 through SEQ ID NO: 16.

In another embodiment, the targets obtained according to the methods of the invention, as well as the targets described herein, are used to treat schizophrenia or the symptoms thereof by either upregulating or downregulating them, or by increasing of decreasing their products. Depending on the nature of the dysregulation of the target in association with schizophrenia or the symptoms thereof, it may be desirable to increase or decrease the function of the gene of interest, or a product of the gene of interest.

Included among the gene targets to be modified for treating schizophrenia or the symptoms thereof are the symptomatic genes as described herein. In some embodiments it will be necessary to modify only one such gene or gene product, and in other embodiments it may be desirable to modify more than one symptomatic gene or gene product. Included among the gene targets to modulate for treating schizophrenia or the symptoms thereof are nucleic acid sequences selected from the group consisting of SEQ ID NO:1 through SEQ ID NO:16. Any one or more of the genes from Tables 5, 6, 9, and 10 or their products, may also be modulated for treating schizophrenia or the symptoms thereof. As an example, it may be desirable to decrease the expression of these nucleic acids (e.g., the ESTs) or their products, as they have been found to increase in association with schizophrenia relative to circumstances where no symptoms of schizophrenia are present. In other embodiments, it may be useful to modulate one or more of the genes from Tables 3, 4, 7, and 8.

In some embodiments, it may be desirable to modulate the expression or expression product of at least one pre-symptomatic gene in combination with modulating the expression or expression product of at least one symptomatic gene, and in some cases this combination for treatment or prevention of schizophrenia or the symptoms thereof may be enhanced by the addition of other treatments.

In some embodiments, treatment by modulating symptomatic or pre-symptomatic genes or their products might be indicated for individuals for whom other schizophrenia treatments are ineffective or to which they have become resistant, or for individuals who have had improved symptoms but experienced a relapse or no further improvement.

In some embodiments, the individual receiving treatment or prophylactic treatment is an adult, in some embodiments the individual is an adolescent, and in some embodiments the individual is a child.

In some embodiments, prevention of schizophrenia or the symptoms thereof by modulating at least one pre-symptomatic gene or expression product, or treatment of schizophrenia or the symptoms thereof by modulating at least one symptomatic gene or expression product may be performed in combination with one or more other preventative or treating agents or regimens. For example, the prevention or treatment methods described herein can be combined with traditional behavioral or drug therapies. These therapies can be those administered to treat schizophrenia, or to target individual symptoms of schizophrenia. These therapies include, but are not limited to cognitive-behavioral therapy; social skills training; psychotherapy; cognitive remediation; family intervention; first and second generation antipsychotics, such as haloperidol, clozapine, risperidone, and olanzapine; neuroleptics such as chlorpromazine and paliperidone; quetiapine; aripiprazole; ziprasidone; trifluperazine; flupenthixol; loxapine; perphenazine; fluvoxamine; iloperidone; osanetant; MEM 3454; ORG 5222; DU 127090; DTA 201A; psychostimulants; drugs that act as dopamine or acetylcholine antagonists; dopamine reuptake inhibitors; glutamate antagonists; serotonin antagonists; drugs that enhance cognitive abilities; electric shock therapy; eicosapentaenoic acid; hormone therapy, such as testosterone; and antidepressants.

According to the invention, gene expression products include, but are not limited to proteins, peptides, or nucleic acid molecules (e.g., mRNA, tRNA, rRNA, or cRNA) that are involved in transcription or translation.

In one embodiment of the invention, gene expression products can refer to epigenetic changes such as DNA methylation and chromatin remodeling. Chromatin remodeling is initiated by the posttranslational modification of the amino acids that make up histone proteins, or by the addition of methyl groups to the DNA at CpG sites to convert cytosine to 5-methylcytosine. Any epigenetic modulation known to one of skill in the art is contemplated by the invention.

Any means of enhancing gene expression, or the gene expression product in an individual that is known by one of skill in the art may be used to modulate gene expression or a gene expression product as contemplated for methods of prevention or treatment in the invention. For example, pharmaceutical compositions which are agonists for the gene product of interest may be used to prevent or treat schizophrenia or the symptoms thereof.

Any means of reducing gene expression, or the gene expression product in an individual that is known by one of skill in the art may be used to reduce gene expression or a gene expression product as contemplated for methods of prevention or treatment in the invention. Non-limiting examples for use in reducing gene expression or gene expression products according to the invention include RNA interference, antisense RNA, antibodies, and pharmaceutical compositions that antagonize the activity of the gene product of interest.

In certain embodiments, antisense is used to decrease expression of a gene. Antisense is used in reference to RNA sequences that are complementary to a specific RNA sequence (e.g., mRNA). Included within antisense are antisense RNA (“asRNA”) molecules involved in gene regulation by bacteria. Antisense RNA may be produced by any method, including synthesis by splicing the gene(s) of interest in a reverse orientation to a viral promoter that permits the synthesis of a coding strand. Once introduced, this transcribed strand combines with natural mRNA to form duplexes. These duplexes then block either the further transcription of the mRNA or its translation. Regions of a nucleic acid sequences that are accessible to antisense molecules can be determined using available computer analysis methods.

In certain embodiments, an RNA interference (RNAi) molecule is used to decrease expression of a gene. RNA interference (RNAi) is defined as the ability of double-stranded RNA (dsRNA) to suppress the expression of a gene corresponding to its own sequence. RNAi is also called post-transcriptional gene silencing or PTGS. Since the only RNA molecules normally found in the cytoplasm of a cell are molecules of single-stranded mRNA, the cell has enzymes that recognize and cut dsRNA into fragments containing 21-25 base pairs (approximately two turns of a double helix). The antisense strand of the fragment separates enough from the sense strand so that it hybridizes with the complementary sense sequence on a molecule of endogenous cellular mRNA. This hybridization triggers cutting of the mRNA in the double-stranded region, thus destroying its ability to be translated into a polypeptide. Introducing dsRNA corresponding to a particular gene thus knocks out the cell's own expression of that gene in particular tissues and/or at a chosen time.

Double-stranded RNA can be used to interfere with gene expression in mammals (Wianny & Zernicka-Goetz, 2000, Nature Cell Biology 2: 70-75; incorporated herein by reference in its entirety). dsRNA is used as inhibitory RNA or RNAi of the function of the gene of interest to produce a phenotype that is the same as that of a null mutant of the gene of interest (Wianny & Zernicka-Goetz, 2000, Nature Cell Biology).

In another embodiment, a method is provided which comprises screening compounds for their ability to increase or decrease the activity of one or more of the targets identified according to the methods of the invention. Accordingly, an assay is provided which comprises measuring gene expression (pre-symptomatic or symptomatic) in an animal model of schizophrenia at relevant time points previously determined, and comparing it to the same time points in control animals, or to earlier time points within the same animals, to establish dysregulation. Once these targets are identified as described by the methods herein as being relevant for schizophrenia or the symptoms thereof, they can be utilized in order to screen for compounds that modulate the gene targets or products thereof.

In some embodiments, a test compound, which is any compound of interest, is administered to animals displaying dysregulation of the targets that have been identified, and in some embodiments, the compound is also administered to control animals. It is then determined if the compound has any influence on the gene expression or the products thereof. Concurrent with this, symptoms of schizophrenia, and other behavioral and biological measures can be assessed from those animals in order to more fully elucidate the effects that the compound has on gene expression relevant to schizophrenia, and on the behavioral and biological manifestations of schizophrenia or the symptoms thereof. Compounds that bind to the gene products either competitively or non-competitively, or otherwise affect their activity may be useful as drug candidates for preventing or treating schizophrenia or the symptoms thereof. For example, if utilizing the social isolation rearing model in rats, compounds that can counteract the dysregulation of gene expression or products thereof at postnatal day 60, by, for example, reducing mRNA of GABAARα4 complexin 1, synapsin 2, or parvalbumin, or SEQ ID NO: 1 through SEQ ID NO: 10, may be useful for modulating, preventing, or treating schizophrenia or the symptoms thereof.

In another embodiment of the invention, a method is provided of preventing or treating schizophrenia, or the symptoms thereof, or the cognitive deficits associated with schizophrenia by administering to an individual in need thereof an effective amount of midkine. As described herein, cognitive abnormalities are a core symptom of schizophrenia, which can be manifest in numerous ways, and are not the result of medication. All cognitive deficits are within the scope of the invention, including all variations of abnormalities in learning; abnormalities in any kind of memory, including but not limited to short term memory, long term memory, episodic memory, working memory, declarative (explicit) and procedural (implicit) memory, semantic memory, spatial memory, visuospatial memory, memory consolidation, memory re-consolidation; as well as abnormalities in thought, verbal skills, language processing; as well reduced mental speed and reduced reaction time; thought disorder; problems with planning and complex sentences; difficulty generating novel strategies to solve a problem; and the failure to show mental flexibility.

As described herein, prepulse inhibition is used as a readout of sensorimotor processing in the prefrontal cortex, and is closely tied with working memory function, and as demonstrated herein, is greatly reduced in an animal model of schizophrenia. As demonstrated in the Examples herein, administration of midkine into the 3rd cerebral ventricle of the brain reverses the deficit observed in prepulse inhibition of startle in animals that have been reared in isolation.

Pharmaceutical compositions comprising the genes or gene fragments derived according to the methods of the invention, or comprising one or more of the genes and gene fragments or their products as described herein are contemplated by the invention. Also within the scope of the invention are pharmaceutical compositions comprising agonists and antagonists of one or more of the genes, fragments, or products thereof, which are useful in the prevention or treatment of schizophrenia or the symptoms thereof.

The pharmaceutical compositions of the invention can be formulated as neutral or salt forms. Pharmaceutically acceptable non-toxic salts include the base addition salts (formed with free carboxyl or other anionic groups) which may be derived from inorganic bases such as, for example, sodium, potassium, ammonium, calcium, or ferric hydroxides, and such organic bases as isopropylamine, trimethylamine, 2-ethylamino-ethanol, histidine, procaine, and the like. Such salts may also be formed as acid addition salts with any free cationic groups and will generally be formed with inorganic acids such as, for example, hydrochloric, sulfuric, or phosphoric acids, or organic acids such as acetic, p-toluenesulfonic, methanesulfonic acid, oxalic, tartaric, mandelic, and the like. Salts of the invention include amine salts formed by the protonation of an amino group with inorganic acids such as hydrochloric acid, hydrobromic acid, hydroiodic acid, sulfuric acid, phosphoric acid, and the like. Salts of the invention also include amine salts formed by the protonation of an amino group with suitable organic acids, such as p-toluenesulfonic acid, acetic acid, and the like. Additional excipients which are contemplated for use in the practice of the present invention are those available to those of ordinary skill in the art, for example, those found in the United States Pharmacopeia Vol. XXII and National Formulary Vol. XVII, U.S. Pharmacopcia Convention, Inc., Rockville, Md. (1989), the relevant contents of which are incorporated herein by reference.

Pharmaceutically acceptable carriers for use in the invention can be determined in part by the specific composition administered, as well as by the particular method used to administer the composition. Accordingly, there are a wide variety of suitable formulations of pharmaceutical compositions of the present invention (see, e.g., Remington's Pharmaceutical Sciences, 20th ed., 2003).

The term “pharmaceutically acceptable” can mean approved by a regulatory agency of the Federal or a state government or listed in the U.S. Pharmacopeia or other generally recognized pharmacopeia for use in animals, and for use in humans. The term “carrier” can mean a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Saline is a preferred carrier when the pharmaceutical composition is administered intravenously. Aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective amount of a prophylactic or therapeutic agent preferably in purified form, together with a suitable amount of carrier so as to provide the form for proper administration to the patient. The formulation should suit the mode of administration. In a preferred embodiment, the pharmaceutical compositions are sterile and in suitable form for administration to a subject, preferably an animal subject, more preferably a mammalian subject, and most preferably a human subject.

In another embodiment, the composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, pp. 317-327).

In yet another embodiment of the invention, the composition can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the nucleic acids of the invention or fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. No. 5,679,377; U.S. Pat. No. 5,916,597; U.S. Pat. No. 5,912,015; U.S. Pat. No. 5,989,463; U.S. Pat. No. 5,128,326; International Publication No. WO 99/15154; and International Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the therapeutic target, i.e., the lungs, thus requiring only a fraction of the systemic dose (see, e.g., Goodson, in Medical Applications of Controlled Release, supra, vol. 2, pp. 115-138 (1984)).

Controlled release systems are discussed in Langer (1990, Science 249:1527-1533). Any technique known to one of skill in the art can be used to produce sustained release formulations comprising one or more genes of the invention or fragments thereof. See, e.g., U.S. Pat. No. 4,526,938, International publication No. WO 91/05548, International publication No. WO 96/20698, Ning et al., 1996, “Intratumoral Radioimmunotheraphy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entirety.

In some embodiments of the invention, in which the composition is one or more nucleic acid molecules obtained according to the methods of the invention as described herein, the nucleic acid or nucleic acids can be administered in vivo by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression by homologous recombination.

Pharmaceutical compositions of the invention are formulated to be compatible with their intended route of administration. Examples of suitable routes of administration include, but are not limited to, parenteral (e.g., intravenous, intramuscular, intradermal, intra-tumoral, intra-synovial, and subcutaneous), oral (e.g., inhalation), intranasal, transdermal (topical), transmucosal, intra-tumoral, intra-synovial, vaginal, and rectal administration. In a specific embodiment, the composition is formulated in accordance with routine procedures as a pharmaceutical composition adapted for intravenous, subcutaneous, intramuscular, oral, intra-tummoral, intra synnovial, intranasal or topical administration to human beings. Typically, compositions for intravenous administration are solutions in sterile isotonic aqueous buffer. Where necessary, the composition may also include a solubilizing agent and a local anesthetic such as lignocaine to ease pain at the site of the injection.

If the compositions of the invention are to be administered orally, the compositions can be formulated orally in the form of, e.g., gum, tablets, capsules, cachets, gelcaps, solutions, suspensions and the like. Tablets or capsules can be prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated for slow release, controlled release or sustained release of a prophylactic or therapeutic agent(s).

If the compositions of the invention are to be administered intranasally, the compositions can be formulated in an aerosol form, spray, mist or in the form of drops. In particular, prophylactic or therapeutic agents for use according to the present invention can be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compositions of the invention may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

If the compositions of the invention are to be administered topically, the compositions can be formulated in the form of, e.g., an, ointment, cream, transdermal patch, lotion, gel, oral gel, spray, aerosol, solution, emulsion, or other form well-known to one of skill in the art. See, e.g., Remington's Pharmaceutical Sciences and Introduction to Pharmaceutical Dosage Forms, 4.sup.th ed., Lea & Febiger, Philadelphia, Pa. (1985). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well-known in the art.

In addition to the formulations described above, the compositions of the invention may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt.

Generally, the ingredients of compositions of the invention, such as nucleic acids or proteins are supplied either separately or mixed together in unit dosage form, for example, as a dry lyophilized powder or water free concentrate in a hermetically sealed container such as an ampoule or sachette indicating the quantity of active agent. If the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### EXAMPLES

It is understood that the following examples and embodiments described herein are for illustrative purposes only and that various modifications or changes in light thereof will be suggestive to persons skilled in the art and are to be included within the spirit and purview of this application and the scope of the appended claims. All publications, patents, and patent applications cited herein are hereby incorporated by reference in their entirety for all purposes.

Unless otherwise specified, in the examples provided below, animals were maintained as follows.

Animal Maintenance

Postnatal day 80 male Wistar rats (300-350 g) were obtained from the Biomedical Facility at University College Dublin and were group housed on a 12:12 light/dark cycle, with ad libitum access to food and water. The animals were introduced into the experimental rooms 5 days prior to commencement of training. On the 2 days prior to training the animals were handled, their weights monitored and spontaneous behavior assessed in an open field apparatus (620 mm long, 620 mm wide, 150 mm high). The floor of the open field was ruled into a series of squares (77×77 mm), the animal was placed in the centre and the number of lines crossed in a 5-minute period counted. Other behaviors assessed included rearing, grooming, piloerection, defecation and posture. All observations were carried out in the quiet room under low-level red illumination between 08:00 and 12:00 to minimize circadian influence. Naïve littermates were treated precisely as their trained counterparts except they were not exposed to the training environment prior to sacrifice.

### Example 1

### Social Isolation Rearing Leads to Deficits in Sensorimotor Gating

Materials and Methods

Isolation Rearing

Isolation-reared animals (isolated animals) were housed singly in non-soft bottom cages (22.5 cm×34.5 cm×17 cm), from time of weaning (postnatal day 25, P25) until completion of behavioral testing. A standard 12 hour light/dark cycle was observed and food and water was provided ad libitum. Noise and visual stimuli were kept to an absolute minimum as described previously by Geyer et al., 1993. Socially reared animals (social animals) were housed in groups of 4 from time of weaning until surgical implantation of a guide cannula on P80, there after they were housed singly. Soft bottom cages were used (27.5 cm×40.5 cm×20.1 cm), a standard 12 hour light/dark cycle observed and food and water provided ad libitum. The cages were floored with sawdust and contained a single open-ended cardboard cylinder for environmental enrichment.

Maternal Deprivation

The maternal deprivation protocol employed was as described previously (Ellenbroek et al 1998). Briefly, rat pups were isolated from their mothers for a single 24-hour period on P9. The dams were housed in the same room as the pups but in a separate cage. At the end of the 24-hour period the dams were returned to their litters and left undisturbed until weaning on P25. After weaning on P25 pups were housed in groups of four per cage. Social control animals were reared in groups of four under standard conditions. All animals were housed singly from P80, just prior to behavioral manipulation and/or surgery.

Prepulse Inhibition of Startle Response (Ellenbroek et al., 1998)

The startle apparatus consisted of a sound attenuating chamber (54.6×50.8×30.5 cm), a startle platform which measured the startle response, and an audio stimulator, controlled by the startle reflex software (MED Associates Inc.). Animals were restrained in an appropriately sized animal holder, placed on the startle platform. Animals were placed in the apparatus and allowed to acclimatize to a background noise of 70 dB[A] for 5 minutes. Subsequently, the animals received five startle trials (120bD[A] burst of white noise lasting 20 ms). Next, the rats received five blocks of trials, consisting of two startle trials and four prepulse inhibition trials (of differing prepulse intensity), as well as a no stimulus condition. The prepulse inhibition trials consisted of a prepulse stimulus of 72, 76, 80 or 84 dB[A] burst of white noise, followed 100 ms later by the startle stimulus of 120 dB[A]. The inter-trial interval was between 10 and 20 s. The session terminated with five further startle trials. The resulting movement of the rats was measured during 100 ms after startle stimulus onset, and then rectified, amplified and analyzed by computer and the maximal response and average response over the 100 ms period determined. Baseline startle amplitude was determined as the mean response of all startle trials. The percent prepulse inhibition was determined according to the following formula: 100-([startle amplitude at prepulse trial/startle amplitude at startle trial]×100%). The effect on baseline startle amplitude was analyzed by a one-way analysis of variance (ANOVA). The effect of rearing on PPI was determined by 2-way ANOVA (factors: rearing and prepulse intensity), with post-hoc Bonferroni tests.

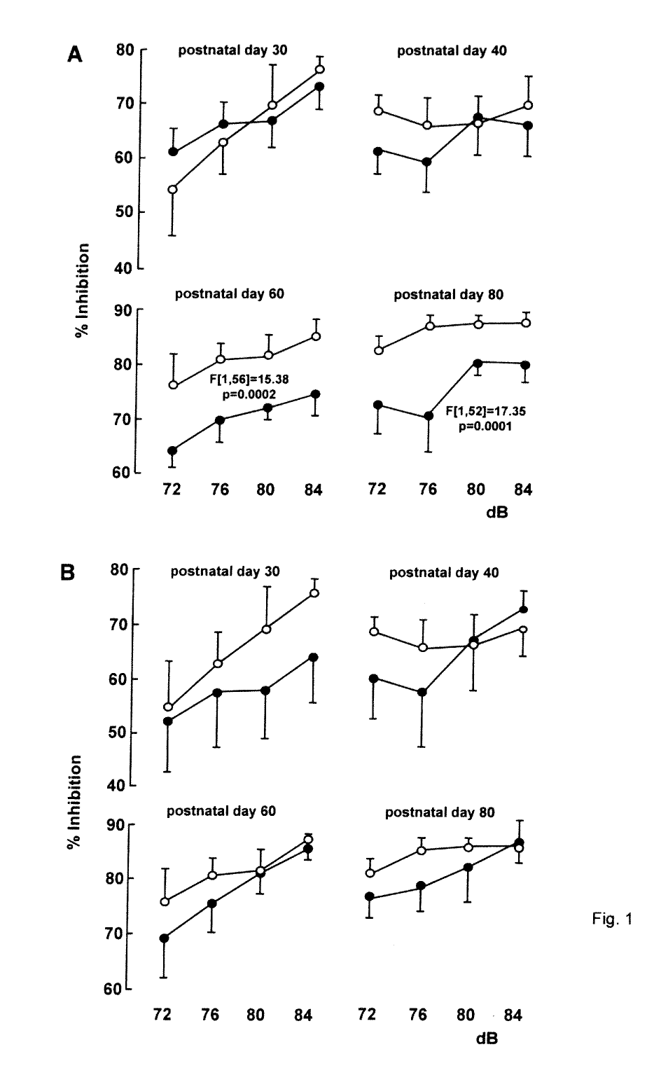

Separate cohorts of animals were analyzed at P30, P40, P60 and P80 (P=postnatal day). All isolation reared animals were compared to age-matched social controls and all maternally deprived animals were compared to age-matched non-deprived controls (raised in cages of 3-4 from weaning, n=8/group). Isolated animals exhibited impaired sensorimotor gating as measured by prepulse inhibition, when analyzed at P60, and this deficit was maintained at P80 (FIG. 1A).

### Example 2

### Social Isolation Rearing and Maternal Deprivation Lead to Deficits in Spatial Memory

Materials and Methods

Isolation reared animals, maternally deprived animals, socially reared controls, and non-deprived controls were maintained as described in Example 1.

Water Maze Training

The spatial learning task has been described in detail previously (Murphy et al., 1996). The water maze apparatus consisted of a large circular pool (1 m diameter, 80 cm high, temperature 26±1° C.) with a platform (11 cm diameter) submerged 1.5 cm below the water surface. Both the pool and the platform were constructed of black polyvinyl plastic and offered no intra-maze cues to guide escape behavior. The experimental room contained several extra-maze visual cues. During training the platform was hidden in the same quadrant 30 cm from the edge of the maze. Each trial started with the rat facing the wall of the maze at one of three locations. The time taken by the rat to find the hidden platform within a 60 sec period was recorded. On the first trial, rats failing to find the platform within the 60 sec period were placed on it for 10 sec. Times to the platform were measured over 5 trials in the training session with an inter-trial interval of 300 sec. To control for stress and other non-learning associated factors during water maze training each trained animal was paired with a corresponding passive control animal that was allowed to swim in the maze for a time matching its trained counterpart for each trial but in the absence of a platform.

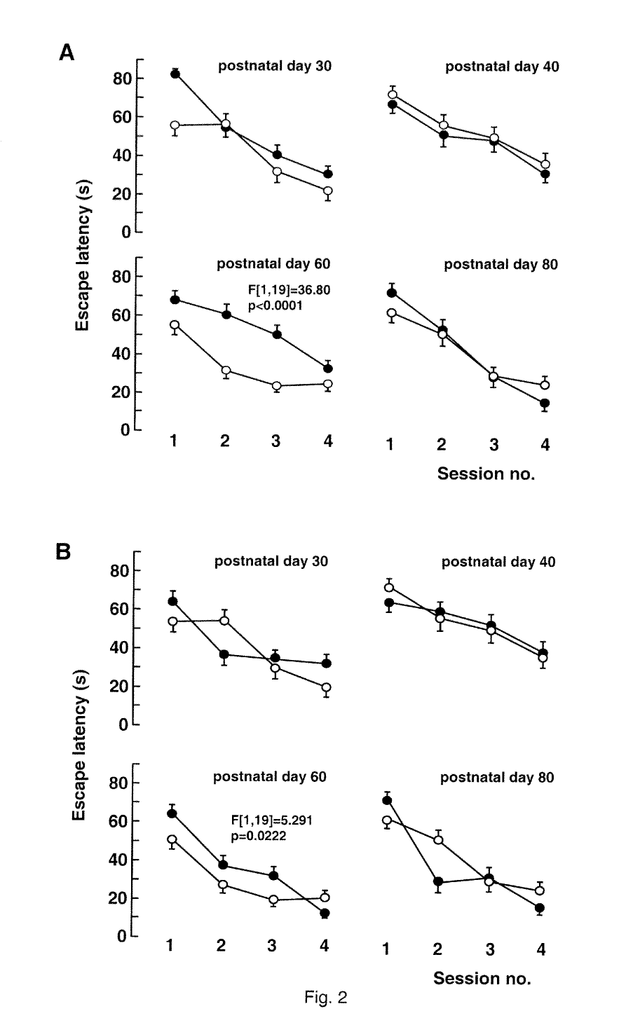

Separate cohorts were analyzed at P30, P40, P60 and P80. All isolation reared animals were compared to age-matched social controls and all maternally deprived animals were compared to age-matched non-deprived controls (raised in cages of 3-4 from weaning, n=8/group). Socially isolated animals and maternally deprived exhibited impaired spatial learning when analyzed at P60 in the Water Maze task (FIG. 2).

### Example 3

### mRNA Expression Profiles in Prefrontal Cortex (PFC) in Socially Isolated Animals and Maternally Deprived Animals

Materials and Methods

Isolation reared animals, maternally deprived animals, socially reared controls, and non-deprived controls were maintained as described in Example 1.

Sample Collection

In order to determine schizophrenia-associated developmental alterations of mRNA expression in the rat medial prefrontal cortex, this brain region was dissected from postnatal day 30, 40, 60 and 80 in isolation reared animals, socially housed control counterparts, maternally deprived animals, and non-deprived counterparts. Animals were killed by cervical dislocation, the medial prefrontal cortex rapidly dissected and snap frozen. All experimental procedures were approved by the Animal Research Ethics Committee of the Biomedical Facility at University College, Dublin, and were carried out by individuals who held the appropriate license issued by the Minister for Health and Children.

Microarray and Real-Time Sample Preparation

Total RNA was extracted from each medial prefrontal cortex by homogenisation in TRIzol reagent (Invitrogen, Carlsbad, Calif., USA) and following the TRIzol protocol. The resulting RNA samples were purified using an RNeasy mini kit (Qiagen, UK). RNA concentration was determined spectrophotmetrically, and RNA integrity was confirmed by agarose gel electrophoresis. Double-stranded cDNA was synthesised from 10 μg total RNA (Superscript System, Invitrogen, Carlsbad, Calif., USA). Briefly, the RNA was mixed with 100 pmol oligonucleotide GGCCATGGAATTGTAATACGACTCACTATAGGGAGGCGG (dT)24 in 20 μl water, annealed at 70° C. for 10 min, and quick-chilled. Buffer, dithiothereitol, and dNTP mix were than added and incubated at 37° C. for 2 min. Second-strand synthesis was performed by adding reaction buffer, dNTPs (200M), DNA ligase (10 U), DNA polymerase (40 U), ribonuclease H (2 U), and water (to a final volume of 150 μl), and the reaction was incubated for 2 h at 16° C. This was followed by addition of 10 U T4 DNA polymerase and incubation at 16° C. for 5 min. The cDNA was purified by phenol/chloroform extraction, precipitated, and transcribed in vitro using T7 RNA polymerase. Biotinylated cRNA was generated using the BioArray HighYield RNA Transcription Kit (Enzo Diagnostics, Inc., Farmingdale, N.Y.). The cRNA was purified by RNeasy minispin columns and fragmented by incubation in 40 mM Tris (pH 8.1), 100 mM potassium acetate, and 30 mM magnesium acetate buffer at 94° C. for 35 min.

Microarray Analysis

Fragmented cRNA for each sample was hybridised to the Affymetrix rat genome RG230.02 chip using the protocol outlined in the GeneChip Expression Analysis Technical Manual (Affymetrix Inc., Santa Clara, Calif., USA). Hybridized chips were washed and stained using Affymetrix Fluidics Station 400 and EukGE-WS1 Standard Format as recommended by the manufacturer. The staining was performed using streptacidin-phycoerythrin conjugate (SAPE; Molecular Probes, Eugene, Oreg., USA), followed by biotinylated antibody against streptacidin (Vector Laboratiories, Burlingame, Calif., USA), and then SAPE. The chips were scanned using a Hewlett-Packard GeneArray Scanner and analyzed using Affymetrix MASS 0.0 software. Hybridization intensities were normalized using a method featuring a pool of 11 biotin-labeled cRNA control transcripts, derived by in vitro transcription of 11 cloned Bacillus subtilis genes, which were spiked into each hybridization experiment. This normalization method has been described in detail previously (Hill et al., 2001). The 5′/3′ ratio for glyceraldehydes-3-phosphate dehydrogenase (GAPDH) and for beta-actin ranged from 0.8 to 1.1.

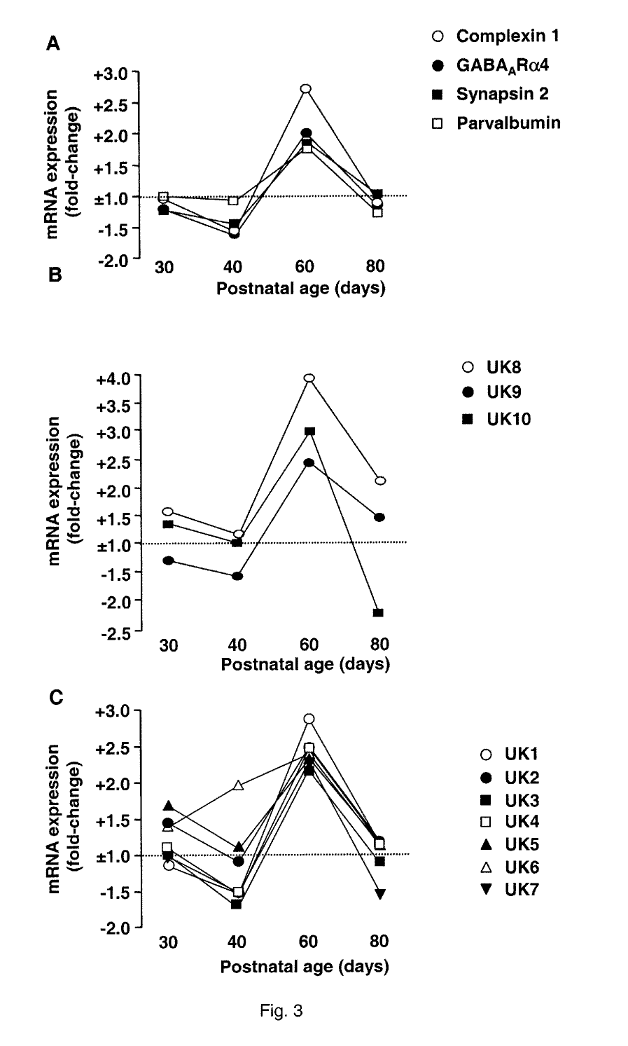

DNA microarrays were used to study the mRNA expression profiles of rat prefrontal cortex over time following social isolation rearing. The prefrontal cortex is the area of the brain attributed to processing deficits associated with schizophrenia. Identification of time for emergence of sensory processing and cognition deficits in the animal models of isolation rearing allowed analysis of transcriptional change in the presymptomatic (postnatal days 30 and 40) and symptomatic periods. Over the developmental timeframe investigated, in excess of 2000 genes were observed to change significantly in animals maintained in isolation from time of weaning A transient increase in 600-700 transcripts was observed at postnatal 60, the so-called ‘P60 spike’ (FIG. 3 represents a fraction of these transcripts).

The P60 spike was associated with a substantial number of unknown ESTs that exhibited the same significant elevations in transcription at this postnatal age as was observed with genes known to be associated with schizophrenia (FIG. 3). Analysis of the known genes in this core group revealed several transcripts of interest with respect to a potential role in schizophrenia signaling cascades, such as GABAA receptor alpha4 and complexin I, as well as parvalbumin (FIG. 3). These transcripts shared a characteristic temporal pattern of regulation following social isolation rearing, with decreases in expression at P40, and sharp increases in expression at P60 (FIG. 3).

This distinctive pattern of transcript modulation was used to develop a ‘seed pattern’ with which the entire transcriptional data set could be interrogated. This has generated a unique database of ESTs likely to have a significant role in schizophrenia. Collectively, these genes can be considered a core transcriptional program for schizophrenia. The potential of the unknown genes as targets for regulating schizophrenia and its symptoms is underpinned by the validation of the co-regulating known genes at message, protein and functional levels. Information about the unknown gene transcripts is provided in Table 1.

TABLE 1

| Code | Affy ID | Gene Name |
|---|---|---|
| UK1 | 1390262_a_at | DnaJ (Hsp40) homolog, subfamily C, |
| | | member 9 (predicted) |
| UK2 | 1384043_at | Transcribed locus |
| UK3 | 1376245_x_at | DnaJ (Hsp40) homolog, subfamily C, |
| | | member 9 (predicted) |
| UK4 | 1375443_at | Similar to gene trap ROSA b-geo 22 |
| UK5 | 1381054_at | Similar to KIAA0980 protein (predicted) |
| UK6 | 1382299_at | Transcribed locus |
| UK7 | 1380870_at | Similar to RIKEN cDNA 1700040L02 |
| | | (predicted) |
| UK8 | 1375648_at | Similar to hypothetical protein (predicted) |
| UK9 | 1391249_at | Similar to DD1 (predicted) |
| UK10 | 1377448_at | Similar to RIKEN cDNA 1110001E17 |
| | | (predicted) |

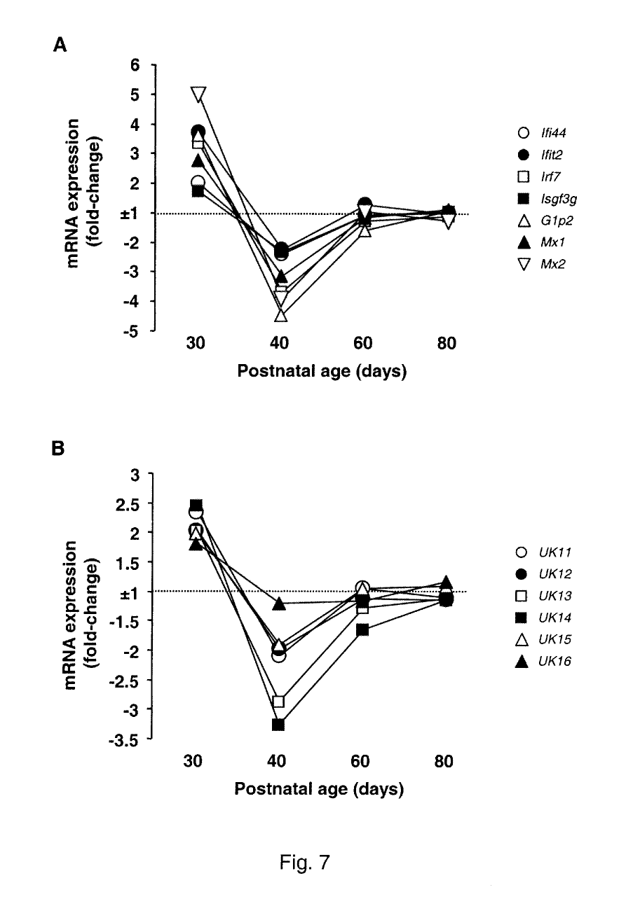

The P30 spike was associated with a substantial number of unknown ESTs that exhibited the same significant elevations in transcription at this postnatal age as was observed with genes known to be associated with an anti-viral response (FIG. 7). Analysis of the known genes in this core group revealed several transcripts of interest with respect to interferon signaling, including interferon regulatory factor 7, PKR, Ifi44, Ifit2, Irf7, Isgf3g, Glp2. Mx1, Mx2, and SEQ ID NO: 11 through SEQ ID NO: 16 (FIG. 7). These transcripts shared a characteristic temporal pattern of regulation following maternal deprivation, with increases in expression at P30, and sharp decreases in expression at P40 (FIG. 7).

Collectively, these genes can be considered a core transcriptional program for schizophrenia. The potential of the unknown genes as targets for regulating schizophrenia and its symptoms is underpinned by the validation of the co-regulating known genes at message, protein and functional levels. Information about the unknown gene transcripts is provided in Table 2.

TABLE 2

| Code | Affy ID | Gene Name |
|---|---|---|
| UK11 | 1391754_at | (2′,5′-oligoadenylate synthetase 1, 40/46 kDa, 2′-5′ |
| | | oligoadenylate synthetase 1F, 2′-5′ oligoadenylate |
| | | synthetase 1l) |
| UK12 | 1370913_at | (Best5 protein, hypothetical gene supported |
| | | by NM_138881) |
| UK13 | 1372604_at | (hypothetical protein LOC503164, similar |
| | | to RIKEN cDNA 2210421G13) |
| UK14 | 1379285_at | similar to 5830458K16Rik protein (predicted) |
| UK15 | 1397304_at | Transcribed locus |
| UK16 | 1378082_at | Transcribed locus |
| Ifi44 | 1381014_at | interferon-induced protein 44 (predicted) |
| Ifit2 | 1379568_at | interferon-induced protein with tetratricopeptide |
| | | repeats 2 (predicted) |
| Irf7 | 1383564_at | interferon regulatory factor 7 (predicted) |
| Isgf3g | 1374627_at | interferon dependent positive acting |
| | | transcription factor 3 gamma (predicted) |
| G1p2 | 1382314_at | interferon, alpha-inducible protein |
| | | (clone IFI-15K) (predicted) |
| Mx1 | 1371015_at | myxovirus (influenza virus) resistance 1 |
| Mx2 | 1369202_at | myxovirus (influenza virus) resistance 2 |

Genes and gene fragments identified as changing significantly in animals that were reared in social isolation, relative to social control animals on postnatal day 30 (P30) are provided in Table 3.

TABLE 3

| Fragment Name | Gene Name | Gene Symbol |
|---|---|---|
| 1394490_at | (ATP-binding cassette, sub-family A (ABC1), member 1, hypothetical gene | (Abca1, LOC497803) |
| | supported by NM_178095) | |
| 1392108_at | (ATP-binding cassette, sub-family C (CFTR/MRP), member 3, aldehyde oxidase 3, | (Abcc3, Aox3, CcI9_predicted, |
| | chemokine (C-C motif) ligand 9 (predicted), enoyl-Coenzyme A, hydratase/3- | Ehhadh, Gmpr2, Tnfrsf1b) |
| | hydroxyacyl Coenzyme A dehydrogenase, guanosine monophosphate reductase | |
| | 2, tumor necrosis factor recept | |
| 1387210_at | (discs, large homolog 4 (Drosophila), hypothetical gene supported by NM_019621) | (Dlgh4, LOC497670) |
| 1369471_at | (LOC499775, rapostlin) | (Fnbp1, LOC499775) |
| 1370454_at | (HS1 binding protein, homer homolog 1 (Drosophila)) | (Homer1, Hs1bp1) |
| 1370997_at | (HS1 binding protein, homer homolog 1 (Drosophila)) | (Homer1, Hs1bp1) |
| 1382331_at | (similar to RIKEN cDNA 0610038L10 gene, similar to riboflavin kinase) | (LOC317214, LOC499328) |
| 1389734_x_at | (MHC class I RT1.O type 149 processed pseudogene, RT1 class I, T24, gene 4, | (LOC360231, LOC499402, RT1- |
| | RT1 class Ib, locus H2-TL-like (S2), RT1 class Ib, locus S3, similar to class I | 149, RT1-S2, RT1-S3) |
| | histocompatibility antigen alpha chain - cotton-top tamarin) | |
| 1370428_x_at | (RT1 class I, A3, RT1 class I, CE10, RT1 class I, CE3, RT1 class I, CE7, RT1 | (RT1-A2, RT1-A3, RT1-Aw2, |
| | class Ia, locus A2, RT1 class Ib, locus Aw2) | RT1-CE10, RT1-CE3, RT1- |
| | | CE7) |
| 1390706_at | (short form of beta II spectrin, spectrin beta 2) | (Spnb1, Spnb2) |
| 1380577_at | ATP-binding cassette, sub-family G (WHITE), member 2 | Abcg2 |
| 1398836_s_at | actin, beta | Actb |
| 1374419_at | adenylate cyclase 9 (predicted) | Adcy9_predicted |
| 1369326_at | A kinase (PRKA) anchor protein 6 | Akap6 |
| 1369063_at | acidic (leucine-rich) nuclear phosphoprotein 32 family, member A | Anp32a |
| 1387068_at | activity regulated cytoskeletal-associated protein | Arc |
| 1391658_at | archain | Arcn1 |
| 1380525_at | arylsulfatase E (chondrodysplasia punctata 1) (predicted) | Arse_predicted |
| 1387234_at | alpha-2-glycoprotein 1, zinc | Azgp1 |
| 1391293_at | butyrobetaine (gamma), 2-oxoglutarate dioxygenase 1 (gamma-butyrobetaine | Bbox1 |
| | hydroxylase) | |
| 1369733_at | catenin beta | Catnb |
| 1388026_at | CD3 antigen, zeta polypeptide | Cd3z |
| 1371278_at | cell division cycle 34 homolog (S. cerevisiae) (predicted) | Cdc34_predicted |
| 1397673_at | CUG triplet repeat, RNA-binding protein 2 | Cugbp2 |
| 1375231_a_at | CXXC finger 5 | Cxxc5 |
| 1371142_at | cytochrome P450, subfamily 2G, polypeptide 1 | Cyp2g1 |
| 1385871_at | DEAH (Asp-Glu-Ala-His) box polypeptide 36 (predicted) | Dhx36_predicted |
| 1391602_at | developmentally regulated GTP binding protein 1 (predicted) | Drg1_predicted |
| 1368146_at | dual specificity phosphatase 1 | Dusp1 |
| 1368321_at | early growth response 1 | Egr1 |
| 1387306_a_at | early growth response 2 | Egr2 |
| 1387442_at | early growth response 4 | Egr4 |
| 1375043_at | FBJ murine osteosarcoma viral oncogene homolog | Fos |
| 1390686_at | gamma-aminobutyric acid (GABA) A receptor, subunit gamma 3 | Gabrg3 |
| 1368074_at | galactose-4-epimerase, UDP | Gale |
| 1379926_at | guanine nucleotide binding protein, alpha 12 | Gna12 |
| 1388085_at | glutathione peroxidase 6 | Gpx6 |
| 1376412_at | glutamate receptor, ionotropic, 4 | Gria4 |
| 1379546_at | hematological and neurological expressed sequence 1 | Hn1 |
| 1383564_at | interferon regulatory factor 7 (predicted) | Irf7_predicted |
| 1376845_at | putative ISG12(b) protein | isg12(b) |
| 1382535_at | potassium channel tetramerisation domain containing 12 (predicted) | Kctd12_predicted |
| 1384608_at | RN protein | LOC171116 |
| 1391426_a_at | similar to PEST-containing nuclear protein | LOC288165 |
| 1393436_at | similar to potential ligand-binding protein | LOC309100 |
| 1399073_at | similar to HSPC263 | LOC314660 |
| 1385832_s_at | similar to RIKEN cDNA 1200013B08 | LOC317578 |
| 1389270_x_at | similar to ATPase inhibitor | LOC497829 |
| 1374328_at | similar to hypothetical protein | LOC499213 |
| 1385656_at | similar to hypothetical protein MGC13034 | LOC499516 |
| 1376792_at | similar to CDNA sequence BC014699 | LOC500221 |
| 1375073_at | similar to sterile alpha motif domain containing 11 | LOC500601 |
| 1391595_at | similar to ribosomal protein L21 | LOC503211 |
| 1380177_at | methionine adenosyltransferase II, alpha | Mat2a |
| 1379333_at | similar to ALEX3 protein; arm protein lost in epithelial cancers, X chromosome, 3 | MGC93796 |
| 1369623_at | morphine related protein-1 (RefSeq = NM_022613) | Morp1 |
| 1375180_at | MARVEL (membrane-associating) domain containing 1 (predicted) | Mrvldc1_predicted |
| 1370783_a_at | membrane-spanning 4-domains, subfamily A, member 2 | Ms4a2 |
| 1385125_at | transcription factor myocardin | Myocd |
| 1386935_at | nuclear receptor subfamily 4, group A, member 1 | Nr4a1 |
| 1368958_at | protein kinase C and casein kinase substrate in neurons 1 | Pacsin1 |
| 1377122_at | phosphodiesterase 1A, calmodulin-dependent | Pde1a |
| 1383294_at | prodynorphin | Pdyn |
| 1371250_at | platelet factor 4 | Pf4 |
| 1383037_at | polymerase (DNA-directed), delta interacting protein 2 (predicted) | Poldip2_predicted |
| 1375328_at | protease, serine, 21 | Prss21 |
| 1380604_at | pumilio 2 (Drosophila) (predicted) | Pum2_predicted |
| 1386900_at | ribosome associated membrane protein 4 | RAMP4 |
| 1369614_at | RAP2B, member of RAS oncogene family | Rap2b |
| 1378287_at | radixin | Rdx |
| 1375542_at | radixin | Rdx |
| 1391042_at | similar to Metal-response element-binding transcription factor 2 (Metal-response | RGD1304727_predicted |
| | element DNA-binding protein M96) (predicted) | |
| 1397194_at | similar to CG5554-PA (predicted) | RGD1305146_predicted |
| 1385426_at | similar to hypothetical protein FLJ20647 (predicted) | RGD1305326_predicted |
| 1398609_at | similar to Rnf37-pending protein (predicted) | RGD1305440_predicted |
| 1384553_at | similar to hypothetical protein FLJ13089 (predicted) | RGD1305685_predicted |
| 1374258_at | similar to hypothetical protein MGC13138 (predicted) | RGD1306928_predicted |
| 1375648_at | similar to hypothetical protein (predicted) | RGD1307966_predicted |
| 1389269_at | similar to RIKEN cDNA 4933433P14 gene (predicted) | RGD1308470_predicted |
| 1373814_at | similar to mKIAA1002 protein (predicted) | RGD1310066_predicted |
| 1391387_s_at | similar to RIKEN cDNA 0610039G24 gene (predicted) | RGD1310828_predicted |
| 1376152_at | similar to flavoprotein oxidoreductase MICAL2 (predicted) | RGD1311773_predicted |
| 1398854_at | ribosomal protein L24 | Rpl24 |
| 1375788_at | ribosomal protein L7 | Rpl7 |
| 1390919_at | splicing factor, arginine/serine-rich 8 (suppressor-of-white-apricot homolog, | Sfrs8_predicted |
| | Drosophila) (predicted) | |
| 1389949_at | sialyltransferase 7c | Siat7c |
| 1369020_at | solute carrier family 5 (sodium iodide symporter), member 5 | Slc5a5 |
| 1389067_at | solute carrier organic anion transporter family, member 4a1 | Slco4a1 |
| 1381394_at | PDZ protein Mrt1 | Snx27 |
| 1388396_at | serine/threonine kinase 25 (STE20 homolog, yeast) | Stk25 |
| 1397148_at | stomatin (Epb7.2)-like 3 (predicted) | Stoml3_predicted |
| 1387662_at | synaptotagmin 4 | Syt4 |
| 1387484_at | transforming growth factor, beta receptor 3 | Tgfbr3 |
| 1368650_at | TGFB inducible early growth response | Tieg |
| 1371029_at | tuberous sclerosis 2 | Tsc2 |
| 1367598_at | transthyretin | Ttr |
| 1369413_at | Unc4.1 homeobox (C. elegans) | Uncx4.1 |
| 1386909_a_at | voltage-dependent anion channel 1 | Vdac1 |
| 1396688_at | voltage-dependent anion channel 2 | Vdac2 |

Genes and gene fragments identified as changing significantly in animals that were reared in social isolation, relative to social control animals on postnatal day 40 (P40) are provided in Table 4.

TABLE 4

| Fragment Name | Gene Name | Gene Symbol |
|---|---|---|
| 1370464_at | (ATP-binding cassette, sub-family B (MDR/TAP), member 1, ATP-binding | (Abcb1, Abcb1a, Abcb4) |
| | cassette, sub-family B (MDR/TAP), member 1A, ATP-binding cassette, sub- | |
| | family B (MDR/TAP), member 4) | |
| 1392108_at | (ATP-binding cassette, sub-family C (CFTR/MRP), member 3, aldehyde | (Abcc3, Aox3, Ccl9_predicted, |
| | oxidase 3, chemokine (C-C motif) ligand 9 (predicted), enoyl-Coenzyme A, | Ehhadh, Gmpr2, Tnfrsf1b) |
| | hydratase/3-hydroxyacyl Coenzyme A dehydrogenase, guanosine | |
| | monophosphate reductase 2, tumor necrosis factor recept | |
| 1387840_at | (acid phosphatase 1, soluble, son of sevenless homolog 2 (Drosophila)) | (Acp1, Sos2) |
| 1383079_at | (apoptotic peptidase activating factor 1, ceroid-lipofuscinosis, neuronal 2, | (Apaf1, Ccr5, Cln2, Galm, |
| | chemokine (C-C) receptor 5, galactose mutarotase (aldose 1-epimerase), | Gcat_predicted, LOC501658, |
| | glycine C-acetyltransferase (2-amino-3-ketobutyrate-coenzyme A ligase) | Psmd9) |
| | (predicted), proteasome (prosome, ma | |
| 1391673_at | (Rho GTPase activating protein 20, hypothetical gene supported by | (Arhgap20, LOC497830) |
| | NM_213629) | |
| 1370913_at | (Best5 protein, hypothetical gene supported by NM_138881) | (Best5, LOC497812) |
| 1370892_at | (complement component 4, gene 2, complement component 4a) | (C4-2, C4a) |
| 1395412_at | (GPI-anchored membrane protein 1 (predicted), similar to GPI-anchored | (Gpiap1_predicted, |
| | membrane protein 1) | LOC362174) |
| 1371245_a_at | (beta-glo, hemoglobin beta chain complex) | (Hbb, MGC72973) |
| 1369868_at | (hypothetical gene supported by NM_053946, implantation-associated protein) | (Iag2, LOC497846) |
| 1387770_at | (interferon, alpha-inducible protein 27-like, putative ISG12(a) protein) | (Ifi27l, isg12(a)) |
| 1387995_a_at | (interferon induced transmembrane protein 2 (1-8D), interferon induced | (Ifitm2, Ifitm3) |
| | transmembrane protein 3) | |
| 1370801_at | (integrin, beta 5, similar to integrin beta-5) | (Itgb5, LOC498091) |
| 1381030_at | (similar to step II splicing factor SLU7; DNA segment, Chr 11, ERATO Doi 730, | (LOC303057, Slu7) |
| | expressed; DNA segment, Chr 3, Brigham & Womens Genetics 0878 | |
| | expressed, step II splicing factor SLU7 (S. cerevisiae)) | |
| 1398588_at | (similar to step II splicing factor SLU7; DNA segment, Chr 11, ERATO Doi 730, | (LOC303057, Slu7) |
| | expressed; DNA segment, Chr 3, Brigham & Womens Genetics 0878 | |
| | expressed, step II splicing factor SLU7 (S. cerevisiae)) | |
| 1397824_at | similar to WAC | (LOC307029, LOC361260) |
| 1372604_at | (hypothetical protein LOC503164, similar to RIKEN cDNA 2210421G13) | (LOC315106, LOC503164) |
| 1382331_at | (similar to RIKEN cDNA 0610038L10 gene, similar to riboflavin kinase) | (LOC317214, LOC499328) |
| 1388212_a_at | (MHC class I RT1.O type 149 processed pseudogene, RT1 class I, T24, gene | (LOC360231, LOC499402, |
| | 4, RT1 class Ib, locus H2-TL-like (S2), RT1 class Ib, locus S3, similar to class I | RT1-149, RT1-S2, RT1-S3) |
| | histocompatibility antigen alpha chain - cotton-top tamarin) | |
| 1389734_x_at | (MHC class I RT1.O type 149 processed pseudogene, RT1 class I, T24, gene | (LOC360231, LOC499402, |
| | 4, RT1 class Ib, locus H2-TL-like (S2), RT1 class Ib, locus S3, similar to class I | RT1-149, RT1-S2, RT1-S3) |
| | histocompatibility antigen alpha chain - cotton-top tamarin) | |
| 1388164_at | (MHC class I RT1.O type 149 processed pseudogene, RT1 class I, T24, gene | (LOC360231, LOC499402, |
| | 4, RT1 class Ib, locus H2-TL-like (S2), RT1 class Ib, locus S3, similar to class I | RT1-149, RT1-S2, RT1-S3) |
| | histocompatibility antigen alpha chain - cotton-top tamarin) | |
| 1371123_x_at | (MHC class I RT1.O type 149 processed pseudogene, RT1 class I, T24, gene | (LOC360231, LOC499402, |
| | 4, RT1 class Ib, locus H2-TL-like (S2), RT1 class Ib, locus S3, similar to class I | RT1-149, RT1-S2, RT1-S3) |
| | histocompatibility antigen alpha chain - cotton-top tamarin) | |
| 1388213_a_at | (MHC class I RT1.O type 149 processed pseudogene, RT1 class I, T24, gene | (LOC360231, LOC499402, |
| | 4, RT1 class Ib, locus H2-TL-like (S2), RT1 class Ib, locus S3, similar to class I | RT1-149, RT1-S2, RT1-S3) |
| | histocompatibility antigen alpha chain - cotton-top tamarin) | |
| 1370500_a_at | (hypothetical gene supported by NM_012720, myelin-associated | (LOC360443, Mobp) |
| | oligodendrocytic basic protein) | |
| 1377174_at | (SET binding factor 1 (predicted), similar to mKIAA3020 protein) | (LOC362985, Sbf1_predicted) |
| 1397522_at | (SET binding factor 1 (predicted), similar to mKIAA3020 protein) | (LOC362985, Sbf1_predicted) |
| 1370642_s_at | (hypothetical gene supported by NM_031525, platelet derived growth factor | (LOC497724, Pdgfrb) |
| | receptor, beta polypeptide) | |
| 1368150_at | (hypothetical gene supported by NM_031736, solute carrier family 27 (fatty | (LOC497779, Slc27a2) |
| | acid transporter), member 2) | |
| 1367823_at | (similar to testis specific protein, Ddc8, tissue inhibitor of metalloproteinase 2) | (LOC498028, Timp2) |
| 1373992_at | (similar to MGC108823 protein, similar to interferon-inducible GTPase) | (LOC498872, MGC108823) |
| 1372158_at | (LRP16 protein, similar to fibronectin leucine rich transmembrane protein 1) | (LOC499308, Lrp16) |
| 1379062_at | (similar to Expressed sequence AU019823, translocase of inner mitochondrial | (LOC500994, Timm8b) |
| | membrane 8 homolog b (yeast)) | |
| 1370081_a_at | (LOC501102, vascular endothelial growth factor A) | (LOC501102, Vegfa) |
| 1372327_at | (myelin basic protein expression factor 2, repressor (predicted), solute carrier | (Myef2_predicted, |
| | family 24, member 5 (predicted)) | Slc24a5_predicted) |
| 1388000_at | (potassium-dependent sodium-calcium exchanger, solute carrier family 24 | (NCKX2, Slc24a2) |
| | (sodium/potassium/calcium exchanger), member 2) | |
| 1391754_at | (2′,5′-oligoadenylate synthetase 1, 40/46 kDa, 2′-5′ oligoadenylate synthetase | (Oas1, Oas1f, Oas1i) |
| | 1F, 2′-5′oligoadenylate synthetase 1I) | |
| 1371152_a_at | (2′,5′-oligoadenylate synthetase 1, 40/46 kDa, 2′-5′ oligoadenylate synthetase | (Oas1, Oas1i, Oas1k) |
| | 1I, 2′-5′ oligoadenylate synthetase 1K) | |
| 1382943_at | (protocadherin gamma subfamily A, 10 (predicted), protocadherin gamma | (Pcdhga10_predicted, |
| | subfamily A, 11, protocadherin gamma subfamily A, 5 (predicted), | Pcdhga11, Pcdhga5_predicted, |
| | protocadherin gamma subfamily A, 6, protocadherin gamma subfamily A, 7 | Pcdhga6, Pcdhga7_predicted, |
| | (predicted), protocadherin gamma subfamily A, 8 ( | Pcdhga8_predicted, Pcdhga9, |
| | | Pcdhgb5_predicted, Pcdhgc3) |
| 1396211_at | (ROD1 regulator of differentiation 1 (S. pombe), similar to RIKEN cDNA | (RGD1305387_predicted, |
| | 2610207I16 (predicted)) | Rod1) |
| 1388071_x_at | (RT1 class I, A3, RT1 class I, CE10, RT1 class I, CE7, RT1 class Ib, locus | (RT1-A3, RT1-Aw2, RT1-CE10, |
| | Aw2) | RT1-CE7) |
| 1388153_at | acyl-CoA synthetase long-chain family member 1 | Acsl1 |
| 1398836_s_at | actin, beta | Actb |
| 1379687_at | ARP2 actin-related protein 2 homolog (yeast) (predicted) | Actr2_predicted |
| 1395886_at | ARP3 actin-related protein 3 homolog (yeast) | Actr3 |
| 1374419_at | adenylate cyclase 9 (predicted) | Adcy9_predicted |
| 1397516_at | asparagine-linked glycosylation 2 homolog (yeast, alpha-1,3- | Alg2_predicted |
| | mannosyltransferase) (predicted) | |
| 1387837_at | adenomatosis polyposis coli | Apc |
| 1395894_at | apoptosis inhibitor 5 (predicted) | Api5_predicted |
| 1380533_at | amyloid beta (A4) precursor protein | App |
| 1387068_at | activity regulated cytoskeletal-associated protein | Arc |
| 1398817_at | ADP-ribosylation factor 1 | Arf1 |
| 1368925_a_at | Rho guanine nucleotide exchange factor 7 | Arhgef7 |
| 1371108_a_at | ATPase, Na+/K+ transporting, alpha 1 polypeptide | Atp1a1 |
| 1369798_at | ATPase, Na+/K+ transporting, beta 2 polypeptide | Atp1b2 |
| 1377386_at | ATPase, Ca++-sequestering | Atp2c1 |
| 1389470_at | B-factor, properdin | Bf |
| 1386994_at | B-cell translocation gene 2, anti-proliferative | Btg2 |
| 1398251_a_at | calcium/calmodulin-dependent protein kinase II beta subunit | Camk2b |
| 1369993_at | calcium/calmodulin-dependent protein kinase II gamma | Camk2g |
| 1371687_at | calnexin | Canx |
| 1369733_at | catenin beta | Catnb |
| 1392937_at | cyclin I (predicted) | Ccni_predicted |
| 1368976_at | CD38 antigen | Cd38 |
| 1369025_at | CD5 antigen | Cd5 |
| 1387695_at | Cdc42-binding protein kinase beta | Cdc42bpb |
| 1375719_s_at | cadherin 13 | Cdh13 |
| 1369538_at | cyclin-dependent kinase 5, regulatory subunit 1 (p35) | Cdk5r |
| 1368449_at | centaurin, alpha 1 | Centa1 |
| 1380063_at | cholesterol 25-hydroxylase (predicted) | Ch25h_predicted |
| 1369239_at | chloride channel 5 | Clcn5 |
| 1369609_at | claudin 11 | Cldn11 |
| 1370693_a_at | cyclic nucleotide phosphodiesterase 1 | Cnp1 |
| 1370864_at | collagen, type 1, alpha 1 | Col1a1 |
| 1388116_at | collagen, type 1, alpha 1 | Col1a1 |
| 1370155_at | procollagen, type I, alpha 2 | Col1a2 |
| 1369811_at | complexin 1 | Cplx1 |
| 1369495_at | corticotropin releasing hormone receptor 2 | Crhr2 |
| 1383761_at | Chorionic somatomammotropin hormone 1 variant; Placental lactogen-1 | Csh1v |
| 1387879_a_at | CUG triplet repeat, RNA-binding protein 2 | Cugbp2 |
| 1369136_at | Cytochrome P450, subfamily IIA (phenobarbital-inducble)/(Cytochrome P450 | Cyp2a3a |
| | IIA3) | |
| 1368608_at | cytochrome P450, family 2, subfamily f, polypeptide 2 | Cyp2f2 |
| 1371142_at | cytochrome P450, subfamily 2G, polypeptide 1 | Cyp2g1 |
| 1370387_at | cytochrome P450, family 3, subfamily a, polypeptide 13 | Cyp3a13 |
| 1390738_at | DAMP-1 protein | Damp1 |
| 1391463_at | DEAD (Asp-Glu-Ala-Asp) box polypeptide 58 (predicted) | Ddx58_predicted |
| 1391406_at | degenerative spermatocyte homolog (Drosophila) | Degs |
| 1391602_at | developmentally regulated GTP binding protein 1 (predicted) | Drg1_predicted |
| 1395586_at | eukaryotic translation elongation factor 1 alpha 1 | Eef1a1 |
| 1387306_a_at | early growth response 2 | Egr2 |
| 1387442_at | early growth response 4 | Egr4 |
| 1397520_at | eukaryotic translation initiation factor 4 gamma, 2 | Eif4g2 |
| 1382710_at | ectodermal-neural cortex 1 | Enc1 |
| 1386907_at | enolase 3, beta | Eno3 |
| 1397826_at | erythrocyte protein band 4.9 (predicted) | Epb4.9_predicted |
| 1387715_at | extracellular peptidase inhibitor | Expi |
| 1382173_at | fibrillarin (predicted) | Fbl_predicted |
| 1393252_at | fibulin 1 (predicted) | Fbln1_predicted |
| 1387351_at | fibrillin 1 | Fbn1 |
| 1383354_a_at | F-box only protein 33 (predicted) | Fbxo33_predicted |
| 1392820_at | fibroblast growth factor 1 | Fgf1 |
| 1370747_at | fibroblast growth factor 9 | Fgf9 |
| 1390049_at | four and a half LIM domains 1 | Fhl1 |
| 1375043_at | FBJ murine osteosarcoma viral oncogene homolog | Fos |
| 1382314_at | interferon, alpha-inducible protein (clone IFI-15K) (predicted) | G1p2_predicted |
| 1369905_at | gamma-aminobutyric acid (GABA-A) receptor, subunit alpha 4 | Gabra4 |
| 1395269_s_at | gamma-aminobutyric acid A receptor, delta | Gabrd |
| 1388792_at | growth arrest and DNA-damage-inducible 45 gamma (predicted) | Gadd45g_predicted |
| 1368332_at | guanylate nucleotide binding protein 2 | Gbp2 |
| 1380230_at | GTP cyclohydrolase 1 | Gch |
| 1369640_at | gap junction membrane channel protein alpha 1 | Gja1 |
| 1367633_at | glutamine synthetase 1 | Glul |
| 1369278_at | guanine nucleotide binding protein, alpha 12 | Gna12 |
| 1368952_at | G protein-coupled receptor 51 | Gpr51 |
| 1388085_at | glutathione peroxidase 6 | Gpx6 |
| 1391731_at | high mobility group box transcription factor 1 | Hbp1 |
| 1382902_at | potential ubiquitin ligase | Herc6 |
| AFFX_Rat_Hexokinase_3_at | hexokinase 1 | Hk1 |
| AFFX_Rat_Hexokinase_5_at | hexokinase 1 | Hk1 |
| AFFX_Rat_Hexokinase_M_at | hexokinase 1 | Hk1 |
| 1388080_a_at | histamine receptor H3 | Hrh3 |
| 1382697_at | heterogeneous nuclear ribonucleoprotein methyltransferase-like 3 (S. cerevisiae) | Hrmt1l3 |
| 1387994_at | hydroxysteroid (17-beta) dehydrogenase 9 | Hsd17b9 |
| 1375335_at | heat shock 90 kDa protein 1, beta | Hspcb |
| 1369456_at | 5-hydroxytryptamine (serotonin) receptor 2B | Htr2b |
| 1371442_at | hypoxia up-regulated 1 | Hyou1 |
| 1368416_at | integrin binding sialoprotein | Ibsp |
| 1391489_at | interferon inducible protein 1 (predicted) | Ifi1_predicted |
| 1381014_at | interferon-induced protein 44 (predicted) | Ifi44_predicted |
| 1384180_at | interferon-induced protein with tetratricopeptide repeats 2 (predicted) | Ifit2_predicted |
| 1379568_at | interferon-induced protein with tetratricopeptide repeats 2 (predicted) | Ifit2_predicted |
| 1371148_s_at | internexin, alpha | Inexa |
| 1383564_at | interferon regulatory factor 7 (predicted) | Irf7_predicted |
| 1376845_at | putative ISG12(b) protein | isg12(b) |
| 1383448_at | interferon dependent positive acting transcription factor 3 gamma (predicted) | Isgf3g_predicted |
| 1374627_at | interferon dependent positive acting transcription factor 3 gamma (predicted) | Isgf3g_predicted |
| 1374404_at | v-jun sarcoma virus 17 oncogene homolog (avian) | Jun |
| 1396701_at | kalirin, RhoGEF kinase | Kalrn |
| 1382787_at | kinesin family member 5A | Kif5a |
| 1388125_a_at | kinesin 2 | Kns2 |
| 1383145_at | lysosomal-associated membrane protein 3 (predicted) | Lamp3_predicted |
| 1387946_at | lectin, galactoside-binding, soluble, 3 binding protein | Lgals3bp |
| 1370853_at | CaM-kinase II inhibitor alpha | LOC287005 |
| 1385546_at | similar to IQ motif and WD repeats 1 | LOC289181 |
| 1371382_at | similar to Filamin A (Alpha-filamin) (Filamin 1) (Endothelial actin-binding | LOC293860 |
| | protein) (Actin-binding protein 280) (ABP-280) (Nonmuscle filamin) | |
| 1397512_at | similar to Vps41 protein | LOC306991 |
| 1393436_at | similar to potential ligand-binding protein | LOC309100 |
| 1379748_at | similar to minor histocompatibility antigen precursor | LOC310968 |
| 1389034_at | similar to ubiquitin specific protease UBP43 | LOC312688 |
| 1396803_at | similar to THO complex 2 | LOC313308 |
| 1399073_at | similar to HSPC263 | LOC314660 |
| 1377498_at | similar to adenosine monophosphate deaminase 2 (isoform L) | LOC362015 |
| 1391564_at | similar to zinc finger protein 426 | LOC363022 |
| 1394299_at | similar to brain expressed X-linked protein 2 | LOC363498 |
| 1373975_at | similar to thioether S-methyltransferase | LOC368066 |
| 1381556_at | similar to BC013672 protein | LOC498673 |
| 1384547_at | similar to antimicrobial peptide RY2G5 | LOC499925 |
| 1376693_at | similar to OEF2 | LOC500011 |
| 1397815_at | similar to ADP-ribosylation factor-like 10C | LOC500282 |
| 1387373_at | myomegalin | LOC64183 |
| 1368666_a_at | calcium-independent alpha-latrotoxin receptor homolog 3 | Lphn3 |
| 1384361_at | mal, T-cell differentiation protein 2 | Mal2 |
| 1387737_at | methionine adenosyltransferase II, alpha | Mat2a |
| 1387341_a_at | myelin basic protein | Mbp |
| 1381403_at | malic enzyme 2, NAD(+)-dependent, mitochondrial (predicted) | Me2_predicted |
| 1372389_at | similar to immediate early response 2 | MGC72578 |
| 1379891_at | scotin | MGC94600 |
| 1370129_at | meningioma expressed antigen 5 (hyaluronidase) | Mgea5 |
| 1388204_at | matrix metallopeptidase 13 | Mmp13 |
| 1372800_at | myeloid ecotropic viral integration site-related gene 2 (predicted) | Mrg2_predicted |
| 1371015_at | myxovirus (influenza virus) resistance 1 | Mx1 |
| 1369202_at | myxovirus (influenza virus) resistance 2 | Mx2 |
| 1387283_at | myxovirus (influenza virus) resistance 2 | Mx2 |
| 1393881_at | NMDA receptor-regulated gene 1 (predicted) | Narg1_predicted |
| 1392581_at | neurocalcin delta | Ncald |
| 1375119_at | neural precursor cell expressed, developmentally down-regulated gene 4A | Nedd4a |
| 1368820_at | nuclear transcription factor-Y gamma | Nfyc |
| 1395408_at | nitric oxide synthase trafficking | Nostrin |
| 1386935_at | nuclear receptor subfamily 4, group A, member 1 | Nr4a1 |
| 1369689_at | N-ethylmaleimide sensitive fusion protein | Nsf |
| 1396684_at | NTE-related protein | Ntel |
| 1387639_at | neural visinin-like Ca2+-binding protein type 2 | Nvjp2 |
| 1369835_at | olfactory marker protein | Omp |
| 1390923_a_at | oxysterol binding protein-like 1A | Osbpl1a |
| 1369209_at | p34 protein | P34 |
| 1368958_at | protein kinase C and casein kinase substrate in neurons 1 | Pacsin1 |
| 1396267_at | p21 (CDKN1A)-activated kinase 2 | Pak2 |
| 1385116_at | protocadherin beta 21 (predicted) | Pcdhb21_predicted |
| 1377122_at | phosphodiesterase 1A, calmodulin-dependent | Pde1a |
| 1369044_a_at | phosphodiesterase 4B | Pde4b |
| 1370833_at | peroxin 2 | Pex2 |
| 1367970_at | profilin 2 | Pfn2 |
| 1387238_at | paired-like homeobox 2a | Phox2a |
| 1376137_at | pleckstrin homology domain containing, family B (evectins) member 2 | Plekhb2_predicted |
| | (predicted) | |
| 1368213_at | P450 (cytochrome) oxidoreductase | Por |
| 1395236_at | protein phosphatase 1, regulatory (inhibitor) subunit 3C (predicted) | Ppp1r3c_predicted |
| 1381024_at | protein phosphatase 2 (formerly 2A), regulatory subunit A (PR 65), alpha | Ppp2r1a |
| | isoform | |
| 1369297_at | protein phosphatase 2 (formerly 2A), regulatory subunit B (PR 52), gamma | Ppp2r2c |
| | isoform | |
| 1395409_at | protein phosphatase 2A, regulatory subunit B′ (PR 53) (predicted) | Ppp2r4_predicted |
| 1369152_at | protein phospatase 3, regulatory subunit B, alpha isoform, type 1 | Ppp3r1 |
| 1394416_at | prickle-like 2 (Drosophila) (predicted) | Prickle2_predicted |
| 1388182_at | DNA primase, p49 subunit | Prim1 |
| 1368240_a_at | protein kinase C, beta 1 | Prkcb1 |
| 1387242_at | Protein kinase, interferon-inducible double stranded RNA dependent | Prkr |
| 1375476_at | muscle glycogen phosphorylase | Pygm |
| 1376029_at | RAB2, member RAS oncogene family-like | Rab2l |
| 1393288_at | RAB5B, member RAS oncogene family (predicted) | Rab5b_predicted |
| 1371103_at | RAB6B, member RAS oncogene family | Rab6b |
| 1398838_at | RAB7, member RAS oncogene family | Rab7 |
| 1389811_at | RasGEF domain family, member 1C (predicted) | Rasgef1c_predicted |
| 1369129_at | RAS guanyl releasing protein 1 | Rasgrp1 |
| 1387622_at | radical fringe gene homolog (Drosophila) | Rfng |
| 1395739_at | similar to RIKEN cDNA C920006C10 (predicted) | RGD1305976_predicted |
| 1379285_at | similar to 5830458K16Rik protein (predicted) | RGD1306974_predicted |
| 1391908_at | similar to retinoid × receptor interacting protein (predicted) | RGD1307009_predicted |
| 1389044_at | similar to golgi-specific brefeldin A-resistance guanine nucleotide exchange | RGD1307160_predicted |
| | factor 1 (predicted) | |
| 1376144_at | similar to B aggressive lymphoma (predicted) | RGD1307534_predicted |
| 1377683_at | similar to hypothetical protein FLJ13045 (predicted) | RGD1307615_predicted |
| 1385105_at | similar to DKFZP434B168 protein (predicted) | RGD1308014_predicted |
| 1375829_at | similar to RIKEN cDNA 1810055E12 (predicted) | RGD1308326_predicted |
| 1395533_at | similar to KIAA0564 protein (predicted) | RGD1308772_predicted |
| 1377950_at | similar to interferon-inducible GTPase (predicted) | RGD1309362_predicted |
| 1396137_at | similar to RIKEN cDNA C230075L19 gene (predicted) | RGD1310284_predicted |
| 1372034_at | similar to hypothetical protein MGC29390 (predicted) | RGD1310490_predicted |
| 1384334_at | similar to RIKEN cDNA C330023F11 (predicted) | RGD1311440_predicted |
| 1374142_at | similar to RIKEN cDNA E130201N16 (predicted) | RGD1311589_predicted |
| 1394427_at | similar to 4921517L17Rik protein (predicted) | RGD1311678_predicted |
| 1398444_at | similar to RIKEN cDNA 5133400G04 (predicted) | RGD1311742_predicted |
| 1391249_at | similar to DD1 (predicted) | RGD1312005_predicted |
| 1368144_at | regulator of G-protein signaling 2 | Rgs2 |
| 1369332_a_at | regulating synaptic membrane exocytosis 1 | RIMS1 |
| 1395699_at | RIO kinase 3 (yeast) (predicted) | Riok3_predicted |
| 1398824_at | coated vesicle membrane protein | Rnp24 |
| 1375788_at | ribosomal protein L7 | Rpl7 |
| 1390777_at | sterol-C5-desaturase (fungal ERG3, delta-5-desaturase) homolog (S. cerevisae) | Sc5d |
| 1387926_at | sterol-C5-desaturase (fungal ERG3, delta-5-desaturase) homolog (S. cerevisae) | Sc5d |
| 1386889_at | stearoyl-Coenzyme A desaturase 2 | Scd2 |
| 1369265_at | SUMO/sentrin specific protease 2 | Senp2 |
| 1371310_s_at | serine (or cysteine) proteinase inhibitor, clade H, member 1 | Serpinh1 |
| 1371063_at | SH3 domain protein 2A | Sh3gl2 |
| 1368986_at | solute carrier family 17 (sodium-dependent inorganic phosphate cotransporter), | Slc17a7 |
| | member 7 | |
| 1388064_a_at | solute carrier family 1 (glial high affinity glutamate transporter), member 3 | Slc1a3 |
| 1367810_at | choline transporter | Slc6a8 |
| 1387094_at | solute carrier organic anion transporter family, member 1a4 | Slco1a4 |
| 1367563_at | secreted acidic cysteine rich glycoprotein | Sparc |
| 1386541_at | spermatogenesis associated 9 (predicted) | Spata9_predicted |
| 1368835_at | signal transducer and activator of transcription 1 | Stat1 |
| 1387354_at | signal transducer and activator of transcription 1 | Stat1 |
| 1372757_at | signal transducer and activator of transcription 1 | Stat1 |
| 1368844_at | stress 70 protein chaperone, microsome-associated, 60 kD human homolog | Stch |
| 1397148_at | stomatin (Epb7.2)-like 3 (predicted) | Stoml3_predicted |
| 1387359_at | syntaxin 1A (brain) | Stx1a |
| 1384716_at | synaptotagmin 1 | Syt1 |
| 1387662_at | synaptotagmin 4 | Syt4 |
| 1370323_at | thimet oligopeptidase 1 | Thop1 |
| 1371194_at | tumor necrosis factor alpha induced protein 6 | Tnfaip6 |
| 1378572_at | toll interacting protein (predicted) | Tollip_predicted |
| 1395794_at | tropomyosin 1, alpha | Tpm1 |
| 1385252_at | tripartite motif protein 34 (predicted) | Trim34_predicted |
| 1367598_at | transthyretin | Ttr |
| 1373037_at | ubiquitin-conjugating enzyme E2L 6 (predicted) | Ube2l6_predicted |
| 1384053_at | ubiquitin conjugation factor E4 A | Ube4a |
| 1368858_at | UDP-glucuronosyltransferase 8 | Ugt8 |
| 1388088_a_at | upstream transcription factor 2 | Usf2 |
| 1386909_a_at | voltage-dependent anion channel 1 | Vdac1 |
| 1396462_at | vacuolar protein sorting 4a (yeast) | Vps4a |
| 1396170_at | WW domain binding protein 4 (formin binding protein 21) | Wbp4 |
| 1376256_at | WD repeat and FYVE domain containing 1 (predicted) | Wdfy1_predicted |
| 1382192_at | extra cellular link domain-containing 1 (predicted) | Xlkd1_predicted |
| 1380071_at | zinc finger CCCH type domain containing 1 (predicted) | Zc3hdc1_predicted |
| 1394975_at | zinc finger, matrin-like (predicted) | Zfml_predicted |
| 1393080_at | zinc finger RNA binding protein (predicted) | Zfr_predicted |
| 1377837_at | zinc finger protein 183 (RING finger, C3HC4 type) | Znf183 |

Genes and gene fragments identified as changing significantly in animals that were reared in social isolation, relative to social control animals on postnatal day 60 (P60) are provided in Table 5.

TABLE 5

| Fragment Name | Gene Name | Gene Symbol |
|---|---|---|
| 1370116_at | septin 3 | 3-Sep |
| 1387436_at | septin 7 | 7-Sep |
| 1386906_a_at | septin 9 | 9-Sep |
| 1392108_at | (ATP-binding cassette, sub-family C (CFTR/MRP), member 3, aldehyde | (Abcc3, Aox3, Ccl9_predicted, |
| | oxidase 3, chemokine (C-C motif) ligand 9 (predicted), enoyl-Coenzyme A, | Ehhadh, Gmpr2, Tnfrsf1b) |
| | hydratase/3-hydroxyacyl Coenzyme A dehydrogenase, guanosine | |
| | monophosphate reductase 2, tumor necrosis factor recept | |
| 1383140_at | (5 nucleotidase, 6-pyruvoyl-tetrahydropterin synthase, Ras homolog enriched | (Agtrap, Apaf1, |
| | in brain like 1, angiotensin II receptor-associated protein, apoptotic peptidase | Cdkl1_predicted, Cln2, |
| | activating factor 1, ceroid-lipofuscinosis, neuronal 2, cyclin-dependent kinase- | Gcat_predicted, Hspa1b, Keg1, |
| | like 1 (CDC2-relat | LOC293989, MGC95001, Nt5, |
| | | Pgsg, Pts, Rhebl1, |
| | | Spock2_predicted, Tsarg1, |
| | | Ttc4_predicted, sag) |
| 1370686_at | (SEC6-like 1 (S. cerevisiae), aryl-hydrocarbon receptor repressor) | (Ahrr, Sec6l1) |
| 1383079_at | (apoptotic peptidase activating factor 1, ceroid-lipofuscinosis, neuronal 2, | (Apaf1, Ccr5, Cln2, Galm, |
| | chemokine (C-C) receptor 5, galactose mutarotase (aldose 1-epimerase), | Gcat_predicted, LOC501658, |
| | glycine C-acetyltransferase (2-amino-3-ketobutyrate-coenzyme A ligase) | Psmd9) |
| | (predicted), proteasome (prosome, ma | |
| 1383096_at | (LOC500962, amyloid beta (A4) precursor-like protein 2) | (Aplp2, LOC500962) |
| 1396944_at | (LOC500760, bromodomain adjacent to zinc finger domain, 2A (predicted)) | (Baz2a_predicted, LOC500760) |
| 1369211_at | (calcium channel, voltage-dependent, alpha 1I subunit, hypothetical gene | (Cacna1i, LOC497824) |
| | supported by NM_020084) | |
| 1382113_at | (chemokine (C-C motif) ligand 9 (predicted), similar to expressed sequence | (Ccl9_predicted, MGC72612) |
| | AI449175) | |
| 1372977_at | (atlastin-like, cyclin-dependent kinase-like 1 (CDC2-related kinase) | (Cdkl1_predicted, LOC362750, |
| | (predicted), similar to mitogen-activated protein kinase kinase kinase kinase | LOC503027) |
| | 5 isoform 2) | |
| 1389625_at | (coiled-coil-helix-coiled-coil-helix domain containing 4 (predicted), similar to | (Chchd4_predicted, |
| | Coiled-coil-helix-coiled-coil-helix domain containing 4) | LOC309309) |
| 1377671_at | (colony stimulating factor 2 receptor, beta 1, low-affinity (granulocyte- | (Csf2rb1, LOC497822) |
| | macrophage), hypothetical gene supported by NM_133555) | |
| 1368738_at | (cytochrome P450, family 11, subfamily B, polypeptide 2, cytochrome P450, | (Cyp11b1, Cyp11b2, |
| | subfamily 11B, polypeptide 1, similar to Cytochrome P450 11B1, | LOC500892) |
| | mitochondrial precursor (CYPXIB1) (P450C11) (Steroid 11-beta-hydroxylase) | |
| | (P450(11 beta)-DS)) | |
| 1372247_at | (dolichyl-di-phosphooligosaccharide-protein glycotransferase, dolichyl-di- | (Ddost, Ddost_predicted) |
| | phosphooligosaccharide-protein glycotransferase (predicted)) | |
| 1388057_a_at | (PSD-95 binding protein, discs, large (Drosophila) homolog-associated | (Dlgap1, LOC245710) |
| | protein 1) | |
| 1375388_at | (elongation factor RNA polymerase II-like 3 (predicted), glucose regulated | (EII3_predicted, Grp58) |
| | protein, 58 kDa) | |
| 1369621_s_at | (FK506 binding protein 2, FK506-binding protein 1a) | (Fkbp1a, Fkbp2) |
| AFFX_Rat_GAPDH_5_at | (glyceraldehyde-3-phosphate dehydrogenase, similar to glyceraldehyde-3- | (Gapd, LOC498123, |
| | phosphate dehydrogenase) | LOC500506) |
| 1395412_at | (GPI-anchored membrane protein 1 (predicted), similar to GPI-anchored | (Gpiap1_predicted, |
| | membrane protein 1) | LOC362174) |
| 1395173_at | (GPI-anchored membrane protein 1 (predicted), similar to GPI-anchored | (Gpiap1_predicted, |
| | membrane protein 1) | LOC362174) |
| 1370454_at | (HS1 binding protein, homer homolog 1 (Drosophila)) | (Homer1, Hs1bp1) |
| 1370997_at | (HS1 binding protein, homer homolog 1 (Drosophila)) | (Homer1, Hs1bp1) |
| 1369868_at | (hypothetical gene supported by NM_053946, implantation-associated | (lag2, LOC497846) |
| | protein) | |
| 1369323_at | (leptin receptor, leptin receptor overlapping transcript) | (Lepr, Leprot) |
| 1398875_at | (estrous-specific protein, 250 kDa, similar to RIKEN cDNA 1500004O14) | (LOC246270, LOC366277) |
| 1398303_s_at | (tropomyosin 3, gamma, tropomyosin isoform 6) | (LOC286890, Tpm3) |
| 1387617_at | (tropomyosin 3, gamma, tropomyosin isoform 6) | (LOC286890, Tpm3) |
| 1367452_at | (SMT3 suppressor of mif two 3 homolog 2 (yeast), similar to SMT3 | (LOC287993, Sumo2) |
| | suppressor of mif two 3 homolog 2) | |
| 1394943_at | (similar to RIKEN cDNA 1810020E01 (predicted), similar to tyrosine kinase- | (LOC293112, |
| | associated leucine zipper protein LAZipII) | RGD1305677_predicted) |
| 1383116_at | (similar to RIKEN cDNA 4833435D08, similar to glycosyltransferase 28 | (LOC300284, LOC367759) |
| | domain containing 1) | |
| 1393152_at | similar to Mut protein | (LOC301276, LOC363201) |
| 1391020_at | (similar to CG1841-PA, isoform A, similar to seven transmembrane helix | (LOC303660, LOC498020) |
| | receptor) | |
| 1387458_at | (ring finger protein 4, similar to CG14998-PC, isoform C) | (LOC305453, Rnf4) |
| 1397824_at | similar to WAC | (LOC307029, LOC361260) |
| 1398965_at | (eomesodermin, similar to golgi autoantigen golgin subtype a4; tGolgin-1) | (LOC316052, LOC501069) |
| 1382331_at | (similar to RIKEN cDNA 0610038L10 gene, similar to riboflavin kinase) | (LOC317214, LOC499328) |
| 1370500_a_at | (hypothetical gene supported by NM_012720, myelin-associated | (LOC360443, Mobp) |
| | oligodendrocytic basic protein) | |
| 1377174_at | (SET binding factor 1 (predicted), similar to mKIAA3020 protein) | (LOC362985, Sbf1_predicted) |
| 1372968_at | (SET binding factor 1 (predicted), similar to mKIAA3020 protein) | (LOC362985, Sbf1_predicted) |
| 1397522_at | (SET binding factor 1 (predicted), similar to mKIAA3020 protein) | (LOC362985, Sbf1_predicted) |
| 1390576_at | similar to RIKEN cDNA 4921537P18 | (LOC364620, LOC498649) |
| 1395363_at | (methionine-tRNA synthetase (predicted), similar to glyceraldehyde-3- | (LOC366910, Mars_predicted) |
| | phosphate dehydrogenase) | |
| 1382522_at | (hypothetical gene supported by NM_019149, matrin 3) | (LOC497720, Matr3) |
| 1367823_at | (similar to testis specific protein, Ddc8, tissue inhibitor of metalloproteinase 2) | (LOC498028, Timp2) |
| 1395426_at | similar to ubiquitin-conjugating enzyme E2 variant 1 | (LOC498326, LOC499945) |
| 1377986_at | (short coiled-coil protein (predicted), similar to short coiled-coil protein) | (LOC498931, Scoc_predicted) |
| 1384257_at | (non-POU domain containing, octamer-binding, similar to Non-POU-domain- | (LOC499170, LOC501589, |
| | containing, octamer binding protein, similar to Nono protein) | Nono) |
| 1384965_at | (LOC499369, serine/threonine kinase 2) | (LOC499369, Slk) |
| 1387971_a_at | (mitogen activated protein kinase 8 interacting protein, similar to RIKEN | (LOC499836, Mapk8ip) |
| | cDNA 1700029I15) | |
| 1390906_at | (similar to hypothetical protein 3010020C06, tropomodulin 1) | (LOC500455, Tmod1) |
| 1370081_a_at | (LOC501102, vascular endothelial growth factor A) | (LOC501102, Vegfa) |
| 1374129_at | (similar to RNA binding protein with multiple splicing 2, similar to product is | (LOC501454, LOC503214) |
| | unknown~seizure-related gene) | |
| 1383276_at | (LanC (bacterial lantibiotic synthetase component C)-like 2 (predicted), | (LOC503420, Lancl2_predicted) |
| | similar to LanC-like protein 2 (Testis-specific adriamycin sensitivity protein)) | |
| 1374606_at | (MYST histone acetyltransferase 1 (predicted), protease, serine, 8 | (Myst1_predicted, Prss8) |
| | (prostasin)) | |
| 1397468_at | (ROD1 regulator of differentiation 1 (S. pombe), similar to RIKEN cDNA | (RGD1305387_predicted, Rod1) |
| | 2610207I16 (predicted)) | |
| 1371839_at | (similar to hypothetical protein ET (predicted), similar to splicing factor, | (RGD1307394_predicted, Sfrs2) |
| | arginine/serine-rich 2) | |
| 1370428_x_at | (RT1 class I, A3, RT1 class I, CE10, RT1 class I, CE3, RT1 class I, CE7, RT1 | (RT1-A2, RT1-A3, RT1-Aw2, |
| | class Ia, locus A2, RT1 class Ib, locus Aw2) | RT1-CE10, RT1-CE3, RT1- |
| | | CE7) |
| 1388071_x_at | (RT1 class I, A3, RT1 class I, CE10, RT1 class I, CE7, RT1 class Ib, locus | (RT1-A3, RT1-Aw2, RT1-CE10, |
| | Aw2) | RT1-CE7) |
| 1388203_x_at | (RT1 class I, A3, RT1 class I, CE1, RT1 class I, CE3, RT1 class I, CE5) | (RT1-A3, RT1-CE1, RT1-CE3, |
| | | RT1-CE5) |
| 1369085_s_at | (SNRPN upstream reading frame, small nuclear ribonucleoprotein N) | (Snrpn, Snurf) |
| 1374780_at | (zinc finger protein 22 (KOX 15), zinc finger protein 422 (predicted)) | (Zfp422_predicted, Znf22) |
| 1395346_at | angio-associated migratory protein (predicted) | Aamp_predicted |
| 1368484_at | ATP-binding cassette, sub-family B (MDR/TAP), member 9 | Abcb9 |
| 1375468_at | ATP-binding cassette, sub-family C (CFTR/MRP), member 5 | Abcc5 |
| 1368465_at | amiloride-sensitive cation channel 1, neuronal (degenerin) | Accn1 |
| 1371140_a_at | amiloride-sensitive cation channel 1, neuronal (degenerin) | Accn1 |
| 1398294_at | actinin, alpha 1 | Actn1 |
| 1379687_at | ARP2 actin-related protein 2 homolog (yeast) (predicted) | Actr2_predicted |
| 1388161_at | a disintegrin and metalloprotease domain 10 | Adam10 |
| 1368973_at | adenosine deaminase, RNA-specific | Adar |
| 1368729_a_at | adenylate cyclase activating polypeptide 1 receptor 1 | Adcyap1r1 |
| 1370121_at | adducin 1 (alpha) | Add1 |
| 1397519_at | adiponectin receptor 2 (predicted) | Adipor2_predicted |
| 1369629_at | adenosine kinase | Adk |
| 1369402_at | activity-dependent neuroprotective protein | Adnp |
| 1370584_a_at | adenosine A1 receptor | Adora1 |
| 1388757_at | adrenergic receptor kinase, beta 1 | Adrbk1 |
| 1387861_at | amino-terminal enhancer of split | Aes |
| 1392763_at | 1-acylglycerol-3-phosphate O-acyltransferase 1 | Agpat1 |
| 1371748_at | 1-acylglycerol-3-phosphate O-acyltransferase 1 | Agpat1 |
| 1383759_at | ATP/GTP binding protein 1 (predicted) | Agtpbp1_predicted |
| 1391891_at | ATP/GTP binding protein 1 (predicted) | Agtpbp1_predicted |
| 1388922_at | aryl-hydrocarbon receptor-interacting protein | Aip |
| 1369069_at | A kinase (PRKA) anchor protein 1 | Akap1 |
| 1387493_at | A kinase (PRKA) anchor protein 5 | Akap5 |
| 1392079_at | A kinase (PRKA) anchor protein 7 | Akap7 |
| 1368862_at | v-akt murine thymoma viral oncogene homolog 1 | Akt1 |
| 1368832_at | murine thymoma viral (v-akt) oncogene homolog 2 | Akt2 |
| 1380381_at | asparagine-linked glycosylation 2 homolog (yeast, alpha-1,3- | Alg2_predicted |
| | mannosyltransferase) (predicted) | |
| 1371132_a_at | ankyrin 3, epithelial isoform g | Ank3 |
| 1396195_at | ankyrin repeat domain 13 (predicted) | Ankrd13_predicted |
| 1368908_at | annexin A4 | Anxa4 |
| 1377769_at | adaptor protein complex AP-1, sigma 1 (predicted) | Ap1s1_predicted |
| 1370662_a_at | adaptor-related protein complex 2, beta 1 subunit | Ap2b1 |
| 1386877_at | adaptor-related protein complex 2, sigma 1 subunit | Ap2s1 |
| 1387289_at | amyloid beta (A4) precursor protein-binding, family A, member 2 | Apba2 |
| 1397670_at | autophagy 12-like (S. cerevisiae) (predicted) | Apg12l_predicted |
| 1382735_at | autophagy 12-like (S. cerevisiae) (predicted) | Apg12l_predicted |
| 1368258_at | apelin, AGTRL1 ligand | Apln |
| 1380533_at | amyloid beta (A4) precursor protein | App |
| 1387068_at | activity regulated cytoskeletal-associated protein | Arc |
| 1391658_at | archain | Arcn1 |
| 1398817_at | ADP-ribosylation factor 1 | Arf1 |
| 1387447_at | ADP-ribosylation factor 3 | Arf3 |
| 1384938_at | Rho GTPase activating protein 1 (predicted) | Arhgap1_predicted |
| 1383813_at | ADP-ribosylation factor-like 2 binding protein | Arl2bp |
| 1369729_at | ADP-ribosylation factor-like 5 | Arl5 |
| 1384131_at | ADP-ribosylation factor-like 6 interacting protein 2 (predicted) | Arl6ip2_predicted |
| 1369319_at | ADP-ribosylation factor-like 6 interacting protein 5 | Arl6ip5 |
| 1370611_at | aryl hydrocarbon receptor nuclear translocator 2 | Arnt2 |
| 1375137_at | actin related protein 2/3 complex, subunit 2 (predicted) | Arpc2_predicted |
| 1394318_at | cAMP-regulated phosphoprotein 19 | Arpp19 |
| 1369077_at | N-acylsphingosine amidohydrolase 1 | Asah1 |
| 1383535_at | ankyrin repeat and SOCS box-containing protein 8 (predicted) | Asb8_predicted |
| 1370964_at | arginosuccinate synthetase | Ass |
| 1371108_a_at | ATPase, Na+/K+ transporting, alpha 1 polypeptide | Atp1a1 |
| 1368701_at | ATPase, Na+/K+ transporting, alpha 3 polypeptide | Atp1a3 |
| 1369798_at | ATPase, Na+/K+ transporting, beta 2 polypeptide | Atp1b2 |
| 1387285_at | ATPase, Ca++ transporting, plasma membrane 2 | Atp2b2 |
| 1396279_at | ATPase, H+ transporting, lysosomal accessory protein 2 | Atp6ap2 |
| 1387664_at | ATPase, H+ transporting, V1 subunit B, isoform 2 | Atp6v1b2 |
| 1369256_at | beta-site APP cleaving enzyme | Bace |
| 1374117_at | brain-specific angiogenesis inhibitor 1-associated protein 2 | Baiap2 |
| 1369520_a_at | branched chain aminotransferase 1, cytosolic | Bcat1 |
| 1368999_a_at | brain-enriched guanylate kinase-associated | Begain |
| 1388733_at | bifunctional apoptosis regulator (predicted) | Bfar_predicted |
| 1369032_at | bladder cancer associated protein homolog (human) | Blcap |
| 1393641_at | B-cell linker | Blnk |
| 1398004_at | BMP/retinoic acid-inducible neural-specific protein 2 | Brinp2 |
| 1386774_at | BMP/retinoic acid-inducible neural-specific protein 2 | Brinp2 |
| 1387629_at | B-box and SPRY domain containing | Bspry |
| 1369886_a_at | calcium binding protein 1 | Cabp1 |
| 1370757_at | calcium channel, voltage-dependent, gamma subunit 3 | Cacng3 |
| 1370775_a_at | calcitonin/calcitonin-related polypeptide, alpha | Calca |
| 1367889_at | calcium/calmodulin-dependent protein kinase I | Camk1 |
| 1377518_at | calcium/calmodulin-dependent protein kinase I gamma | Camk1g |
| 1371263_a_at | calcium/calmodulin-dependent protein kinase II, delta | Camk2d |
| 1369993_at | calcium/calmodulin-dependent protein kinase II gamma | Camk2g |
| 1371687_at | calnexin | Canx |
| 1368808_at | CAP, adenylate cyclase-associated protein 1 (yeast) | Cap1 |
| 1368894_at | CAP, adenylate cyclase-associated protein, 2 (yeast) | Cap2 |
| 1387292_s_at | calpain 8 | Capn8 |
| 1391572_at | cysteinyl-tRNA synthetase (predicted) | Cars_predicted |
| 1397571_at | metastatic lymph node 51 | Casc3 |
| 1387401_at | calsequestrin 2 | Casq2 |
| 1369733_at | catenin beta | Catnb |
| 1382842_at | cyclin H | Ccnh |
| 1395508_at | chaperonin containing TCP1, subunit 5 (epsilon) | Cct5 |
| 1394997_at | congenital dyserythropoietic anemia, type I (predicted) | Cdan1_predicted |
| 1371278_at | cell division cycle 34 homolog (S. cerevisiae) (predicted) | Cdc34_predicted |
| 1387695_at | Cdc42-binding protein kinase beta | Cdc42bpb |
| 1375719_s_at | cadherin 13 | Cdh13 |
| 1395122_s_at | cyclin-dependent kinase (CDC2-like) 10 (predicted) | Cdk10_predicted |
| 1394320_at | cyclin dependent kinase 2 | Cdk2 |
| 1369538_at | cyclin-dependent kinase 5, regulatory subunit 1 (p35) | Cdk5r |
| 1393441_at | similar to cyclin-dependent kinase inhibitor 2D | Cdkn2d |
| 1387636_a_at | P11 protein | Cdtw1 |
| 1379130_at | CDW92 antigen | Cdw92 |
| 1387726_at | caudal type homeo box 2 | Cdx2 |
| 1368449_at | centaurin, alpha 1 | Centa1 |
| 1370184_at | cofilin 1 | Cfl1 |
| 1397200_at | chromodomain helicase DNA binding protein 4 | Chd4 |
| 1387388_at | calcium binding protein p22 | Chp |
| 1369112_at | cholinergic receptor, muscarinic 3 | Chrm3 |
| 1369019_at | cholinergic receptor, nicotinic, alpha polypeptide 5 | Chrna5 |
| 1367740_at | creatine kinase, brain | Ckb |
| 1384015_at | cytoplasmic linker associated protein 1 (predicted) | Clasp1_predicted |
| 1369791_at | putative chloride channel (similar to Mm Clcn4-2) | Clcn4-2 |
| 1369609_at | claudin 11 | Cldn11 |
| 1377765_at | chloride intracellular channel 4 | Clic4 |
| 1384336_at | Cln3p | Cln3 |
| 1370991_at | camello-like 3 | Cml3 |
| 1388176_at | camello-like 5 | Cml5 |
| 1370693_a_at | cyclic nucleotide phosphodiesterase 1 | Cnp1 |
| 1394008_x_at | ciliary neurotrophic factor receptor | Cntfr |
| 1376868_at | Cobl-like 1 (predicted) | Cobll1_predicted |
| 1395352_at | COP9 constitutive photomorphogenic homolog subunit 3 | Cops3 |
| 1395565_at | COP9 signalosome subunit 4 | Cops4 |
| 1399010_at | COP9 (constitutive photomorphogenic) homolog, subunit 7a (Arabidopsis | Cops7a_predicted |
| | thaliana) (predicted) | |
| 1389897_at | COP9 (constitutive photomorphogenic) homolog, subunit 7a (Arabidopsis | Cops7a_predicted |
| | thaliana) (predicted) | |
| 1394565_at | coatomer protein complex, subunit zeta 1 (predicted) | Copz1_predicted |
| 1369811_at | complexin 1 | Cplx1 |
| 1377549_at | copine V (predicted) | Cpne5_predicted |
| 1373429_at | cAMP responsive element binding protein-like 1 | Crebl1 |
| 1378925_at | cAMP responsive element modulator | Crem |
| 1382037_at | cysteine-rich motor neuron 1 (predicted) | Crim1_predicted |
| 1387183_at | carnitine O-octanoyltransferase | Crot |
| 1376051_at | crystallin, lamda 1 | Cryl1 |
| 1384201_at | citrate synthase | Cs |
| 1369967_at | citrate synthase | Cs |
| 1395914_at | casein kinase 1, delta | Csnk1d |
| 1370527_a_at | casein kinase 1, delta | Csnk1d |
| 1386693_at | casein kinase 1, epsilon | Csnk1e |
| 1394731_at | casein kinase 1, gamma 3 | Csnk1g3 |
| 1368685_at | chondroitin sulfate proteoglycan 4 | Cspg4 |
| 1368704_a_at | chondroitin sulfate proteoglycan 5 | Cspg5 |
| 1379238_at | CTD (carboxy-terminal domain, RNA polymerase II, polypeptide A) small | Ctdspl_predicted |
| | phosphatase-like (predicted) | |
| 1397808_at | cathepsin C | Ctsc |
| 1367651_at | cathepsin D | Ctsd |
| 1370922_at | cortexin | Ctxn |
| 1387879_a_at | CUG triplet repeat, RNA-binding protein 2 | Cugbp2 |
| 1387880_at | CUG triplet repeat, RNA-binding protein 2 | Cugbp2 |
| 1379365_at | chemokine (C—X—C motif) ligand 11 | Cxcl11 |
| 1379911_at | death associated protein kinase 1 (predicted) | Dapk1_predicted |
| 1387874_at | D site albumin promoter binding protein | Dbp |
| 1389553_at | dendritic cell inhibitory receptor 3 | Dcir3 |
| 1371834_at | dynactin 2 | Dctn2 |
| 1399162_a_at | damage-specific DNA binding protein 1 | Ddb1 |
| 1397405_at | DEAD (Asp-Glu-Ala-Asp) box polypeptide 17 (predicted) | Ddx17_predicted |
| 1385214_at | DEAD (Asp-Glu-Ala-Asp) box polypeptide 3, X-linked | Ddx3x |
| 1381384_at | degenerative spermatocyte homolog (Drosophila) | Degs |
| 1385871_at | DEAH (Asp-Glu-Ala-His) box polypeptide 36 (predicted) | Dhx36_predicted |
| 1380807_at | discs, large homolog 1 (Drosophila) | Dlgh1 |
| 1368944_at | discs, large homolog 1 (Drosophila) | Dlgh1 |
| 1388280_a_at | discs, large homolog 3 (Drosophila) | Dlgh3 |
| 1382024_at | DnaJ (Hsp40) homolog, subfamily B, member 6 (predicted) | Dnajb6_predicted |
| 1369714_at | DnaJ (Hsp40) homolog, subfamily C, member 14 | Dnajc14 |
| 1369390_a_at | dipeptidylpeptidase 6 | Dpp6 |
| 1391602_at | developmentally regulated GTP binding protein 1 (predicted) | Drg1_predicted |
| 1368146_at | dual specificity phosphatase 1 | Dusp1 |
| 1377064_at | dual specificity phosphatase 6 | Dusp6 |
| 1391510_at | estrogen receptor-binding fragment-associated gene 9 (predicted) | Ebag9_predicted |
| 1395586_at | eukaryotic translation elongation factor 1 alpha 1 | Eef1a1 |
| 1385625_at | EGF-containing fibulin-like extracellular matrix protein 2 | Efemp2 |
| 1387306_a_at | early growth response 2 | Egr2 |
| 1387442_at | early growth response 4 | Egr4 |
| 1372317_at | EH-domain containing 1 (predicted) | Ehd1_predicted |
| 1387588_at | EH-domain containing 3 | Ehd3 |
| 1368866_at | GERp95 | Eif2c2 |
| 1397693_at | eukaryotic translation initiation factor 2, subunit 3, structural gene X-linked | Eif2s3x |
| 1387630_at | ELOVL family member 5, elongation of long chain fatty acids (yeast) | Elovl5 |
| 1390174_at | echinoderm microtubule associated protein like 1 (predicted) | Eml1_predicted |
| 1369702_at | endosulfine alpha | Ensa |
| 1369212_s_at | erythrocyte protein band 4.1-like 1 | Epb4.1l1 |
| 1388061_a_at | Eph receptor A7 | Epha7 |
| 1369453_at | Epsin 1 | Epn1 |
| 1387849_at | epsin 2 | Epn2 |
| 1370007_at | protein disulfide isomerase related protein (calcium-binding protein, intestinal- | Erp70 |
| | related) | |
| 1370625_at | Fas apoptotic inhibitory molecule 2 | Faim2 |
| 1392339_at | phenylalanine-tRNA synthetase-like, beta subunit | Farslb |
| 1392875_at | F-box and leucine-rich repeat protein 4 (predicted) | Fbxl4_predicted |
| 1376500_at | F-box only protein 23 (predicted) | Fbxo23_predicted |
| 1381903_at | F-box only protein 33 (predicted) | Fbxo33_predicted |
| 1370707_at | FEV (ETS oncogene family) | Fev |
| 1387498_a_at | Fibroblast growth factor receptor 1 | Fgfr1 |
| 1384829_at | fibroblast growth factor receptor 3 | Fgfr3 |
| 1390049_at | four and a half LIM domains 1 | Fhl1 |
| 1390995_at | FK506 binding protein-like | Fkbpl |
| 1369087_at | FMS-like tyrosine kinase 1 | Flt1 |
| 1375043_at | FBJ murine osteosarcoma viral oncogene homolog | Fos |
| 1395802_at | fucosyltransferase 11 | Fut11 |
| 1373435_at | fragile X mental retardation gene 2, autosomal homolog (predicted) | Fxr2h_predicted |
| 1368696_at | FXYD domain-containing ion transport regulator 7 | Fxyd7 |
| 1369103_at | fyn proto-oncogene | Fyn |
| 1381189_at | fizzy/cell division cycle 20 related 1 (Drosophila) (predicted) | Fzr1_predicted |
| 1378239_at | glucosidase, alpha; acid (Pompe disease, glycogen storage disease type II) | Gaa |
| 1388030_a_at | gamma-aminobutyric acid (GABA) B receptor 1 | Gabbr1 |
| 1369371_a_at | gamma-aminobutyric acid (GABA) B receptor 1 | Gabbr1 |
| 1369905_at | gamma-aminobutyric acid (GABA-A) receptor, subunit alpha 4 | Gabra4 |
| 1395269_s_at | gamma-aminobutyric acid A receptor, delta | Gabrd |
| 1370760_a_at | glutamate decarboxylase 1 | Gad1 |
| 1372016_at | growth arrest and DNA-damage-inducible 45 beta (predicted) | Gadd45b_predicted |
| 1368332_at | guanylate nucleotide binding protein 2 | Gbp2 |
| 1375135_at | GCN1 general control of amino-acid synthesis 1-like 1 (yeast) (predicted) | Gcn1l1_predicted |
| 1398822_at | GDP dissociation inhibitor 2 | Gdi2 |
| 1375359_at | glial cell line derived neurotrophic factor family receptor alpha 2 | Gfra2 |
| 1373803_a_at | growth hormone receptor | Ghr |
| 1370730_a_at | growth hormone releasing hormone receptor | Ghrhr |
| 1368775_at | gonadotropin inducible ovarian transcription factor 1 | Giot1 |
| 1369640_at | gap junction membrane channel protein alpha 1 | Gja1 |
| 1370146_at | glycine receptor, beta subunit | Glrb |
| 1367705_at | glutaredoxin 1 (thioltransferase) | Glrx1 |
| 1367633_at | glutamine synthetase 1 | Glul |
| 1387505_at | guanine nucleotide binding protein, alpha inhibiting 1 | Gnai1 |
| 1367844_at | guanine nucleotide binding protein, alpha inhibiting 2 | Gnai2 |
| 1368030_at | guanine nucleotide binding protein, alpha inhibiting 3 | Gnai3 |
| 1381988_at | guanine nucleotide binding protein, alpha q polypeptide | Gnaq |
| 1387095_at | guanine nucleotide binding protein, alpha z subunit | Gnaz |
| 1368957_at | guanine nucleotide binding protein, gamma 7 | Gng7 |
| 1369571_at | golgi phosphoprotein 3 | Golph3 |
| 1369410_at | golgi SNAP receptor complex member 1 | Gosr1 |
| 1368272_at | glutamate oxaloacetate transaminase 1 | Got1 |
| 1382739_at | G protein-coupled receptor 19 | Gpr19 |
| 1368952_at | G protein-coupled receptor 51 | Gpr51 |
| 1388066_a_at | G protein-coupled receptor kinase 6 | Gprk6 |
| 1370590_at | G-protein signalling modulator 1 (AGS3-like, C. elegans) | Gpsm1 |
| 1386871_at | glutathione peroxidase 4 | Gpx4 |
| 1389900_at | glutamate receptor, ionotropic, N-methyl D-aspartate 1 | Grin1 |
| 1371051_at | glutamate receptor, ionotropic, N-methyl D-aspartate-like 1A | Grinl1a |
| 1369781_at | glutamate receptor, metabotropic 7 | Grm7 |
| 1383627_a_at | general transcription factor IIF, polypeptide 1, 74 kDa | Gtf2f1 |
| 1387390_at | granzyme K | Gzmk |
| 1367759_at | H1 histone family, member 0 | H1f0 |
| 1398849_at | H3 histone, family 3B | H3f3b |
| AFFX_Rat_Hexokinase_3_at | hexokinase 1 | Hk1 |
| AFFX_Rat_Hexokinase_5_at | hexokinase 1 | Hk1 |
| AFFX_Rat_Hexokinase_M_at | hexokinase 1 | Hk1 |
| 1386983_at | hydroxymethylbilane synthase | Hmbs |
| 1388309_at | high mobility group AT-hook 1 | Hmga1 |
| 1387848_at | 3-hydroxy-3-methylglutaryl-Coenzyme A reductase | Hmgcr |
| 1375612_at | heterogeneous nuclear ribonucleoprotein A1 | Hnrpa1 |
| 1369562_at | neural visinin-like Ca2+-binding protein type 3 | Hpcal1 |
| 1371510_at | HCF-1 beta-propeller interacting protein | Hpip |
| 1393790_at | HRAS-like suppressor (predicted) | Hrasls_predicted |
| 1388080_a_at | histamine receptor H3 | Hrh3 |
| 1391423_at | heat shock factor 2 | Hsf2 |
| 1387430_at | heat shock factor 2 | Hsf2 |
| 1385620_at | heat shock protein 105 (predicted) | Hsp105_predicted |
| 1369124_at | 5-hydroxytryptamine (serotonin) receptor 2A | Htr2a |
| 1371442_at | hypoxia up-regulated 1 | Hyou1 |
| 1393710_at | intercellular adhesion molecule 5, telencephalin (predicted) | Icam5_predicted |
| 1387769_a_at | Inhibitor of DNA binding 3, dominant negative helix-loop-helix protein | Id3 |
| 1385923_at | inhibitor of DNA binding 4 | Idb4 |
| 1375183_at | inhibitor of DNA binding 4 | Idb4 |
| 1375696_at | interferon (alpha and beta) receptor 1 (predicted) | Ifnar1_predicted |
| 1394668_at | immunoglobulin superfamily, member 4B (predicted) | Igsf4b_predicted |
| 1390033_at | immunoglobulin superfamily, member 4B (predicted) | Igsf4b_predicted |
| 1395339_at | imprinted and ancient | Impact |
| 1375754_at | imprinted and ancient | Impact |
| 1370232_at | isovaleryl coenzyme A dehydrogenase | Ivd |
| 1368251_at | Janus kinase 3 | Jak3 |
| 1369847_at | potassium voltage-gated channel, shaker-related subfamily, beta member 1 | Kcnab1 |
| 1370558_a_at | potassium voltage gated channel, Shaw-related subfamily, member 2 | Kcnc2 |
| 1370773_a_at | Kv channel-interacting protein 2 | Kcnip2 |
| 1370595_a_at | potassium channel interacting protein 4 | Kcnip4 |
| 1368793_at | potassium inwardly-rectifying channel, subfamily J, member 2 | Kcnj2 |
| 1369487_a_at | potassium inwardly-rectifying channel, subfamily J, member 4 | Kcnj4 |
| 1369418_at | potassium inwardly-rectifying channel, subfamily J, member 9 | Kcnj9 |
| 1375190_at | potassium channel tetramerisation domain containing 13 | Kctd13 |
| 1387260_at | Kruppel-like factor 4 (gut) | Klf4 |
| 1385961_at | Kruppel-like factor 5 | Klf5 |
| 1397386_at | lymphoid nuclear protein related to AF4-like (predicted) | Laf4l_predicted |
| 1387844_at | LIM and SH3 protein 1 | Lasp1 |
| 1388747_at | leucine carboxyl methyltransferase 1 | Lcmt1 |
| 1381369_at | leucine-rich repeat LGI family, member 3 (predicted) | Lgi3_predicted |
| 1370108_a_at | lin-7 homolog a (C. elegans) | Lin7a |
| 1369610_at | lin-7 homolog C (C. elegans) | Lin7c |
| 1390379_at | LIM domain only 4 (predicted) | Lmo4_predicted |
| 1379803_at | LIM domain only 4 (predicted) | Lmo4_predicted |
| 1369408_at | BMP/retinoic acid-inducible neural-specific protein | LOC140610 |
| 1384608_at | RN protein | LOC171116 |
| 1369922_at | RDCR-0918-3 protein | LOC246120 |
| 1397843_at | WD-containing protein Rab11BP/Rabphilin-11 | LOC246152 |
| 1397846_at | cytosolic leucine-rich protein | LOC246768 |
| 1370853_at | CaM-kinase II inhibitor alpha | LOC287005 |
| 1398316_at | similar to LEYDIG CELL TUMOR 10 KD PROTEIN | LOC288913 |
| 1392723_at | similar to ankyrin repeat domain protein 17 isoform b | LOC289521 |
| 1394536_at | similar to Ubiquitin carboxyl-terminal hydrolase 4 (Ubiquitin thiolesterase 4) | LOC290864 |
| | (Ubiquitin-specific processing protease 4) (Deubiquitinating enzyme 4) | |
| | (Ubiquitous nuclear protein) | |
| 1371365_at | similar to Ubiquitin-conjugating enzyme E2S | LOC292588 |
| 1373149_at | similar to yippee-like 3 | LOC293491 |
| 1399155_at | similar to RIKEN cDNA 5830434P21 | LOC296637 |
| 1388315_at | similar to neuronal protein 15.6 | LOC299310 |
| 1378149_at | similar to RIKEN cDNA 2310075A12 | LOC300472 |
| 1392122_x_at | similar to Btk-PH-domain binding protein | LOC301119 |
| 1395379_at | similar to U2af1-rs2 | LOC302670 |
| 1383335_at | hypothetical protein LOC303515 | LOC303515 |
| 1374579_at | similar to RIKEN cDNA E230015L20 gene | LOC306238 |
| 1397512_at | similar to Vps41 protein | LOC306991 |
| 1396106_at | similar to dystrobrevin alpha isoform 1 | LOC307548 |
| 1391786_at | similar to Murine homolog of human ftp-3 | LOC308650 |
| 1379123_at | similar to Seizure related 6 homolog (mouse)-like 2 | LOC308988 |
| 1390175_at | similar to Delta-interacting protein A (Hepatitis delta antigen interacting | LOC309161 |
| | protein A) | |
| 1375311_at | similar to Delta-interacting protein A (Hepatitis delta antigen interacting | LOC309161 |
| | protein A) | |
| 1384525_at | similar to spleen specific 238 kDa protein with PH domain | LOC313438 |
| 1380314_at | similar to hypothetical protein FLJ38984 | LOC313595 |
| 1373018_at | similar to hypothetical protein FLJ14800 | LOC315327 |
| 1384550_at | similar to RIKEN cDNA 1810030O07 | LOC317344 |
| 1373603_at | similar to RIKEN cDNA 0610007P22 | LOC360494 |
| 1382805_at | similar to myosin XVIIIa | LOC360570 |
| 1383555_at | similar to RIKEN cDNA 4930579G22 | LOC360799 |
| 1393604_at | similar to RIKEN cDNA A930018B01 | LOC360810 |
| 1381611_at | similar to transcription factor ELYS | LOC360886 |
| 1379740_at | similar to chromosome 18 open reading frame 54 | LOC361346 |
| 1371712_at | similar to autoantigen | LOC361399 |
| 1377498_at | similar to adenosine monophosphate deaminase 2 (isoform L) | LOC362015 |
| 1377580_at | similar to mKIAA0738 protein | LOC362353 |
| 1390040_at | brain and reproductive organ-expressed protein | LOC362704 |
| 1388984_at | similar to RNA polymerase III subunit RPC2 | LOC362858 |
| 1382533_at | similar to HIV-1 Rev binding protein | LOC363266 |
| 1394991_at | similar to interleukin-1 receptor associated kinase 1 splice form 3 | LOC363520 |
| 1386580_at | similar to Wdr8 protein | LOC366515 |
| 1382029_at | similar to CDNA sequence BC017647 | LOC497960 |
| 1378587_at | similar to RIKEN cDNA 5530600A18 | LOC497981 |
| 1378966_at | LOC498061 | LOC498061 |
| 1388705_at | similar to selenoprotein SelM | LOC498398 |
| 1373569_at | similar to cDNA sequence BC018601 | LOC498404 |
| 1375718_at | Bm403207 | LOC498525 |
| 1390756_at | similar to expressed sequence AW121567 | LOC498667 |
| 1380055_at | similar to 2010004A03Rik protein | LOC498951 |
| 1398453_at | similar to cDNA sequence BC025816 | LOC498957 |
| 1382563_at | similar to hypothetical protein FLJ38944 | LOC499112 |
| 1378731_at | similar to CDNA sequence BC006909 | LOC499267 |
| 1372760_at | LOC499304 | LOC499304 |
| 1388452_at | LOC499428 | LOC499428 |
| 1393346_at | similar to RIKEN cDNA 5830436D01 | LOC499518 |
| 1382138_at | similar to hypothetical protein FLJ20433 | LOC499745 |
| 1393458_s_at | similar to PHD finger protein 14 isoform 1 | LOC500030 |
| 1392452_at | similar to PHD finger protein 14 isoform 1 | LOC500030 |
| 1396099_at | similar to Hypothetical protein MGC30714 | LOC500065 |
| 1395610_at | similar to Hypothetical protein MGC30714 | LOC500065 |
| 1397815_at | similar to ADP-ribosylation factor-like 10C | LOC500282 |
| 1394639_at | similar to CDC42 small effector 2 | LOC501691 |
| 1392257_at | hypothetical protein LOC501706 | LOC501706 |
| 1396206_at | similar to Docking protein 5 (Downstream of tyrosine kinase 5) (Protein dok- | LOC502694 |
| | 5) | |
| 1394923_at | similar to neuronal interacting factor X 1 (NIX1) | LOC502898 |
| 1367984_at | CTD-binding SR-like rA1 | LOC56081 |
| 1387373_at | myomegalin | LOC64183 |
| 1368666_a_at | calcium-independent alpha-latrotoxin receptor homolog 3 | Lphn3 |
| 1395472_at | leucine rich repeat containing 17 | Lrrc17 |
| 1375149_at | leucine rich repeat containing 4B (predicted) | Lrrc4b_predicted |
| 1395706_at | leucine rich repeat neuronal 6A (predicted) | Lrrn6a_predicted |
| 1369066_at | MAP-kinase activating death domain | Madd |
| 1392566_at | v-maf musculoaponeurotic fibrosarcoma (avian) oncogene homolog (c-maf) | Maf |
| 1367669_a_at | microtubule-associated proteins 1A/1B light chain 3 | Map1lc3b |
| 1369078_at | mitogen activated protein kinase 1 | Mapk1 |
| 1380024_at | mitogen-activated protein kinase 9 | Mapk9 |
| 1368646_at | mitogen-activated protein kinase 9 | Mapk9 |
| 1370792_at | microtubule-associated protein, RP/EB family, member 1 | Mapre1 |
| 1383114_at | serine/threonine kinase | Mark2 |
| 1375140_at | muscleblind-like 2 (predicted) | Mbnl2_predicted |
| 1387341_a_at | myelin basic protein | Mbp |
| 1369793_a_at | melanoma cell adhesion molecule | Mcam |
| 1369820_at | mcf.2 transforming sequence-like | Mcf2l |
| 1394616_at | mesoderm development candiate 2 (predicted) | Mesdc2_predicted |
| 1367796_at | Alpha-1,3-mannosyl-glycoprotein 2-beta-N-acetylglucosaminyltransferase | Mgat1 |
| 1382946_a_at | similar to Ras-related protein Rab-1B | MGC105830 |
| 1392855_x_at | similar to Ras-related protein Rab-1B | MGC105830 |
| 1398982_at | similar to Ras-related protein Rab-1B | MGC105830 |
| 1385270_s_at | similar to Ras-related protein Rab-1B | MGC105830 |
| 1393669_at | similar to Ras-related protein Rab-1B | MGC105830 |
| 1372260_at | leucine zipper domain protein | MGC116147 |
| 1372389_at | similar to immediate early response 2 | MGC72578 |
| 1375630_at | similar to NHP2-like protein 1 (High mobility group-like nuclear protein 2 | MGC72932 |
| | homolog 1) ([U4/U6.U5] tri-snRNP 15.5 kDa protein) (OTK27) | |
| 1392979_at | similar to calcyclin binding protein | MGC93921 |
| 1380072_at | similar to 2310044H10Rik protein | MGC93975 |
| 1371919_at | similar to RP2 protein, testosterone-regulated - ricefield mouse (Mus caroli) | MGC94056 |
| 1395526_at | similar to RIKEN cDNA 1700006D24 | MGC94361 |
| 1395655_at | phosphatidylinositol 4-kinase type-II beta | MGC94512 |
| 1379891_at | scotin | MGC94600 |
| 1379325_at | similar to Mkrn1 protein | MGC94941 |
| 1370129_at | meningioma expressed antigen 5 (hyaluronidase) | Mgea5 |
| 1383025_at | meningioma expressed antigen 5 (hyaluronidase) | Mgea5 |
| 1367609_at | macrophage migration inhibitory factor | Mif |
| 1375656_at | muscle, intestine and stomach expression 1 | Mist1 |
| 1369623_at | morphine related protein-1 (RefSeq = NM_022613) | Morp1 |
| 1383650_at | MAS-related G protein-coupled receptor, member B4 | Mrgprb4 |
| 1383649_a_at | MAS-related G protein-coupled receptor, member B4 | Mrgprb4 |
| 1386114_at | MAS-related G protein-coupled receptor, member B4 | Mrgprb4 |
| 1371604_at | mitochondrial ribosomal protein L34 | Mrpl34 |
| 1397604_at | membrane-spanning 4-domains, subfamily A, member 3 (predicted) | Ms4a3_predicted |
| 1368411_a_at | microtubule-associated protein 2 | Mtap2 |
| 1384314_at | mitochondrial translational initiation factor 2 | Mtif2 |
| 1394182_at | myotubularin related protein 4 (predicted) | Mtmr4_predicted |
| 1387785_at | myotrophin | Mtpn |
| 1387786_at | myotrophin | Mtpn |
| 1368232_at | mevalonate kinase | Mvk |
| 1384095_at | myosin VIIA and Rab interacting protein | Myrip |
| 1384255_at | N-ethylmaleimide-sensitive factor attachment protein, gamma (predicted) | Napg_predicted |
| 1386951_at | NADH dehydrogenase (ubiquinone) 1 alpha subcomplex 5 | Ndufa5 |
| 1395157_at | neural precursor cell expressed, developmentally down-regulated gene 4A | Nedd4a |
| 1369081_at | neuraminidase 1 | Neu1 |
| 1396092_at | NFKB inhibitor interacting Ras-like protein 1 (predicted) | Nkiras1_predicted |
| 1368895_at | neuroligin 2 | Nlgn2 |
| 1391256_at | N-myristoyltransferase 2 | Nmt2 |
| 1368929_at | nuclear protein localization 4 | Npl4 |
| 1384331_at | neoplastic progression 3 (predicted) | Npn3_predicted |
| 1386935_at | nuclear receptor subfamily 4, group A, member 1 | Nr4a1 |
| 1372032_at | neuroblastoma RAS viral (v-ras) oncogene homolog | Nras |
| 1390469_at | nurim (nuclear envelope membrane protein) | Nrm |
| 1370570_at | neuropilin 1 | Nrp1 |
| 1369689_at | N-ethylmaleimide sensitive fusion protein | Nsf |
| 1369690_at | N-ethylmaleimide sensitive fusion protein | Nsf |
| 1383957_at | nudix (nucleotide diphosphate linked moiety X)-type motif 3 (predicted) | Nudt3_predicted |
| 1387639_at | neural visinin-like Ca2+-binding protein type 2 | Nvjp2 |
| 1384144_at | nucleoredoxin (predicted) | Nxn_predicted |
| 1368962_at | neurexophilin 3 | Nxph3 |
| 1367729_at | ornithine aminotransferase | Oat |
| 1369417_a_at | opioid-binding protein/cell adhesion molecule-like | Opcml |
| 1387461_at | opioid receptor, mu 1 | Oprm1 |
| 1373442_at | amplified in osteosarcoma | Os-9 |
| 1390923_a_at | oxysterol binding protein-like 1A | Osbpl1a |
| 1369209_at | p34 protein | P34 |
| 1368958_at | protein kinase C and casein kinase substrate in neurons 1 | Pacsin1 |
| 1392890_at | platelet-activating factor acetylhydrolase, isoform Ib, alpha subunit 45 kDa | Pafah1b1 |
| 1387377_a_at | p21 (CDKN1A)-activated kinase 1 | Pak1 |
| 1396267_at | p21 (CDKN1A)-activated kinase 2 | Pak2 |
| 1383274_at | poly(rC) binding protein 3 (predicted) | Pcbp3_predicted |
| 1395441_at | poly(rC) binding protein 4 (predicted) | Pcbp4_predicted |
| 1393454_at | protocadherin 17 (predicted) | Pcdh17_predicted |
| 1368956_at | protocadherin 8 | Pcdh8 |
| 1398271_at | piccolo (presynaptic cytomatrix protein) | Pclo |
| 1369044_a_at | phosphodiesterase 4B | Pde4b |
| 1386961_at | phosphofructokinase, muscle | Pfkm |
| 1367605_at | profilin 1 | Pfn1 |
| 1367970_at | profilin 2 | Pfn2 |
| 1369473_at | phosphoglucomutase 1 | Pgm1 |
| 1379427_at | progesterone receptor membrane component 2 (predicted) | Pgrmc2_predicted |
| 1367927_at | prohibitin | Phb |
| 1369357_at | phosphorylase kinase alpha 1 | Phka1 |
| 1397572_at | phosphatidylinositol glycan, class M | Pigm |
| 1397753_at | phosphatidylinositol glycan, class Q | Pigq |
| 1379515_at | phosphatidylinositol glycan, class T (predicted) | Pigt_predicted |
| 1370531_a_at | phospholipase D1 | Pld1 |
| 1373272_at | pleckstrin homology domain containing, family A member 5 | Plekha5 |
| 1376137_at | pleckstrin homology domain containing, family B (evectins) member 2 | Plekhb2_predicted |
| | (predicted) | |
| 1384325_at | palate, lung, and nasal epithelium carcinoma associated | Plunc |
| 1370504_a_at | peripheral myelin protein 22 | Pmp22 |
| 1388717_at | protein O-fucosyltransferase 2 (predicted) | Pofut2_predicted |
| 1383037_at | polymerase (DNA-directed), delta interacting protein 2 (predicted) | Poldip2_predicted |
| 1368213_at | P450 (cytochrome) oxidoreductase | Por |
| 1368859_at | protein phosphatase 1A, magnesium dependent, alpha isoform | Ppm1a |
| 1381024_at | protein phosphatase 2 (formerly 2A), regulatory subunit A (PR 65), alpha | Ppp2r1a |
| | isoform | |
| 1375245_at | protein phosphatase 2 (formerly 2A), regulatory subunit A (PR 65), alpha | Ppp2r1a |
| | isoform | |
| 1395409_at | protein phosphatase 2A, regulatory subunit B′ (PR 53) (predicted) | Ppp2r4_predicted |
| 1395826_at | protein phosphatase 2, regulatory subunit B (B56), epsilon isoform | Ppp2r5e_predicted |
| | (predicted) | |
| 1369152_at | protein phospatase 3, regulatory subunit B, alpha isoform, type 1 | Ppp3r1 |
| 1398242_at | protein phosphatase 5, catalytic subunit | Ppp5c |
| 1369236_at | PR domain containing 4 | Prdm4 |
| 1367969_at | peroxiredoxin 6 | Prdx6 |
| 1368240_a_at | protein kinase C, beta 1 | Prkcb1 |
| 1383722_at | proline synthetase co-transcribed (predicted) | Prosc_predicted |
| 1387085_at | phosphoribosyl pyrophosphate synthetase 1 | Prps1 |
| 1368551_at | phosphoribosyl pyrophosphate synthetase 2 | Prps2 |
| 1368507_at | proteasome (prosome, macropain) subunit, alpha type 3 | Psma3 |
| 1398812_at | proteasome (prosome, macropain) subunit, beta type 1 | Psmb1 |
| 1383426_at | proline-serine-threonine phosphatase-interacting protein 1 (predicted) | Pstpip1_predicted |
| 1372663_at | phosphatidylserine synthase 2 (predicted) | Ptdss2_predicted |
| 1367851_at | prostaglandin D2 synthase | Ptgds |
| 1368527_at | prostaglandin-endoperoxide synthase 2 | Ptgs2 |
| 1369688_s_at | protein tyrosine kinase 2 beta | Ptk2b |
| 1370243_a_at | prothymosin alpha | Ptma |
| 1386892_at | parathymosin | Ptms |
| 1370574_a_at | protein tyrosine phosphatase, non-receptor type substrate 1 | Ptpns1 |
| 1370599_a_at | protein tyrosine phosphatase, receptor type, D | Ptprd |
| 1370488_a_at | protein tyrosine phosphatase, receptor type, D | Ptprd |
| 1368035_a_at | protein tyrosine phosphatase, receptor type, F | Ptprf |
| 1370214_at | parvalbumin | Pvalb |
| 1395279_at | PX domain containing serine/threonine kinase | Pxk |
| 1383072_at | muscle glycogen phosphorylase | Pygm |
| 1398825_at | RAB11B, member RAS oncogene family | Rab11b |
| 1371055_at | RAB12, member RAS oncogene family | Rab12 |
| 1370758_at | RAB15, member RAS onocogene family | Rab15 |
| 1370780_at | RAB31, member RAS oncogene family | Rab31 |
| 1369816_at | RAB3A, member RAS oncogene family | Rab3a |
| 1387641_at | RAB5A, member RAS oncogene family | Rab5a |
| 1393288_at | RAB5B, member RAS oncogene family (predicted) | Rab5b_predicted |
| 1399153_at | RAB5B, member RAS oncogene family (predicted) | Rab5b_predicted |
| 1371836_at | RAB5C, member RAS oncogene family (predicted) | Rab5c_predicted |
| 1371103_at | RAB6B, member RAS oncogene family | Rab6b |
| 1398838_at | RAB7, member RAS oncogene family | Rab7 |
| 1398780_at | Rab acceptor 1 (prenylated) | Rabac1 |
| 1372513_at | ras-related C3 botulinum toxin substrate 1 | Rac1 |
| 1395465_at | RAE1 RNA export 1 homolog (S. pombe) (predicted) | Rae1_predicted |
| 1387389_at | receptor (calcitonin) activity modifying protein 3 | Ramp3 |
| 1386900_at | ribosome associated membrane protein 4 | RAMP4 |
| 1379552_s_at | Rap2 interacting protein | Rap2ip |
| 1370373_at | RASD family, member 2 | Rasd2 |
| 1369130_at | RAS guanyl releasing protein 1 | Rasgrp1 |
| 1369129_at | RAS guanyl releasing protein 1 | Rasgrp1 |
| 1399063_at | retinoblastoma-associated factor 600 | Rbaf600 |
| 1375921_at | reticulocalbin (predicted) | Rcn_predicted |
| 1378287_at | radixin | Rdx |
| 1375542_at | radixin | Rdx |
| 1374448_at | reversion-inducing-cysteine-rich protein with kazal motifs (predicted) | Reck_predicted |
| 1369093_at | reelin | Reln |
| 1387622_at | radical fringe gene homolog (Drosophila) | Rfng |
| 1373747_at | similar to cornichon-like protein (predicted) | RGD1304930_predicted |
| 1378137_at | similar to ribosome-binding protein p34 - rat (predicted) | RGD1305092_predicted |
| 1381448_at | similar to CG6796-PA (predicted) | RGD1305145_predicted |
| 1393610_at | similar to hypothetical protein BC008163 (predicted) | RGD1305162_predicted |
| 1388959_at | similar to KIAA0153 protein (predicted) | RGD1305319_predicted |
| 1379793_at | similar to RIKEN cDNA 3110031B13 (predicted) | RGD1305356_predicted |
| 1379706_at | similar to dJ1033H22.1 (KIAA0554 protein) (predicted) | RGD1305386_predicted |
| 1388306_at | similar to RIKEN cDNA 1810042K04 (predicted) | RGD1305593_predicted |
| 1374438_at | similar to mKIAA1046 protein (predicted) | RGD1305606_predicted |
| 1390730_at | similar to 1810009O10Rik protein (predicted) | RGD1305651_predicted |
| 1390037_at | similar to chromosome 20 open reading frame 58 (predicted) | RGD1305809_predicted |
| 1395078_at | similar to semaphorin sem2 (predicted) | RGD1306079_predicted |
| 1373300_at | similar to hypothetical protein DKFZp761D0211 (predicted) | RGD1306151_predicted |
| 1392617_at | similar to hypothetical protein DKFZp761D0211 (predicted) | RGD1306151_predicted |
| 1375700_at | similar to hypothetical protein FLJ14360 (predicted) | RGD1306288_predicted |
| 1393846_at | similar to downregulated in renal cell carcinoma (predicted) | RGD1306327_predicted |
| 1395791_at | similar to mKIAA1402 protein (predicted) | RGD1306404_predicted |
| 1379795_at | similar to RIKEN cDNA 2410002O22 gene (predicted) | RGD1306583_predicted |
| 1392938_s_at | similar to C11orf17 protein (predicted) | RGD1306959_predicted |
| 1373534_at | similar to SR rich protein (predicted) | RGD1307395_predicted |
| 1396086_at | similar to RIKEN cDNA 1810014L12 (predicted) | RGD1307423_predicted |
| 1373095_at | similar to mKIAA0317 protein (predicted) | RGD1307597_predicted |
| 1373753_at | similar to RIKEN cDNA 2510010F15 (predicted) | RGD1307608_predicted |
| 1384388_at | similar to gp25L2 protein (predicted) | RGD1307627_predicted |
| 1395550_at | similar to MEGF6 (4P83) (predicted) | RGD1307670_predicted |
| 1375627_at | similar to hypothetical protein FLJ10342 (predicted) | RGD1307791_predicted |
| 1379206_at | similar to RIKEN cDNA 1110001E17 (predicted) | RGD1307901_predicted |
| 1377448_at | similar to RIKEN cDNA 1110001E17 (predicted) | RGD1307901_predicted |
| 1375648_at | similar to hypothetical protein (predicted) | RGD1307966_predicted |
| 1384615_at | similar to cisplatin resistance-associated overexpressed protein (predicted) | RGD1307981_predicted |
| 1372563_at | similar to D330021B20 protein (predicted) | RGD1308143_predicted |
| 1383837_at | similar to DNA segment, Chr 17, Wayne State University 94, expressed | RGD1308185_predicted |
| | (predicted) | |
| 1375206_at | hypothetical LOC287466 (predicted) | RGD1308212_predicted |
| 1393086_at | similar to RIKEN cDNA 5730469D23 (predicted) | RGD1308324_predicted |
| 1395334_at | similar to pancreatitis-induced protein 49 (predicted) | RGD1308600_predicted |
| 1375540_at | similar to hypothetical protein D11Ertd497e (predicted) | RGD1308696_predicted |
| 1395533_at | similar to KIAA0564 protein (predicted) | RGD1308772_predicted |
| 1373071_at | similar to RIKEN cDNA 1810054G18 (predicted) | RGD1308901_predicted |
| 1376254_at | similar to KIAA1440 protein (predicted) | RGD1308908_predicted |
| 1392621_at | similar to FKSG26 protein (predicted) | RGD1309054_predicted |
| 1393673_at | similar to HCDI protein (predicted) | RGD1309307_predicted |
| 1390315_a_at | similar to KIAA0913 protein (predicted) | RGD1309414_predicted |
| 1373814_at | similar to mKIAA1002 protein (predicted) | RGD1310066_predicted |
| 1376727_at | similar to RIKEN cDNA 2310034L04 (predicted) | RGD1310157_predicted |
| 1389302_at | similar to RIKEN cDNA D030070L09 (predicted) | RGD1310199_predicted |
| 1396137_at | similar to RIKEN cDNA C230075L19 gene (predicted) | RGD1310284_predicted |
| 1372438_at | similar to Nit protein 2 (predicted) | RGD1310494_predicted |
| 1371747_at | similar to RIKEN cDNA 2700038C09 (predicted) | RGD1310660_predicted |
| 1396708_at | similar to hypothetical protein MGC33486 (predicted) | RGD1310680_predicted |
| 1375674_at | similar to chromosome 16 open reading frame 5 (predicted) | RGD1310686_predicted |
| 1379763_at | similar to chromosome 1 open reading frame 16 (predicted) | RGD1310761_predicted |
| 1398664_at | similar to RIKEN cDNA 9130427A09 (predicted) | RGD1311016_predicted |
| 1382187_at | similar to RIKEN cDNA 2610029K21 (predicted) | RGD1311086_predicted |
| 1394760_at | similar to KIAA1409 protein (predicted) | RGD1311117_predicted |
| 1376303_a_at | membralin (predicted) | RGD1311136_predicted |
| 1373971_at | similar to PCPD protein (predicted) | RGD1311177_predicted |
| 1393826_at | similar to apolipoprotein F-like (predicted) | RGD1311384_predicted |
| 1376152_at | similar to flavoprotein oxidoreductase MICAL2 (predicted) | RGD1311773_predicted |
| 1383097_at | similar to RIKEN cDNA 2010012O05 (predicted) | RGD1311783_predicted |
| 1385302_at | similar to RIKEN cDNA 2610020H15 (predicted) | RGD1311813_predicted |
| 1391249_at | similar to DD1 (predicted) | RGD1312005_predicted |
| 1373486_at | similar to scotin (predicted) | RGD1312041_predicted |
| 1368065_at | regulator of G-protein signaling 19 interacting protein 1 | Rgs19ip1 |
| 1368144_at | regulator of G-protein signaling 2 | Rgs2 |
| 1370130_at | ras homolog gene family, member A | Rhoa |
| 1370627_at | ras homolog gene family, member V | Rhov |
| 1385790_at | ring finger protein 153 (predicted) | Rnf153_predicted |
| 1398824_at | coated vesicle membrane protein | Rnp24 |
| 1369054_at | rabphilin 3A homolog (mouse) | Rph3a |
| 1398854_at | ribosomal protein L24 | Rpl24 |
| 1376110_at | ribonuclease P 25 subunit (human) (predicted) | Rpp25_predicted |
| 1398617_at | ribosomal protein S27a | Rps27a |
| 1387888_at | ribosomal protein S9 | Rps9 |
| 1382537_at | Ras-related GTP binding C (predicted) | Rragc_predicted |
| 1369293_at | reticulon 4 receptor | Rtn4r |
| 1393404_at | reticulon 4 receptor-like 1 | Rtn4rl1 |
| 1391739_at | RUN domain containing 1 (predicted) | Rundc1_predicted |
| 1369805_at | synaptonemal complex protein SC65 | Sc65 |
| 1367959_a_at | sodium channel, voltage-gated, type I, beta polypeptide | Scn1b |
| 1387010_s_at | sodium channel, voltage-gated, type I, beta polypeptide | Scn1b |
| 1389582_at | stromal cell-derived factor 2-like 1 (predicted) | Sdf2l1_predicted |
| 1387252_at | SEC14-like 2 (S. cerevisiae) | Sec14l2 |
| 1382868_at | sema domain, transmembrane domain (TM), and cytoplasmic domain, | Sema6a_predicted |
| | (semaphorin) 6A (predicted) | |
| 1369265_at | SUMO/sentrin specific protease 2 | Senp2 |
| 1367593_at | selenoprotein W, muscle 1 | Sepw1 |
| 1368444_at | small glutamine-rich tetratricopeptide repeat (TPR)-containing, alpha | Sgta |
| 1371063_at | SH3 domain protein 2A | Sh3gl2 |
| 1368931_at | SH3 domain protein 2 C1 | Sh3gl3 |
| 1373267_at | Sh3 domain YSC-like 1 (predicted) | Sh3yl1_predicted |
| 1380435_at | soc-2 (suppressor of clear) homolog (C. elegans) (predicted) | Shoc2_predicted |
| 1390600_at | alpha-2,6-sialyltransferase ST6GalNAc IV | siat7D |
| 1372572_at | sialyltransferase 7F | Siat7F |
| 1392269_at | transcriptional regulator, SIN3A (yeast) (predicted) | Sin3a_predicted |
| 1388059_a_at | solute carrier family 11 (proton-coupled divalent metal ion transporters), | Slc11a2 |
| | member 2 | |
| 1388064_a_at | solute carrier family 1 (glial high affinity glutamate transporter), member 3 | Slc1a3 |
| 1371130_at | solute carrier family 1 (glial high affinity glutamate transporter), member 3 | Slc1a3 |
| 1369234_at | solute carrier family 20, member 2 | Slc20a2 |
| 1388221_at | solute carrier family 24 (sodium/potassium/calcium exchanger), member 3 | Slc24a3 |
| 1397647_at | solute carrier family 25 (mitochondrial carrier; ornithine transporter) member | Slc25a15_predicted |
| | 15 (predicted) | |
| 1387707_at | solute carrier family 2 (facilitated glucose transporter), member 3 | Slc2a3 |
| 1390825_at | solute carrier family 35, member B3 (predicted) | Slc35b3_predicted |
| 1381922_at | solute carrier family 5 (sodium/glucose cotransporter), member 11 | Slc5a11 |
| 1377455_at | solute carrier family 6 (neurotransmitter transporter, GABA), member 11 | Slc6a11 |
| 1369237_at | solute carrier family 6 (neurotransmitter transporter, L-proline), member 7 | Slc6a7 |
| 1369772_at | solute carrier family 6 (neurotransmitter transporter, glycine), member 9 | Slc6a9 |
| 1387280_a_at | tumor-associated protein 1 | Slc7a5 |
| 1379701_at | solute carrier family 8 (sodium/calcium exchanger), member 3 | Slc8a3 |
| 1368296_at | solute carrier organic anion transporter family, member 2b1 | Slco2b1 |
| 1392178_at | SLIT and NTRK-like family, member 1 (predicted) | Slitrk1_predicted |
| 1385011_at | SWI/SNF related, matrix associated, actin dependent regulator of chromatin, | Smarca4 |
| | subfamily a, member 4 | |
| 1384246_at | SWI/SNF related, matrix associated, actin dependent regulator of chromatin, | Smarca5_predicted |
| | subfamily a, member 5 (predicted) | |
| 1369924_at | synuclein, beta | Sncb |
| 1386282_x_at | SNF related kinase | Snrk |
| 1379275_at | sorting nexin 10 (predicted) | Snx10_predicted |
| 1396278_at | sorting nexin 11 (predicted) | Snx11_predicted |
| 1369635_at | sorbitol dehydrogenase | Sord |
| 1398274_at | spermatogenesis associated 2 | Spata2 |
| 1373636_at | sparc/osteonectin, cwcv and kazal-like domains proteoglycan 1 (predicted) | Spock1_predicted |
| 1379466_at | shadow of prion protein | Sprn |
| 1371104_at | sterol regulatory element binding factor 1 | Srebf1 |
| 1367834_at | spermidine synthase | Srm |
| 1382166_at | signal recognition particle receptor, B subunit (predicted) | Srprb_predicted |
| 1398877_at | stress-induced-phosphoprotein 1 (Hsp70/Hsp90-organizing protein) | Stip1 |
| 1388396_at | serine/threonine kinase 25 (STE20 homolog, yeast) | Stk25 |
| 1367799_at | statin-like | Stnl |
| 1387359_at | syntaxin 1A (brain) | Stx1a |
| 1386853_s_at | syntaxin 5a | Stx5a |
| 1370519_at | syntaxin binding protein 1 | Stxbp1 |
| 1370840_at | syntaxin binding protein 1 | Stxbp1 |
| 1370518_a_at | syntaxin binding protein 1 | Stxbp1 |
| 1368771_at | sulfatase 1 | Sulf1 |
| 1369627_at | synaptic vesicle glycoprotein 2b | Sv2b |
| 1369022_at | SV2 related protein | Svop |
| 1382850_at | synapsin II | Syn2 |
| 1369482_a_at | synapsin II | Syn2 |
| 1384716_at | synaptotagmin 1 | Syt1 |
| 1369135_at | synaptotagmin XI | Syt11 |
| 1387517_at | synaptotagmin 13 | Syt13 |
| 1387662_at | synaptotagmin 4 | Syt4 |
| 1368417_at | synaptotagmin 5 | Syt5 |
| 1394802_at | synaptotagmin 7 | Syt7 |
| 1381181_at | tachykinin receptor 1 | Tacr1 |
| 1377147_at | TBC1 domain family, member 7 (predicted) | Tbc1d7_predicted |
| 1380200_at | transcription factor 8 (represses interleukin 2 expression) | Tcf8 |
| 1388011_a_at | transforming growth factor, beta 2 | Tgfb2 |
| 1377596_a_at | thyroid hormone receptor associated protein 6 (predicted) | Thrap6_predicted |
| 1387983_at | thyroid hormone receptor beta | Thrb |
| 1370150_a_at | thyroid hormone responsive protein | Thrsp |
| 1387852_at | thyroid hormone responsive protein | Thrsp |
| 1369651_at | thymus cell antigen 1, theta | Thy1 |
| 1368650_at | TGFB inducible early growth response | Tieg |
| 1387495_at | transducin-like enhancer of split 4, E(spl) homolog (Drosophila) | Tle4 |
| 1392651_at | transmembrane 4 superfamily member 3 | Tm4sf3 |
| 1387850_at | transmembrane protein with EGF-like and two follistatin-like domains 1 | Tmeff1 |
| 1393418_at | tropomodulin 2 | Tmod2 |
| 1393423_at | tankyrase, TRF1-interacting ankyrin-related ADP-ribose polymerase 2 | Tnks2_predicted |
| | (predicted) | |
| 1375664_at | trinucleotide repeat containing 6 (predicted) | Tnrc6_predicted |
| 1378572_at | toll interacting protein (predicted) | Tollip_predicted |
| 1384328_at | target of myb1 homolog (chicken) (predicted) | Tom1_predicted |
| 1375420_at | tumor protein p53 inducible protein 11 (predicted) | Tp53i11_predicted |
| 1367976_at | tripeptidyl peptidase II | Tpp2 |
| 1371060_at | tripartite motif protein 23 | Trim23 |
| 1398823_at | translin-associated factor X | Tsnax |
| 1371594_at | tetratricopeptide repeat domain 11 (predicted) | Ttc11_predicted |
| 1371618_s_at | tubulin, beta 3 | Tubb3 |
| 1395719_at | Tu translation elongation factor, mitochondrial (predicted) | Tufm_predicted |
| 1385754_s_at | p105 coactivator | U83883 |
| 1385548_at | ubiquitin-conjugating enzyme E2D 1, UBC4/5 homolog (yeast) (predicted) | Ube2d1_predicted |
| 1369617_at | ubiquitin-conjugating enzyme E2N (homologous to yeast UBC13) | Ube2n |
| 1385615_at | ubiquitin conjugation factor E4 A | Ube4a |
| 1371188_a_at | upstream binding transcription factor, RNA polymerase I | Ubtf |
| 1369977_at | ubiquitin carboxy-terminal hydrolase L1 | Uchl1 |
| 1382257_at | uridine monophosphate synthetase (predicted) | Umps_predicted |
| 1369394_at | unc-5 homolog A (C. elegans) | Unc5a |
| 1387624_at | upstream transcription factor 1 | Usf1 |
| 1388088_a_at | upstream transcription factor 2 | Usf2 |
| 1387703_a_at | ubiquitin specific protease 2 | Usp2 |
| 1376127_at | UDP-glucuronate decarboxylase 1 | Uxs1 |
| 1369597_at | vesicle-associated membrane protein, associated protein B and C | Vapb |
| 1386909_a_at | voltage-dependent anion channel 1 | Vdac1 |
| 1370549_at | vacuolar protein sorting 45 (yeast) | Vps45 |
| 1369667_at | vacuolar protein sorting 52 (yeast) | Vps52 |
| 1368853_at | visinin-like 1 | Vsnl1 |
| 1397675_at | eukaryotic translation initiation factor 4H | Wbscr1 |
| 1381070_at | WD repeat domain 36 (predicted) | Wdr36_predicted |
| 1369344_at | WD repeat domain 7 | Wdr7 |
| 1386807_at | wingless-related MMTV integration site 2 | Wnt2 |
| 1383451_at | WD SOCS-box protein 2 | Wsb2 |
| 1385343_at | X-box binding protein 1 | Xbp1 |
| 1375903_a_at | YY1 associated factor 2 (predicted) | Yaf2_predicted |
| 1376444_at | YY1 transcription factor | Yy1 |
| 1388731_at | zinc binding alcohol dehydrogenase, domain containing 2 (predicted) | Zadh2_predicted |
| 1384783_at | zinc finger protein 161 | Zfp161 |
| 1380106_at | zinc finger protein 162 | Zfp162 |
| 1380416_at | zinc finger protein 191 | Zfp191 |
| 1380529_at | zinc finger protein 207 (predicted) | Zfp207_predicted |
| 1387512_at | zinc finger protein 238 | Zfp238 |
| 1369501_at | zinc finger protein 260 | Zfp260 |
| 1385658_at | zinc finger protein 313 | Znf313 |
| 1388919_at | zinc finger protein 541 (predicted) | Znf541_predicted |
| 1388130_at | zyxin | Zyx |
| 1390019_at | H3 histone, family 3B | H3f3b |
| 1380025_at | Rattus norvegicus catechol-O-methyltransferase (Comt), mRNA. | Comt |
| 1396820_at | histone deacetylase 1 (predicted) | Hdac1_predicted |
| 1389787_at | serum response factor (predicted) | Srf_predicted |

Genes and gene fragments identified as changing significantly in animals that were reared in social isolation, relative to social control animals on postnatal day 80 (P80) are provided in Table 6.

TABLE 6

| Fragment Name | Gene Name | Gene Symbol |
|---|---|---|
| 1369997_at | (dishevelled, dsh homolog 1 (Drosophila), limitrin) | (1200013a08rik, Dvl1) |
| 1392108_at | (ATP-binding cassette, sub-family C (CFTR/MRP), member 3, aldehyde | (Abcc3, Aox3, Ccl9_predicted, |
| | oxidase 3, chemokine (C-C motif) ligand 9 (predicted), enoyl-Coenzyme A, | Ehhadh, Gmpr2, Tnfrsf1b) |
| | hydratase/3-hydroxyacyl Coenzyme A dehydrogenase, guanosine | |
| | monophosphate reductase 2, tumor necrosis factor recept | |
| 1372087_at | (a disintegrin and metalloproteinase domain 17 (tumor necrosis factor, alpha, | (Adam17, harpb64) |
| | converting enzyme), hypertrophic agonist responsive protein) | |
| 1385577_at | (5 nucleotidase, 6-pyruvoyl-tetrahydropterin synthase, Ras homolog enriched | (Adh6_predicted, Agtrap, Apaf1, |
| | in brain like 1, alcohol dehydrogenase 6 (class V) (predicted), angiotensin II | Cdkl1_predicted, Cln2, Galm, |
| | receptor-associated protein, apoptotic peptidase activating factor 1, ceroid- | Gcat_predicted, Hspa1b, Keg1, |
| | lipofuscinosis, neurona | LOC293989, LOC499300, |
| | | MGC95001, Nt5, Pgsg, Pts, |
| | | Rhebl1, Rps6kb2_predicted, |
| | | Tsarg1, Ttc4_predicted, sag) |
| 1383377_at | (ATP synthase, H+ transporting, mitochondrial F0 complex, subunit F6, GA | (Atp5j, Gabpa_predicted) |
| | repeat binding protein, alpha (predicted)) | |
| 1370913_at | (Best5 protein, hypothetical gene supported by NM_138881) | (Best5, LOC497812) |
| 1370892_at | (complement component 4, gene 2, complement component 4a) | (C4-2, C4a) |
| 1370891_at | (CD48 antigen, similar to cytokine receptor related protein 4) | (Cd48, Cytor4) |
| 1372977_at | (atlastin-like, cyclin-dependent kinase-like 1 (CDC2-related kinase) | (Cdkl1_predicted, LOC362750, |
| | (predicted), similar to mitogen-activated protein kinase kinase kinase kinase | LOC503027) |
| | 5 isoform 2) | |
| 1387305_s_at | (cytochrome P450, family 11, subfamily B, polypeptide 2, cytochrome P450, | (Cyp11b1, Cyp11b2) |
| | subfamily 11B, polypeptide 1) | |
| 1391661_at | (glycerol kinase, hypothetical gene supported by NM_024381) | (Gyk, LOC497845) |
| 1371102_x_at | (beta-glo, hemoglobin beta chain complex) | (Hbb, MGC72973) |
| 1368255_at | (hypothetical gene supported by NM_017354, neurotrimin) | (Hnt, LOC360435) |
| 1388850_at | (heat shock 90 kDa protein 1, alpha-like 3 (predicted), heat shock protein 1, | (Hspca, Hspcal3_predicted) |
| | alpha) | |
| 1387995_a_at | (interferon induced transmembrane protein 2 (1-8D), interferon induced | (Ifitm2, Ifitm3) |
| | transmembrane protein 3) | |
| 1376758_at | (LOC498665, inhibitor of growth family, member 1 (predicted)) | (Ing1_predicted, LOC498665) |
| 1369035_a_at | (hypothetical gene supported by NM_013192, potassium inwardly-rectifying | (Kcnj6, LOC497678) |
| | channel, subfamily J, member 6) | |
| 1397130_at | (mitogen activated protein kinase 10, similar to binding protein) | (LOC293702, Mapk10) |
| 1381030_at | (similar to step II splicing factor SLU7; DNA segment, Chr 11, ERATO Doi | (LOC303057, Slu7) |
| | 730, expressed; DNA segment, Chr 3, Brigham & Womens Genetics 0878 | |
| | expressed, step II splicing factor SLU7 (S. cerevisiae)) | |
| 1372604_at | (hypothetical protein LOC503164, similar to RIKEN cDNA 2210421G13) | (LOC315106, LOC503164) |
| 1388164_at | (MHC class I RT1.O type 149 processed pseudogene, RT1 class I, T24, | (LOC360231, LOC499402, RT1- |
| | gene 4, RT1 class Ib, locus H2-TL-like (S2), RT1 class Ib, locus S3, similar to | 149, RT1-S2, RT1-S3) |
| | class I histocompatibility antigen alpha chain - cotton-top tamarin) | |
| 1395691_at | (similar to phosphoseryl-tRNA kinase, zinc finger protein, subfamily 1A, 5 | (LOC361661, |
| | (predicted)) | Zfpn1a5_predicted) |
| 1368993_at | (hypothetical gene supported by NM_020088, odd Oz/ten-m homolog 2 | (LOC497664, Odz2) |
| | (Drosophila)) | |
| 1387071_a_at | (hypothetical gene supported by NM_017212, microtubule-associated protein | (LOC497674, Mapt) |
| | tau) | |
| 1398861_at | (hypothetical gene supported by NM_021579, nuclear RNA export factor 1) | (LOC497739, Nxf1) |
| 1371230_x_at | (serine protease inhibitor, Kazal type 1, similar to ATPase inhibitor) | (LOC497807, Spink1) |
| 1368261_at | (hypothetical gene supported by NM_053817, neurexin 3) | (LOC497817, Nrxn3) |
| 1392316_at | (LOC498458, protein tyrosine phosphatase, receptor type, G) | (LOC498458, Ptprg) |
| 1373992_at | (similar to MGC108823 protein, similar to interferon-inducible GTPase) | (LOC498872, MGC108823) |
| 1369896_s_at | (LOC499013, RNA binding motif protein 16) | (LOC499013, Rbm16) |
| 1373782_a_at | (LOC499769, similar to LOC495800 protein) | (LOC499769, LOC499770) |
| 1373666_at | (Rap guanine nucleotide exchange factor (GEF) 5, similar to Rap guanine | (LOC500748, Rapgef5) |
| | nucleotide exchange factor 5 (Guanine nucleotide exchange factor for Rap1) | |
| | (M-Ras-regulated Rap GEF) (MR-GEF)) | |
| 1371017_at | (T-cell receptor gamma chain, similar to TCR V gamma 3) | (LOC502132, Tcrg) |
| 1369138_a_at | (parkin, similar to mature parasite-infected erythrocyte surface antigen like | (LOC502231, Park2) |
| | precursor (2N179)) | |
| 1371152_a_at | (2′,5′-oligoadenylate synthetase 1, 40/46 kDa, 2′-5′ oligoadenylate synthetase | (Oas1, Oas1i, Oas1k) |
| | 1I, 2′-5′ oligoadenylate synthetase 1K) | |
| 1378934_at | (zinc responsive protein ZD10B, zinc responsive protein Zd10A) | (ZD10B, Zd10A) |
| 1368330_at | apoptosis antagonizing transcription factor | Aatf |
| 1388153_at | acyl-CoA synthetase long-chain family member 1 | Acsl1 |
| 1388179_at | activin receptor IIB | Acvr2b |
| 1383418_at | a disintegrin and metalloprotease domain 11 (predicted) | Adam11_predicted |
| 1374419_at | adenylate cyclase 9 (predicted) | Adcy9_predicted |
| 1382981_at | Abelson helper integration site 1 | Ahi1 |
| 1368558_s_at | allograft inflammatory factor 1 | Aif1 |
| 1367555_at | albumin | Alb |
| 1368776_at | arachidonate 5-lipoxygenase | Alox5 |
| 1367775_at | alpha-methylacyl-CoA racemase | Amacr |
| 1381042_at | anaphase promoting complex subunit 10 (predicted) | Anapc10_predicted |
| 1374163_at | anaphase promoting complex subunit 4 (predicted) | Anapc4_predicted |
| 1395313_s_at | annexin A3 | Anxa3 |
| 1376413_at | amyloid beta (A4) precursor protein-binding, family A, member 1 | Apba1 |
| 1398258_at | apolipoprotein D | Apod |
| 1395053_at | androgen-induced proliferation inhibitor (predicted) | Aprin_predicted |
| 1392864_at | Rho GTPase activating protein 5 (predicted) | Arhgap5_predicted |
| 1370002_at | Rho guanine nucleotide exchange factor (GEF) 1 | Arhgef1 |
| 1368916_at | argininosuccinate lyase | Asl |
| 1368701_at | ATPase, Na+/K+ transporting, alpha 3 polypeptide | Atp1a3 |
| 1386426_at | ATPase, Ca++ transporting, plasma membrane 1 | Atp2b1 |
| 1387126_at | ATPase, Ca++-sequestering | Atp2c1 |
| 1367724_a_at | ATPase, H+ transporting, V0 subunit E isoform 1 | Atp6v0e1 |
| 1373087_at | axotrophin (predicted) | Axot_predicted |
| 1379419_at | DNA sequence AY228474 | AY228474 |
| 1392421_at | bromodomain adjacent to zinc finger domain, 2B (predicted) | Baz2b_predicted |
| 1371391_at | bone morphogenetic protein 6 | Bmp6 |
| 1386994_at | B-cell translocation gene 2, anti-proliferative | Btg2 |
| 1397151_at | calcium channel, voltage-dependent, alpha 2/delta 3 subunit | Cacna2d3 |
| 1367889_at | calcium/calmodulin-dependent protein kinase I | Camk1 |
| 1370438_at | C-terminal PDZ domain ligand of neuronal nitric oxide synthase | Capon |
| 1370810_at | cyclin D2 | Ccnd2 |
| 1374540_at | cell division cycle associated 7 (predicted) | Cdca7_predicted |
| 1392140_at | cadherin 11 | Cdh11 |
| 1368887_at | cadherin 22 | Cdh22 |
| 1368545_at | CASP8 and FADD-like apoptosis regulator | Cflar |
| 1380063_at | cholesterol 25-hydroxylase (predicted) | Ch25h_predicted |
| 1370991_at | camello-like 3 | Cml3 |
| 1381294_at | cyclin M1 (predicted) | Cnnm1_predicted |
| 1394008_x_at | ciliary neurotrophic factor receptor | Cntfr |
| 1370376_a_at | cold shock domain protein A | Csda |
| 1367631_at | connective tissue growth factor | Ctgf |
| 1397217_at | CUG triplet repeat, RNA-binding protein 2 | Cugbp2 |
| 1369068_at | cullin 5 | Cul5 |
| 1387913_at | cytochrome P450, family 2, subfamily d, polypeptide 22 | Cyp2d22 |
| 1379855_at | deleted in colorectal carcinoma | Dcc |
| 1386535_at | DEAD (Asp-Glu-Ala-Asp) box polypeptide 3, X-linked | Ddx3x |
| 1380807_at | discs, large homolog 1 (Drosophila) | Dlgh1 |
| 1375139_at | discs, large homolog 2 (Drosophila) | Dlgh2 |
| 1370073_at | protein kinase inhibitor p58 | Dnajc3 |
| 1398803_at | dynein, cytoplasmic, heavy chain 1 | Dnch1 |
| 1383895_at | dynein, cytoplasmic, light chain 2B (predicted) | Dncl2b_predicted |
| 1388134_at | eukaryotic translation elongation factor 1 delta (guanine nucleotide exchange | Eef1d |
| | protein) | |
| 1368867_at | GERp95 | Eif2c2 |
| 1370542_a_at | E74-like factor 1 | Elf1 |
| 1369453_at | Epsin 1 | Epn1 |
| 1387964_a_at | ERO1-like (S. cerevisiae) | Ero1l |
| 1372823_at | family with sequence similarity 36, member A (predicted) | Fam36a_predicted |
| 1383354_a_at | F-box only protein 33 (predicted) | Fbxo33_predicted |
| 1384829_at | fibroblast growth factor receptor 3 | Fgfr3 |
| 1383904_at | fibronigen-like protein 1 | Fgl1 |
| 1379263_at | fukutin related protein (predicted) | Fkrp_predicted |
| 1375043_at | FBJ murine osteosarcoma viral oncogene homolog | Fos |
| 1390936_at | FXYD domain-containing ion transport regulator 3 | Fxyd3 |
| 1382314_at | interferon, alpha-inducible protein (clone IFI-15K) (predicted) | G1p2_predicted |
| 1388792_at | growth arrest and DNA-damage-inducible 45 gamma (predicted) | Gadd45g_predicted |
| 1378900_at | GCN5 general control of amino acid synthesis-like 2 (yeast) (predicted) | Gcn5l2_predicted |
| 1369640_at | gap junction membrane channel protein alpha 1 | Gja1 |
| 1387906_a_at | GNAS complex locus | Gnas |
| 1375705_at | guanine nucleotide binding protein, beta 1 | Gnb1 |
| 1387670_at | glycerol-3-phosphate dehydrogenase 2 | Gpd2 |
| 1394578_at | glutamate receptor, ionotropic, 2 | Gria2 |
| 1393995_at | glutamate receptor, ionotropic, 2 | Gria2 |
| 1389574_at | general transcription factor IIIC, polypeptide 2, beta 110 kDa | Gtf3c2 |
| 1376285_at | GULP, engulfment adaptor PTB domain containing 1 (predicted) | Gulp1_predicted |
| 1367759_at | H1 histone family, member 0 | H1f0 |
| 1378614_at | Huntington disease gene homolog | Hdh |
| 1374599_at | hect (homologous to the E6-AP (UBE3A) carboxyl terminus) domain and | Herc1_predicted |
| | RCC1 (CHC1)-like domain (RLD) 1 (predicted) | |
| 1394746_at | hect (homologous to the E6-AP (UBE3A) carboxyl terminus) domain and | Herc1_predicted |
| | RCC1 (CHC1)-like domain (RLD) 1 (predicted) | |
| 1388309_at | high mobility group AT-hook 1 | Hmga1 |
| 1371336_at | hematological and neurological expressed sequence 1 | Hn1 |
| 1371510_at | HCF-1 beta-propeller interacting protein | Hpip |
| 1371255_at | Harvey rat sarcoma viral (v-Ha-ras) oncogene homolog | Hras |
| 1368416_at | integrin binding sialoprotein | Ibsp |
| 1375532_at | Inhibitor of DNA binding 2, dominant negative helix-loop-helix protein | Id2 |
| 1385923_at | inhibitor of DNA binding 4 | Idb4 |
| 1381014_at | interferon-induced protein 44 (predicted) | Ifi44_predicted |
| 1384180_at | interferon-induced protein with tetratricopeptide repeats 2 (predicted) | Ifit2_predicted |
| 1387625_at | insulin-like growth factor binding protein 6 | Igfbp6 |
| 1390715_at | insulin-like growth factor binding protein-like 1 (predicted) | Igfbpl1_predicted |
| 1370331_at | interleukin 11 receptor, alpha chain 1 | Il11ra1 |
| 1371148_s_at | internexin, alpha | Inexa |
| 1383564_at | interferon regulatory factor 7 (predicted) | Irf7_predicted |
| 1383448_at | interferon dependent positive acting transcription factor 3 gamma (predicted) | Isgf3g_predicted |
| 1374627_at | interferon dependent positive acting transcription factor 3 gamma (predicted) | Isgf3g_predicted |
| 1387788_at | Jun-B oncogene | Junb |
| 1396701_at | kalirin, RhoGEF kinase | Kalrn |
| 1381010_at | potassium voltage gated channel, Shal-related family, member 2 | Kcnd2 |
| 1370970_at | potassium inwardly-rectifying channel, subfamily J, member 14 | Kcnj14 |
| 1397599_at | potassium voltage-gated channel, subfamily Q, member 3 | Kcnq3 |
| 1392717_at | kinesin family member 1B | Kif1b |
| 1380172_at | kinesin family member 5C (predicted) | Kif5c_predicted |
| 1367880_at | laminin, beta 2 | Lamb2 |
| 1387946_at | lectin, galactoside-binding, soluble, 3 binding protein | Lgals3bp |
| 1389329_at | lectin, galactose binding, soluble 8 | Lgals8 |
| 1381798_at | LIM domain only protein 7 | LMO7 |
| 1389560_at | Vps24p protein | LOC282834 |
| 1370853_at | CaM-kinase II inhibitor alpha | LOC287005 |
| 1375526_at | similar to novel protein of unknown function (DUF423) family member | LOC287442 |
| 1391416_at | similar to ankyrin repeat domain protein 17 isoform b | LOC289521 |
| 1392723_at | similar to ankyrin repeat domain protein 17 isoform b | LOC289521 |
| 1375046_at | hypothetical protein LOC289786 | LOC289786 |
| 1376562_at | similar to Traf2 and NCK interacting kinase, splice variant 4 | LOC294917 |
| 1385713_at | similar to NAKAP95 | LOC299569 |
| 1390375_at | similar to NAKAP95 | LOC299569 |
| 1382111_at | similar to RIKEN cDNA 2010001H14 | LOC302554 |
| 1378730_at | similar to BCL6 co-repressor-like 1 | LOC302810 |
| 1378347_at | similar to hypothetical protein FLJ34512 | LOC302996 |
| 1383920_at | similar to aminomethyltransferase | LOC306586 |
| 1371389_at | hypothetical LOC306766 | LOC306766 |
| 1396232_at | similar to hypothetical protein FLJ25333 | LOC309925 |
| 1375539_at | similar to T-Brain-1 | LOC311078 |
| 1389034_at | similar to ubiquitin specific protease UBP43 | LOC312688 |
| 1396803_at | similar to THO complex 2 | LOC313308 |
| 1372496_at | similar to hypothetical protein MGC19604 | LOC313786 |
| 1396567_at | similar to Set alpha isoform | LOC317165 |
| 1383673_at | similar to Nap1l2 | LOC317247 |
| 1384550_at | similar to RIKEN cDNA 1810030O07 | LOC317344 |
| 1374494_at | similar to testis-specific chromodomain Y-like protein | LOC361237 |
| 1381966_at | similar to transcription factor 1 | LOC361475 |
| 1389099_at | similar to hypothetical protein MGC51082 | LOC361519 |
| 1372075_at | similar to dJ862K6.2.2 (splicing factor, arginine/serine-rich 6 (SRP55- | LOC362264 |
| | 2)(isoform 2)) | |
| 1374421_at | similar to Williams-Beuren syndrome deletion transcript 9 homolog | LOC368002 |
| 1372324_at | similar to thyroid hormone receptor interactor 3 | LOC497975 |
| 1378966_at | LOC498061 | LOC498061 |
| 1396530_at | Ab2-093 | LOC498084 |
| 1373575_at | similar to NADH dehydrogenase (ubiquinone) Fe—S protein 2 | LOC498279 |
| 1398390_at | similar to Small inducible cytokine B13 precursor (CXCL13) (B lymphocyte | LOC498335 |
| | chemoattractant) (CXC chemokine BLC) | |
| 1380728_at | similar to collapsin response mediator protein-2A | LOC498539 |
| 1381556_at | similar to BC013672 protein | LOC498673 |
| 1375821_at | similar to ankyrin repeat domain 26 | LOC498767 |
| 1381230_at | similar to RIKEN cDNA 2610039E05 | LOC498963 |
| 1391748_at | similar to Myb protein P42POP | LOC499090 |
| 1386793_at | similar to zinc finger protein 61 | LOC499094 |
| 1373410_at | similar to MADS box transcription enhancer factor 2, polypeptide C (myocyte | LOC499497 |
| | enhancer factor 2C) | |
| 1392070_at | similar to Lix1 homolog (mouse) like | LOC499677 |
| 1376693_at | similar to OEF2 | LOC500011 |
| 1393201_at | similar to lymphoid-restricted membrane protein | LOC500361 |
| 1393866_at | similar to WSL-1-like protein | LOC500592 |
| 1397700_x_at | LOC500721 | LOC500721 |
| 1378099_at | LOC500721 | LOC500721 |
| 1398716_at | LOC500721 | LOC500721 |
| 1381758_at | similar to MDM2 Binding protein | LOC500870 |
| 1380184_at | similar to TAFA5 | LOC500915 |
| 1374028_at | similar to CDNA sequence BC024479 | LOC500974 |
| 1398659_at | similar to PSST739 protein | LOC501231 |
| 1399041_at | similar to OPA3 protein | LOC502305 |
| 1371405_at | similar to hypothetical protein MGC52110 | LOC503252 |
| 1367984_at | CTD-binding SR-like rA1 | LOC56081 |
| 1389510_at | Ly1 antibody reactive clone (predicted) | Lyar_predicted |
| 1398834_at | mitogen activated protein kinase kinase 2 | Map2k2 |
| 1386959_a_at | mitogen activated protein kinase kinase 5 | Map2k5 |
| 1375673_at | mitogen activated protein kinase kinase kinase 1 | Map3k1 |
| 1368710_at | serine/threonine kinase | Mark2 |
| 1387834_at | megakaryocyte-associated tyrosine kinase | Matk |
| 1379456_at | mitochondrial carrier triple repeat 1 (predicted) | Mcart1_predicted |
| 1376986_at | methyltransferase-like 3 (predicted) | Mettl3_predicted |
| 1368071_at | Mg87 protein | Mg87 |
| 1372389_at | similar to immediate early response 2 | MGC72578 |
| 1372967_at | RAC/CDC42 exchange factor | MGC72605 |
| 1373023_at | similar to RIKEN cDNA 2010200I23 | MGC94262 |
| 1377935_at | similar to Protein C3orf4 homolog | MGC94479 |
| 1379891_at | scotin | MGC94600 |
| 1395223_at | similar to hypothetical protein MGC35097 | MGC94736 |
| 1372599_at | microsomal glutathione S-transferase 2 (predicted) | Mgst2_predicted |
| 1385422_at | myeloid/lymphoid or mixed-lineage leukemia 5 (trithorax homolog, | Mll5_predicted |
| | Drosophila) (predicted) | |
| 1375703_at | myeloid/lymphoid or mixed-lineage leukemia 5 (trithorax homolog, | Mll5_predicted |
| | Drosophila) (predicted) | |
| 1369825_at | matrix metallopeptidase 2 | Mmp2 |
| 1388853_at | mitochondrial ribosomal protein L54 (predicted) | Mrpl54_predicted |
| 1371015_at | myxovirus (influenza virus) resistance 1 | Mx1 |
| 1387283_at | myxovirus (influenza virus) resistance 2 | Mx2 |
| 1376648_at | v-myc myelocytomatosis viral related oncogene, neuroblastoma derived | Mycn |
| | (avian) | |
| 1370174_at | myeloid differentiation primary response gene 116 | Myd116 |
| 1368450_at | myosin Va | Myo5a |
| 1387866_at | myosin IXb | Myo9b |
| 1373304_at | alpha-N-acetylglucosaminidase | Naglu |
| 1396264_at | nuclear cap binding protein subunit 2 (predicted) | Ncbp2_predicted |
| 1387577_at | neurogenic differentiation 2 | Neurod2 |
| 1388167_at | nuclear factor I/B | Nfib |
| 1371202_a_at | nuclear factor I/B | Nfib |
| 1395083_at | neuro-oncological ventral antigen 1 | Nova1 |
| 1387099_at | natriuretic peptide receptor 2 | Npr2 |
| 1397004_at | nuclear receptor subfamily 3, group C, member 1 | Nr3c1 |
| 1372032_at | neuroblastoma RAS viral (v-ras) oncogene homolog | Nras |
| 1369404_a_at | neurexin 1 | Nrxn1 |
| 1388340_at | Ns5atp9 protein | Ns5atp9 |
| 1385280_at | nucleotide binding protein 1 (predicted) | Nubp1_predicted |
| 1368466_a_at | outer dense fiber of sperm tails 2 | Odf2 |
| 1379481_at | poly(A) binding protein, nuclear 1 | Pabpn1 |
| 1392480_at | poly(A) binding protein, nuclear 1 | Pabpn1 |
| 1368958_at | protein kinase C and casein kinase substrate in neurons 1 | Pacsin1 |
| 1376247_at | phosphoenolpyruvate carboxykinase 2 (mitochondrial) (predicted) | Pck2_predicted |
| 1388020_a_at | phosphodiesterase 1C | Pde1c |
| 1398472_at | progressive external ophthalmoplegia 1 (predicted) | Peo1_predicted |
| 1372923_at | peroxisomal biogenesis factor 11b (predicted) | Pex11b_predicted |
| 1370833_at | peroxin 2 | Pex2 |
| 1368264_at | peroxisomal biogenesis factor 6 | Pex6 |
| 1370447_at | phospholipase B | Phlpb |
| 1369177_at | phosphatidylinositol 4-kinase type 2 alpha | Pi4k2a |
| 1384558_at | placenta-specific 9 (predicted) | Plac9_predicted |
| 1381133_at | phospholipase C, beta 1 | Plcb1 |
| 1398899_at | polymerase (RNA) II (DNA directed) polypeptide C (predicted) | Polr2c_predicted |
| 1379832_at | polymerase (RNA) II (DNA directed) polypeptide D (predicted) | Polr2d_predicted |
| 1386971_at | protein phosphatase 1, regulatory subunit 10 | Ppp1r10 |
| 1376938_at | protein phosphatase 2 (formerly 2A), regulatory subunit B (PR 52), alpha | Ppp2r2a |
| | isoform | |
| 1384815_at | protein phosphatase 3, catalytic subunit, alpha isoform | Ppp3ca |
| 1368507_at | proteasome (prosome, macropain) subunit, alpha type 3 | Psma3 |
| 1376069_at | proteasome (prosome, macropain) 26S subunit, non-ATPase, 11 (predicted) | Psmd11_predicted |
| 1374803_at | proteasome (prosome, macropain) 26S subunit, non-ATPase, 11 (predicted) | Psmd11_predicted |
| 1388430_at | prostate tumor over expressed gene 1 (predicted) | Ptov1_predicted |
| 1370181_at | RAB4A, member RAS oncogene family | Rab4a |
| 1367981_at | rabaptin 5 | Rabep1 |
| 1390941_at | retinoblastoma binding protein 6 (predicted) | Rbbp6_predicted |
| 1370356_at | RNA binding motif protein 10 | Rbm10 |
| 1396401_at | RNA binding motif protein 17 (predicted) | Rbm17_predicted |
| 1395271_at | RNA binding motif protein 27 (predicted) | Rbm27_predicted |
| 1383164_at | replication factor C (activator 1) 3 (predicted) | Rfc3_predicted |
| 1377532_at | similar to Hepatocellular carcinoma-associated antigen 58 homolog | RGD1305020_predicted |
| | (predicted) | |
| 1372795_at | similar to hypothetical protein FLJ20511 (predicted) | RGD1305127_predicted |
| 1381448_at | similar to CG6796-PA (predicted) | RGD1305145_predicted |
| 1385862_at | similar to RIKEN cDNA 4930438O05 (predicted) | RGD1305615_predicted |
| 1397513_at | hypothetical LOC294883 (predicted) | RGD1305844_predicted |
| 1395448_at | similar to hypothetical protein FLJ13511 (predicted) | RGD1305857_predicted |
| 1393326_s_at | similar to Pseudoautosomal GTP-binding protein-like protein (predicted) | RGD1305954_predicted |
| 1395637_at | similar to aspartyl beta-hydroxylase; calsequestrin-binding protein; | RGD1306020_predicted |
| | 3110001L23Rik (predicted) | |
| 1380228_at | similar to hypothetical protein MGC47816 (predicted) | RGD1306880_predicted |
| 1379285_at | similar to 5830458K16Rik protein (predicted) | RGD1306974_predicted |
| 1389203_at | hypothetical LOC287306 (predicted) | RGD1307036_predicted |
| 1397848_at | similar to RIKEN cDNA 6330406I15 (predicted) | RGD1307396_predicted |
| 1373920_at | similar to 106 kDa O-GlcNAc transferase-interacting protein (predicted) | RGD1307844_predicted |
| 1383265_at | similar to RIKEN cDNA 4930451A13 (predicted) | RGD1307925_predicted |
| 1377996_at | hypothetical LOC304497 (predicted) | RGD1308037_predicted |
| 1389769_at | similar to KIAA1440 protein (predicted) | RGD1308908_predicted |
| 1391346_at | similar to FKSG26 protein (predicted) | RGD1309054_predicted |
| 1377950_at | similar to interferon-inducible GTPase (predicted) | RGD1309362_predicted |
| 1376221_at | similar to DNA segment, Chr 7, ERATO Doi 462, expressed (predicted) | RGD1309393_predicted |
| 1373814_at | similar to mKIAA1002 protein (predicted) | RGD1310066_predicted |
| 1372433_at | similar to CG11030-PA (predicted) | RGD1310211_predicted |
| 1381513_at | similar to RIKEN cDNA 1300017J02 (predicted) | RGD1310507_predicted |
| 1374139_at | similar to cerebellar degeneration-related 2 (predicted) | RGD1310578_predicted |
| 1393460_at | similar to GARP protein precursor (Garpin) (Glycoprotein A repetitions | RGD1310771_predicted |
| | predominant) (predicted) | |
| 1388790_at | similar to hypothetical protein D5Ertd33e (predicted) | RGD1310857_predicted |
| 1376612_at | similar to RIKEN cDNA 1810033A06 (predicted) | RGD1311144_predicted |
| 1372784_at | similar to Muf1-pending protein (predicted) | RGD1311221_predicted |
| 1392797_at | similar to CGI-41 protein (predicted) | RGD1311265_predicted |
| 1377995_at | similar to hypothetical protein DKFZp761D0211 (predicted) | RGD1311484_predicted |
| 1376136_at | MIRO2 protein | Rhot2 |
| 1381967_at | RNA-binding region (RNP1, RRM) containing 2 (predicted) | Rnpc2_predicted |
| 1379737_a_at | RNA-binding region (RNP1, RRM) containing 2 (predicted) | Rnpc2_predicted |
| 1384654_at | roundabout homolog 1 (Drosophila) | Robo1 |
| 1377029_at | RAR-related orphan receptor alpha (predicted) | Rora_predicted |
| 1375788_at | ribosomal protein L7 | Rpl7 |
| 1389051_at | retinoid X receptor beta | Rxrb |
| 1373188_at | sodium channel, voltage-gated, type IV, beta | Scn4b |
| 1372180_at | syndecan 3 | Sdc3 |
| 1371706_at | serologically defined colon cancer antigen 3 (predicted) | Sdccag3_predicted |
| 1368109_at | sialyltransferase 9 (CMP-NeuAc: lactosylceramide alpha-2,3-sialyltransferase) | Siat9 |
| 1389747_at | solute carrier family 26, member 8 (predicted) | Slc26a8_predicted |
| 1368483_a_at | slit homolog 1 (Drosophila) | Slit1 |
| 1374391_at | sarcolipin (predicted) | Sln_predicted |
| 1382020_at | sperm associated antigen 9 (predicted) | Spag9_predicted |
| 1395014_at | spastic paraplegia 7 homolog (human) | Spg7 |
| 1375374_at | sequestosome 1 | Sqstm1 |
| 1389030_a_at | Rous sarcoma oncogene | Src |
| 1375459_at | serine/arginine-rich protein specific kinase 2 (predicted) | Srpk2_predicted |
| 1378431_at | serine/arginine-rich protein specific kinase 2 (predicted) | Srpk2_predicted |
| 1398940_at | serine/arginine repetitive matrix 2 (predicted) | Srrm2_predicted |
| 1373670_at | signal transducer and activator of transcription 2 (predicted) | Stat2_predicted |
| 1369047_at | sulfotransferase family 1D, member 1 | Sult1d1 |
| 1376690_at | SRB7 (supressor of RNA polymerase B) homolog (S. cerevisiae) (predicted) | Surb7_predicted |
| 1371791_at | surfeit 1 | Surf1 |
| 1379635_at | SYAP1 protein | Syap1 |
| 1378886_x_at | Nuclear envelope spectrin repeat protein 1 | Syne1 |
| 1396512_at | synaptogyrin 1 | Syngr1 |
| 1389778_a_at | transcription elongation factor B (SIII), polypeptide 3 | Tceb3 |
| 1397286_at | transcription factor 4 | Tcf4 |
| 1377340_at | tissue factor pathway inhibitor 2 | Tfpi2 |
| 1370323_at | thimet oligopeptidase 1 | Thop1 |
| 1370691_a_at | thyroid hormone receptor alpha | Thra |
| 1397692_at | cytotoxic granule-associated RNA binding protein 1 (predicted) | Tia1_predicted |
| 1381557_at | TGFB inducible early growth response 3 (predicted) | Tieg3_predicted |
| 1390125_at | transmembrane 9 superfamily member 1 (predicted) | Tm9sf1_predicted |
| 1380060_at | DNA topoisomerase I, mitochondrial | Top1mt |
| 1388650_at | topoisomerase (DNA) 2 alpha | Top2a |
| 1368840_at | TORID | Torid |
| 1378617_at | transcriptional regulating factor 1 (predicted) | Trerf1_predicted |
| 1392188_at | thioredoxin domain containing 9 | Txndc9 |
| 1373037_at | ubiquitin-conjugating enzyme E2L 6 (predicted) | Ube2l6_predicted |
| 1382741_at | ubiquitin protein ligase E3A (predicted) | Ube3a_predicted |
| 1383510_at | ubiquitin protein ligase E3A (predicted) | Ube3a_predicted |
| 1381542_at | UBX domain containing 2 (predicted) | Ubxd2_predicted |
| 1367938_at | UDP-glucose dehydrogenase | Ugdh |
| 1371415_at | ubiquinol-cytochrome c reductase hinge protein (predicted) | Uqcrh_predicted |
| 1367574_at | vimentin | Vim |
| 1368889_at | vesicle transport through interaction with t-SNAREs homolog 1A (yeast) | Vti1a |
| 1369263_at | wingless-type MMTV integration site 5A | Wnt5a |
| 1385343_at | X-box binding protein 1 | Xbp1 |
| 1380071_at | zinc finger CCCH type domain containing 1 (predicted) | Zc3hdc1_predicted |
| 1384452_at | zinc finger, CCHC domain containing 7 (predicted) | Zcchc7_predicted |
| 1394975_at | zinc finger, matrin-like (predicted) | Zfml_predicted |
| 1383053_x_at | zinc finger protein 91 | Zfp91 |
| 1383052_a_at | zinc finger protein 91 | Zfp91 |
| 1376917_at | zinc finger protein 292 | Znf292 |
| 1380025_at | Rattus norvegicus catechol-O-methyltransferase (Comt), mRNA. | Comt |
| 1380240_at | Bone morphogenetic protein 1 | Bmp1 |

Genes and gene fragments identified as changing significantly in animals that were maternally deprived, relative to non-deprived control animals on postnatal day 30 (P30) are provided in Table 7.

TABLE 7

| Fragment Name | Gene Name | Gene Symbol |
|---|---|---|
| 1370913_at | (Best5 protein, hypothetical gene supported by NM_138881) | (Best5, LOC497812) |
| 1370892_at | (complement component 4, gene 2, complement component 4a) | (C4-2, C4a) |
| 1387210_at | (discs, large homolog 4 (Drosophila), hypothetical gene supported by | (Dlgh4, LOC497670) |
| | NM_019621) | |
| 1387995_a_at | (interferon induced transmembrane protein 2 (1-8D), interferon induced | (Ifitm2, Ifitm3) |
| | transmembrane protein 3) | |
| 1372604_at | (hypothetical protein LOC503164, similar to RIKEN cDNA 2210421G13) | (LOC315106, LOC503164) |
| 1382331_at | (similar to RIKEN cDNA 0610038L10 gene, similar to riboflavin kinase) | (LOC317214, LOC499328) |
| 1388212_a_at | (MHC class I RT1.O type 149 processed pseudogene, RT1 class I, T24, | (LOC360231, LOC499402, RT1- |
| | gene 4, RT1 class Ib, locus H2-TL-like (S2), RT1 class Ib, locus S3, similar to | 149, RT1-S2, RT1-S3) |
| | class I histocompatibility antigen alpha chain - cotton-top tamarin) | |
| 1393663_at | (hypothetical gene supported by NM_139339, tramdorin 1) | (LOC497667, Slc36a2) |
| 1373992_at | (similar to MGC108823 protein, similar to interferon-inducible GTPase) | (LOC498872, MGC108823) |
| 1391754_at | (2′,5′-oligoadenylate synthetase 1, 40/46 kDa, 2′-5′ oligoadenylate synthetase | (Oas1, Oas1f, Oas1i) |
| | 1F, 2′-5′ oligoadenylate synthetase 1I) | |
| 1370428_x_at | (RT1 class I, A3, RT1 class I, CE10, RT1 class I, CE3, RT1 class I, CE7, RT1 | (RT1-A2, RT1-A3, RT1-Aw2, |
| | class Ia, locus A2, RT1 class Ib, locus Aw2) | RT1-CE10, RT1-CE3, RT1- |
| | | CE7) |
| 1390706_at | (short form of beta II spectrin, spectrin beta 2) | (Spnb1, Spnb2) |
| 1374780_at | (zinc finger protein 22 (KOX 15), zinc finger protein 422 (predicted)) | (Zfp422_predicted, Znf22) |
| 1380577_at | ATP-binding cassette, sub-family G (WHITE), member 2 | Abcg2 |
| 1398836_s_at | actin, beta | Actb |
| 1369063_at | acidic (leucine-rich) nuclear phosphoprotein 32 family, member A | Anp32a |
| 1385017_at | aquarius (predicted) | Aqr_predicted |
| 1383691_at | activating transcription factor 2 | Atf2 |
| 1389470_at | B-factor, properdin | Bf |
| 1388187_at | calcium/calmodulin-dependent protein kinase II alpha subunit | Camk2a |
| 1371278_at | cell division cycle 34 homolog (S. cerevisiae) (predicted) | Cdc34_predicted |
| 1369239_at | chloride channel 5 | Clcn5 |
| 1387420_at | chloride intracellular channel 4 | Clic4 |
| 1369136_at | Cytochrome P450, subfamily IIA (phenobarbital-inducble)/(Cytochrome P450 | Cyp2a3a |
| | IIA3) | |
| 1371142_at | cytochrome P450, subfamily 2G, polypeptide 1 | Cyp2g1 |
| 1391463_at | DEAD (Asp-Glu-Ala-Asp) box polypeptide 58 (predicted) | Ddx58_predicted |
| 1391602_at | developmentally regulated GTP binding protein 1 (predicted) | Drg1_predicted |
| 1395274_at | dystonin (predicted) | Dst_predicted |
| 1382314_at | interferon, alpha-inducible protein (clone IFI-15K) (predicted) | G1p2_predicted |
| 1368332_at | guanylate nucleotide binding protein 2 | Gbp2 |
| 1369640_at | gap junction membrane channel protein alpha 1 | Gja1 |
| 1387906_a_at | GNAS complex locus | Gnas |
| 1388085_at | glutathione peroxidase 6 | Gpx6 |
| 1381014_at | interferon-induced protein 44 (predicted) | Ifi44_predicted |
| 1384180_at | interferon-induced protein with tetratricopeptide repeats 2 (predicted) | Ifit2_predicted |
| 1379568_at | interferon-induced protein with tetratricopeptide repeats 2 (predicted) | Ifit2_predicted |
| 1383564_at | interferon regulatory factor 7 (predicted) | Irf7_predicted |
| 1383448_at | interferon dependent positive acting transcription factor 3 gamma (predicted) | Isgf3g_predicted |
| 1374627_at | interferon dependent positive acting transcription factor 3 gamma (predicted) | Isgf3g_predicted |
| 1369846_at | involucrin | Ivl |
| 1382535_at | potassium channel tetramerisation domain containing 12 (predicted) | Kctd12_predicted |
| 1394068_x_at | Kruppel-like factor | Klf2 |
| 1390615_at | karyopherin alpha 1 (importin alpha 5) | Kpna1 |
| 1387946_at | lectin, galactoside-binding, soluble, 3 binding protein | Lgals3bp |
| 1387027_a_at | lectin, galactose binding, soluble 9 | Lgals9 |
| 1369149_at | LIM motif-containing protein kinase 1 | Limk1 |
| 1391426_a_at | similar to PEST-containing nuclear protein | LOC288165 |
| 1393436_at | similar to potential ligand-binding protein | LOC309100 |
| 1375539_at | similar to T-Brain-1 | LOC311078 |
| 1389034_at | similar to ubiquitin specific protease UBP43 | LOC312688 |
| 1399073_at | similar to HSPC263 | LOC314660 |
| 1393711_at | similar to epididymal protein | LOC361599 |
| 1381556_at | similar to BC013672 protein | LOC498673 |
| 1385397_at | Ab1-219 | LOC499991 |
| 1376693_at | similar to OEF2 | LOC500011 |
| 1376908_at | similar to This ORF is capable of encoding 404aa which is homologous to | MGC94037 |
| | two human interferon-inducible proteins, 54 kDa and 56 kDa proteins; ORF | |
| 1380037_at | hypothetical LOC292764 | MGC94040 |
| 1383606_at | membrane targeting (tandem) C2 domain containing 1 | Mtac2d1 |
| 1371015_at | myxovirus (influenza virus) resistance 1 | Mx1 |
| 1369202_at | myxovirus (influenza virus) resistance 2 | Mx2 |
| 1387283_at | myxovirus (influenza virus) resistance 2 | Mx2 |
| 1390633_at | cystatin related protein 2 | P22k15 |
| 1368958_at | protein kinase C and casein kinase substrate in neurons 1 | Pacsin1 |
| 1381386_at | processing of precursor 5, ribonuclease P/MRP family (S. cerevisiae) | Pop5_predicted |
| | (predicted) | |
| 1370995_at | POU domain, class 2, transcription factor 1 | Pou2f1 |
| 1387242_at | Protein kinase, interferon-inducible double stranded RNA dependent | Prkr |
| 1378287_at | radixin | Rdx |
| 1375542_at | radixin | Rdx |
| 1376719_at | similar to hypothetical protein D4Ertd89e (predicted) | RGD1305703_predicted |
| 1376789_at | similar to Myosin light chain kinase 2, skeletal/cardiac muscle (MLCK2) | RGD1305801_predicted |
| | (predicted) | |
| 1379285_at | similar to 5830458K16Rik protein (predicted) | RGD1306974_predicted |
| 1377950_at | similar to interferon-inducible GTPase (predicted) | RGD1309362_predicted |
| 1397691_at | similar to KIAA2026 protein (predicted) | RGD1311595_predicted |
| 1387189_at | solute carrier family 22, member 3 | Slc22a3 |
| 1381394_at | PDZ protein Mrt1 | Snx27 |
| 1368835_at | signal transducer and activator of transcription 1 | Stat1 |
| 1387354_at | signal transducer and activator of transcription 1 | Stat1 |
| 1397148_at | stomatin (Epb7.2)-like 3 (predicted) | Stoml3_predicted |
| 1379567_at | thyroid hormone receptor associated protein 1 (predicted) | Thrap1_predicted |
| 1384419_at | thymocyte selection-associated HMG box gene (predicted) | Tox_predicted |
| 1367598_at | transthyretin | Ttr |
| 1368591_at | upstream transcription factor 2 | Usf2 |
| 1386909_a_at | voltage-dependent anion channel 1 | Vdac1 |
| 1389993_at | WD repeat domain 33 (predicted) | Wdr33_predicted |
| 1381034_at | wingless related MMTV integration site 10a (predicted) | Wnt10a_predicted |
| 1380071_at | zinc finger CCCH type domain containing 1 (predicted) | Zc3hdc1_predicted |
| 1385658_at | zinc finger protein 313 | Znf313 |

Genes and gene fragments identified as changing in animals that were maternally deprived, relative to non-deprived control animals on postnatal day 40 (P40) are provided in Table 8.

TABLE 8

| Fragment Name | Gene Name | Gene Symbol |
|---|---|---|
| 1370116_at | septin 3 | 3-Sep |
| 1370913_at | (Best5 protein, hypothetical gene supported by NM_138881) | (Best5, LOC497812) |
| 1368000_at | (complement component 3, hypothetical gene supported by NM_016994) | (C3, LOC497841) |
| 1370892_at | (complement component 4, gene 2, complement component 4a) | (C4-2, C4a) |
| 1381314_at | (cadherin 4, similar to R-cadherin) | (Cdh4, LOC311710) |
| 1370363_at | (carboxylesterase 3, carboxylesterase-like) | (Ces3, LOC291863) |
| 1369984_at | (COX17 homolog, cytochrome c oxidase assembly protein (yeast), popeye | (Cox17, Popdc2) |
| | domain containing 2) | |
| 1395412_at | (GPI-anchored membrane protein 1 (predicted), similar to GPI-anchored | (Gpiap1_predicted, |
| | membrane protein 1) | LOC362174) |
| 1395173_at | (GPI-anchored membrane protein 1 (predicted), similar to GPI-anchored | (Gpiap1_predicted, |
| | membrane protein 1) | LOC362174) |
| 1368319_a_at | (HS1 binding protein, homer homolog 1 (Drosophila)) | (Homer1, Hs1bp1) |
| 1387995_a_at | (interferon induced transmembrane protein 2 (1-8D), interferon induced | (Ifitm2, Ifitm3) |
| | transmembrane protein 3) | |
| 1387458_at | (ring finger protein 4, similar to CG14998-PC, isoform C) | (LOC305453, Rnf4) |
| 1372604_at | (hypothetical protein LOC503164, similar to RIKEN cDNA 2210421G13) | (LOC315106, LOC503164) |
| 1388212_a_at | (MHC class I RT1.O type 149 processed pseudogene, RT1 class I, T24, | (LOC360231, LOC499402, RT1- |
| | gene 4, RT1 class Ib, locus H2-TL-like (S2), RT1 class Ib, locus S3, similar to | 149, RT1-S2, RT1-S3) |
| | class I histocompatibility antigen alpha chain - cotton-top tamarin) | |
| 1389734_x_at | (MHC class I RT1.O type 149 processed pseudogene, RT1 class I, T24, | (LOC360231, LOC499402, RT1- |
| | gene 4, RT1 class Ib, locus H2-TL-like (S2), RT1 class Ib, locus S3, similar to | 149, RT1-S2, RT1-S3) |
| | class I histocompatibility antigen alpha chain - cotton-top tamarin) | |
| 1388164_at | (MHC class I RT1.O type 149 processed pseudogene, RT1 class I, T24, | (LOC360231, LOC499402, RT1- |
| | gene 4, RT1 class Ib, locus H2-TL-like (S2), RT1 class Ib, locus S3, similar to | 149, RT1-S2, RT1-S3) |
| | class I histocompatibility antigen alpha chain - cotton-top tamarin) | |
| 1377174_at | (SET binding factor 1 (predicted), similar to mKIAA3020 protein) | (LOC362985, Sbf1_predicted) |
| 1392655_at | (similar to Nuclear autoantigen Sp-100 (Speckled 100 kDa) (Nuclear dot- | (LOC363269, LOC501175) |
| | associated Sp100 protein), similar to SP140 nuclear body protein isoform 1) | |
| 1369973_at | (hypothetical gene supported by NM_017154, xanthine dehydrogenase) | (LOC497811, Xdh) |
| 1380446_at | (LOC498801, myeloid/lymphoid or mixed-lineage leukemia (trithorax | (LOC498801, Mllt10_predicted) |
| | homolog, Drosophila); translocated to, 10 (predicted)) | |
| 1373992_at | (similar to MGC108823 protein, similar to interferon-inducible GTPase) | (LOC498872, MGC108823) |
| 1393540_at | (myosin heavy chain, polypeptide 6, myosin, heavy polypeptide 7, cardiac | (Myh6, Myh7) |
| | muscle, beta) | |
| 1391754_at | (2′,5′-oligoadenylate synthetase 1, 40/46 kDa, 2′-5′ oligoadenylate synthetase | (Oas1, Oas1f, Oas1i) |
| | 1F, 2′-5′ oligoadenylate synthetase 1I) | |
| 1371152_a_at | (2′,5′-oligoadenylate synthetase 1, 40/46 kDa, 2′-5′ oligoadenylate synthetase | (Oas1, Oas1i, Oas1k) |
| | 1I, 2′-5′ oligoadenylate synthetase 1K) | |
| 1370428_x_at | (RT1 class I, A3, RT1 class I, CE10, RT1 class I, CE3, RT1 class I, CE7, RT1 | (RT1-A2, RT1-A3, RT1-Aw2, |
| | class Ia, locus A2, RT1 class Ib, locus Aw2) | RT1-CE10, RT1-CE3, RT1- |
| | | CE7) |
| 1388071_x_at | (RT1 class I, A3, RT1 class I, CE10, RT1 class I, CE7, RT1 class Ib, locus | (RT1-A3, RT1-Aw2, RT1-CE10, |
| | Aw2) | RT1-CE7) |
| 1370429_at | (RT1 class I, A3, RT1 class I, CE10, RT1 class I, CE7, RT1 class Ib, locus | (RT1-A3, RT1-Aw2, RT1-CE10, |
| | Aw2) | RT1-CE7) |
| 1387555_at | amiloride-sensitive cation channel 2, neuronal | Accn2 |
| 1380533_at | amyloid beta (A4) precursor protein | App |
| 1391658_at | archain | Arcn1 |
| 1381151_at | AT rich interactive domain 4B (Rbp1 like) | Arid4b |
| 1377191_at | ATP synthase, H+ transporting, mitochondrial F0 complex, subunit e | Atp5i |
| 1396279_at | ATPase, H+ transporting, lysosomal accessory protein 2 | Atp6ap2 |
| 1389470_at | B-factor, properdin | Bf |
| 1386774_at | BMP/retinoic acid-inducible neural-specific protein 2 | Brinp2 |
| 1379157_at | calcium channel, voltage-dependent, L type, alpha 1C subunit | Cacna1c |
| 1371687_at | calnexin | Canx |
| 1387178_a_at | cystathionine beta synthase | Cbs |
| 1368887_at | cadherin 22 | Cdh22 |
| 1387420_at | chloride intracellular channel 4 | Clic4 |
| 1370864_at | collagen, type 1, alpha 1 | Col1a1 |
| 1387854_at | procollagen, type I, alpha 2 | Col1a2 |
| 1370155_at | procollagen, type I, alpha 2 | Col1a2 |
| 1370959_at | collagen, type III, alpha 1 | Col3a1 |
| 1387243_at | cytochrome P450, family 1, subfamily a, polypeptide 2 | Cyp1a2 |
| 1369136_at | Cytochrome P450, subfamily IIA (phenobarbital-inducble)/(Cytochrome P450 | Cyp2a3a |
| | IIA3) | |
| 1368608_at | cytochrome P450, family 2, subfamily f, polypeptide 2 | Cyp2f2 |
| 1371142_at | cytochrome P450, subfamily 2G, polypeptide 1 | Cyp2g1 |
| 1370387_at | cytochrome P450, family 3, subfamily a, polypeptide 13 | Cyp3a13 |
| 1390738_at | DAMP-1 protein | Damp1 |
| 1399162_a_at | damage-specific DNA binding protein 1 | Ddb1 |
| 1391463_at | DEAD (Asp-Glu-Ala-Asp) box polypeptide 58 (predicted) | Ddx58_predicted |
| 1394715_at | Dicer1, Dcr-1 homolog (Drosophila) (predicted) | Dicer1_predicted |
| 1380807_at | discs, large homolog 1 (Drosophila) | Dlgh1 |
| 1393756_at | dentin matrix protein 1 | Dmp1 |
| 1388022_a_at | dynamin 1-like | Dnm1l |
| 1395586_at | eukaryotic translation elongation factor 1 alpha 1 | Eef1a1 |
| 1383447_at | ets variant gene 5 (ets-related molecule) (predicted) | Etv5_predicted |
| 1387715_at | extracellular peptidase inhibitor | Expi |
| 1390049_at | four and a half LIM domains 1 | Fhl1 |
| 1377635_at | flavin containing monooxygenase 2 | Fmo2 |
| 1369258_at | fucosyltransferase 9 | Fut9 |
| 1393145_at | follicular lymphoma variant translocation 1 (predicted) | Fvt1_predicted |
| 1382314_at | interferon, alpha-inducible protein (clone IFI-15K) (predicted) | G1p2_predicted |
| 1368332_at | guanylate nucleotide binding protein 2 | Gbp2 |
| 1395370_at | germ cell-less protein | Gcl |
| 1370146_at | glycine receptor, beta subunit | Glrb |
| 1388066_a_at | G protein-coupled receptor kinase 6 | Gprk6 |
| 1388085_at | glutathione peroxidase 6 | Gpx6 |
| 1370952_at | glutathione S-transferase, mu 2 | Gstm2 |
| 1382902_at | potential ubiquitin ligase | Herc6 |
| AFFX_Rat_Hexokinase_M_at | hexokinase 1 | Hk1 |
| 1387994_at | hydroxysteroid (17-beta) dehydrogenase 9 | Hsd17b9 |
| 1374551_at | interferon-induced protein 35 (predicted) | Ifi35_predicted |
| 1381014_at | interferon-induced protein 44 (predicted) | Ifi44_predicted |
| 1384180_at | interferon-induced protein with tetratricopeptide repeats 2 (predicted) | Ifit2_predicted |
| 1379568_at | interferon-induced protein with tetratricopeptide repeats 2 (predicted) | Ifit2_predicted |
| 1383564_at | interferon regulatory factor 7 (predicted) | Irf7_predicted |
| 1376845_at | putative ISG12(b) protein | isg12(b) |
| 1383448_at | interferon dependent positive acting transcription factor 3 gamma (predicted) | Isgf3g_predicted |
| 1374627_at | interferon dependent positive acting transcription factor 3 gamma (predicted) | Isgf3g_predicted |
| 1370773_a_at | Kv channel-interacting protein 2 | Kcnip2 |
| 1387657_at | kinesin family member 3C | Kif3c |
| 1368723_at | linker for activation of T cells | Lat |
| 1387946_at | lectin, galactoside-binding, soluble, 3 binding protein | Lgals3bp |
| 1385601_at | similar to FSHD region gene 1 | LOC304454 |
| 1397512_at | similar to Vps41 protein | LOC306991 |
| 1393436_at | similar to potential ligand-binding protein | LOC309100 |
| 1379748_at | similar to minor histocompatibility antigen precursor | LOC310968 |
| 1389034_at | similar to ubiquitin specific protease UBP43 | LOC312688 |
| 1394459_at | similar to Protein CXorf17 homolog | LOC317423 |
| 1390024_at | similar to osteoclast inhibitory lectin | LOC362447 |
| 1373975_at | similar to thioether S-methyltransferase | LOC368066 |
| 1390506_at | similar to peroxisome proliferator-activated receptor binding protein | LOC497991 |
| 1397230_at | similar to hypothetical protein C130098D09 | LOC498201 |
| 1385213_at | similar to epithelial stromal interaction 1 isoform a | LOC498547 |
| 1383708_at | similar to integrin, beta-like 1 | LOC498564 |
| 1381556_at | similar to BC013672 protein | LOC498673 |
| 1371679_at | similar to Synaptopodin-2 (Myopodin) | LOC499702 |
| 1384547_at | similar to antimicrobial peptide RY2G5 | LOC499925 |
| 1385397_at | Ab1-219 | LOC499991 |
| 1376693_at | similar to OEF2 | LOC500011 |
| 1396099_at | similar to Hypothetical protein MGC30714 | LOC500065 |
| 1382273_at | similar to Apoptosis facilitator Bcl-2-like protein 14 | LOC500348 |
| 1375073_at | similar to sterile alpha motif domain containing 11 | LOC500601 |
| 1398688_at | similar to Spink5 protein | LOC502158 |
| 1375193_at | low density lipoprotein receptor-related protein 11 (predicted) | Lrp11_predicted |
| 1370792_at | microtubule-associated protein, RP/EB family, member 1 | Mapre1 |
| 1379363_at | maltase-glucoamylase (predicted) | Mgam_predicted |
| 1376908_at | similar to This ORF is capable of encoding 404aa which is homologous to | MGC94037 |
| | two human interferon-inducible proteins, 54 kDa and 56 kDa proteins; ORF | |
| 1388204_at | matrix metallopeptidase 13 | Mmp13 |
| 1372683_at | molybdenum cofactor sulfurase (predicted) | Mocos_predicted |
| 1394182_at | myotubularin related protein 4 (predicted) | Mtmr4_predicted |
| 1371015_at | myxovirus (influenza virus) resistance 1 | Mx1 |
| 1369202_at | myxovirus (influenza virus) resistance 2 | Mx2 |
| 1387283_at | myxovirus (influenza virus) resistance 2 | Mx2 |
| 1384255_at | N-ethylmaleimide-sensitive factor attachment protein, gamma (predicted) | Napg_predicted |
| 1383899_at | neural precursor cell expressed, developmentally down-regulated gene 4A | Nedd4a |
| 1395157_at | neural precursor cell expressed, developmentally down-regulated gene 4A | Nedd4a |
| 1395408_at | nitric oxide synthase trafficking | Nostrin |
| 1377497_at | 2′-5′ oligoadenylate synthetase-like 1 (predicted) | Oasl1_predicted |
| 1369835_at | olfactory marker protein | Omp |
| 1394973_at | phosphodiesterase 1C | Pde1c |
| 1372923_at | peroxisomal biogenesis factor 11b (predicted) | Pex11b_predicted |
| 1390423_at | pam, highwire, rpm 1 (predicted) | Phr1_predicted |
| 1369039_at | phosphatidylinositol 4-kinase, catalytic, beta polypeptide | Pik4cb |
| 1371447_at | placenta-specific 8 (predicted) | Plac8_predicted |
| 1387242_at | Protein kinase, interferon-inducible double stranded RNA dependent | Prkr |
| 1376029_at | RAB2, member RAS oncogene family-like | Rab2l |
| 1387641_at | RAB5A, member RAS oncogene family | Rab5a |
| 1371103_at | RAB6B, member RAS oncogene family | Rab6b |
| 1391347_at | RAB8B, member RAS oncogene family | Rab8b |
| 1386900_at | ribosome associated membrane protein 4 | RAMP4 |
| 1396207_at | radixin | Rdx |
| 1390037_at | similar to chromosome 20 open reading frame 58 (predicted) | RGD1305809_predicted |
| 1397861_at | similar to contains transmembrane (TM) region (predicted) | RGD1306235_predicted |
| 1379795_at | similar to RIKEN cDNA 2410002O22 gene (predicted) | RGD1306583_predicted |
| 1390113_a_at | similar to KIAA0731 protein (predicted) | RGD1306683_predicted |
| 1379285_at | similar to 5830458K16Rik protein (predicted) | RGD1306974_predicted |
| 1389849_at | similar to RIKEN cDNA 0610027O18 (predicted) | RGD1307118_predicted |
| 1376144_at | similar to B aggressive lymphoma (predicted) | RGD1307534_predicted |
| 1374337_at | similar to chromosome 17 open reading frame 27 (predicted) | RGD1308168_predicted |
| 1380444_at | similar to RIKEN cDNA 1110017l16 (predicted) | RGD1308977_predicted |
| 1377950_at | similar to interferon-inducible GTPase (predicted) | RGD1309362_predicted |
| 1373262_at | similar to 2310014H01Rik protein (predicted) | RGD1309543_predicted |
| 1385109_at | similar to RIKEN cDNA 4930553M18 (predicted) | RGD1309627_predicted |
| 1373814_at | similar to mKIAA1002 protein (predicted) | RGD1310066_predicted |
| 1372034_at | similar to hypothetical protein MGC29390 (predicted) | RGD1310490_predicted |
| 1372438_at | similar to Nit protein 2 (predicted) | RGD1310494_predicted |
| 1394940_at | similar to hypothetical protein FLJ20037 (predicted) | RGD1311381_predicted |
| 1394695_at | similar to hypothetical protein FLJ20037 (predicted) | RGD1311381_predicted |
| 1379459_at | similar to intracellular membrane-associated calcium-independent | RGD1311444_predicted |
| | phospholipase A2 gamma (predicted) | |
| 1398824_at | coated vesicle membrane protein | Rnp24 |
| 1367661_at | S100 calcium binding protein A6 (calcyclin) | S100a6 |
| 1390777_at | sterol-C5-desaturase (fungal ERG3, delta-5-desaturase) homolog (S. cerevisae) | Sc5d |
| 1384173_at | src family associated phosphoprotein 1 | Scap1 |
| 1368539_at | sodium channel, voltage-gated, type 9, alpha polypeptide | Scn9a |
| 1371063_at | SH3 domain protein 2A | Sh3gl2 |
| 1388064_a_at | solute carrier family 1 (glial high affinity glutamate transporter), member 3 | Slc1a3 |
| 1390825_at | solute carrier family 35, member B3 (predicted) | Slc35b3_predicted |
| 1383970_at | solute carrier family 35, member C2 (predicted) | Slc35c2_predicted |
| 1381922_at | solute carrier family 5 (sodium/glucose cotransporter), member 11 | Slc5a11 |
| 1374391_at | sarcolipin (predicted) | Sln_predicted |
| 1392965_a_at | SPARC related modular calcium binding 2 (predicted) | Smoc2_predicted |
| 1372930_at | SP110 nuclear body protein (predicted) | Sp110_predicted |
| 1368835_at | signal transducer and activator of transcription 1 | Stat1 |
| 1372757_at | signal transducer and activator of transcription 1 | Stat1 |
| 1387876_at | signal transducer and activator of transcription 5B | Stat5b |
| 1397148_at | stomatin (Epb7.2)-like 3 (predicted) | Stoml3_predicted |
| 1369919_at | thyrotroph embryonic factor | Tef |
| 1375951_at | thrombomodulin | Thbd |
| 1374529_at | thrombospondin 2 (predicted) | Thbs2_predicted |
| 1370323_at | thimet oligopeptidase 1 | Thop1 |
| 1371785_at | tumor necrosis factor receptor superfamily, member 12a | Tnfrsf12a |
| 1393423_at | tankyrase, TRF1-interacting ankyrin-related ADP-ribose polymerase 2 | Tnks2_predicted |
| | (predicted) | |
| 1371060_at | tripartite motif protein 23 | Trim23 |
| 1385252_at | tripartite motif protein 34 (predicted) | Trim34_predicted |
| 1387565_at | transient receptor potential cation channel, subfamily V, member 6 | Trpv6 |
| 1391228_at | testis specific gene A2 (predicted) | Tsga2_predicted |
| 1373037_at | ubiquitin-conjugating enzyme E2L 6 (predicted) | Ube2l6_predicted |
| 1385615_at | ubiquitin conjugation factor E4 A | Ube4a |
| 1369850_at | UDP glycosyltransferase 2 family, polypeptide A1 | Ugt2a1 |
| 1369394_at | unc-5 homolog A (C. elegans) | Unc5a |
| 1369597_at | vesicle-associated membrane protein, associated protein B and C | Vapb |
| 1380071_at | zinc finger CCCH type domain containing 1 (predicted) | Zc3hdc1_predicted |
| 1394975_at | zinc finger, matrin-like (predicted) | Zfml_predicted |

Genes and gene fragments identified as changing in animals that were maternally deprived, relative to non-deprived control animals on postnatal day 60 (P60) are provided in Table 9.

TABLE 9

| Fragment Name | Gene Name | Gene Symbol |
|---|---|---|
| 1383096_at | (LOC500962, amyloid beta (A4) precursor-like protein 2) | (Aplp2, LOC500962) |
| 1372977_at | (atlastin-like, cyclin-dependent kinase-like 1 (CDC2-related kinase) | (Cdkl1_predicted, LOC362750, |
| | (predicted), similar to mitogen-activated protein kinase kinase kinase kinase | LOC503027) |
| | 5 isoform 2) | |
| 1387320_a_at | (densin-180, hypothetical gene supported by NM_057142) | (LOC117284, LOC497765) |
| 1384965_at | (LOC499369, serine/threonine kinase 2) | (LOC499369, Slk) |
| 1369063_at | acidic (leucine-rich) nuclear phosphoprotein 32 family, member A | Anp32a |
| 1369752_a_at | calcium/calmodulin-dependent protein kinase IV | Camk4 |
| 1379130_at | CDW92 antigen | Cdw92 |
| 1368366_at | Camello-like 2 | Cml2 |
| 1370991_at | camello-like 3 | Cml3 |
| 1388176_at | camello-like 5 | Cml5 |
| 1389931_at | corticotropin releasing hormone receptor 2 | Crhr2 |
| 1394731_at | casein kinase 1, gamma 3 | Csnk1g3 |
| 1368685_at | chondroitin sulfate proteoglycan 4 | Cspg4 |
| 1381384_at | degenerative spermatocyte homolog (Drosophila) | Degs |
| 1395572_at | DnaJ (Hsp40) homolog, subfamily A, member 4 (predicted) | Dnaja4_predicted |
| 1387306_a_at | early growth response 2 | Egr2 |
| 1369371_a_at | gamma-aminobutyric acid (GABA) B receptor 1 | Gabbr1 |
| 1387935_at | interleukin 3 receptor, alpha chain | Il3ra |
| 1375190_at | potassium channel tetramerisation domain containing 13 | Kctd13 |
| 1376028_at | similar to RIKEN cDNA 4121402D02 | LOC303514 |
| 1399118_at | similar to RIKEN cDNA 4121402D02 | LOC303514 |
| 1394459_at | similar to Protein CXorf17 homolog | LOC317423 |
| 1397334_at | similar to SEC14-like 1 | LOC360668 |
| 1385455_at | similar to sarcoma antigen NY-SAR-27 | LOC499691 |
| 1395472_at | leucine rich repeat containing 17 | Lrrc17 |
| 1393669_at | similar to Ras-related protein Rab-1B | MGC105830 |
| 1387365_at | nuclear receptor subfamily 1, group H, member 3 | Nr1h3 |
| 1391295_at | phosphodiesterase 10A | Pde10a |
| 1372923_at | peroxisomal biogenesis factor 11b (predicted) | Pex11b_predicted |
| 1393673_at | similar to HCDI protein (predicted) | RGD1309307_predicted |
| 1397165_at | SWI/SNF related, matrix associated, actin dependent regulator of | Smarca2 |
| | chromatin, subfamily a, member 2 | |
| 1387852_at | thyroid hormone responsive protein | Thrsp |
| 1395719_at | Tu translation elongation factor, mitochondrial (predicted) | Tufm_predicted |

Genes and gene fragments identified as changing significantly in animals that were maternally deprived, relative to non-deprived control animals on postnatal day 80 (P80) are provided in Table 10.

TABLE 10

| Fragment Name | Gene Name | Gene Symbol |
|---|---|---|
| 1395545_at | (angiotensin II receptor-associated protein, cytochrome P450-like, heat | (Agtrap, Hspa1b, LOC293989, |
| | shock 70 kD protein 1B, proteoglycan peptide core protein, similar to CGI- | MGC95001, Pgsg) |
| | 100-like protein) | |
| 1370892_at | (complement component 4, gene 2, complement component 4a) | (C4-2, C4a) |
| 1387210_at | (discs, large homolog 4 (Drosophila), hypothetical gene supported by | (Dlgh4, LOC497670) |
| | NM_019621) | |
| 1368550_at | (HNF-3/forkhead homolog-1, hypothetical gene supported by NM_022858) | (Foxq1, LOC497713) |
| 1374062_x_at | (G protein-coupled receptor kinase 5, microtubule-associated protein, | (Gprk5, Mapre3) |
| | RP/EB family, member 3) | |
| 1396919_at | (hereditary sensory neuropathy, type II, protein kinase, lysine deficient 1) | (Hsn2, Prkwnk1) |
| 1397130_at | (mitogen activated protein kinase 10, similar to binding protein) | (LOC293702, Mapk10) |
| 1379200_at | (similar to RIKEN cDNA C330027C09 (predicted), similar to Ubiquitin ligase | (LOC303963, |
| | protein DZIP3 (DAZ-interacting protein 3 homolog)) | RGD1310335_predicted) |
| 1389734_x_at | (MHC class I RT1.O type 149 processed pseudogene, RT1 class I, T24, | (LOC360231, LOC499402, RT1- |
| | gene 4, RT1 class Ib, locus H2-TL-like (S2), RT1 class Ib, locus S3, similar | 149, RT1-S2, RT1-S3) |
| | to class I histocompatibility antigen alpha chain - cotton-top tamarin) | |
| 1372751_at | (LOC500936, hypothetical LOC366994) | (LOC366994, LOC500936) |
| 1392177_at | (LOC498458, protein tyrosine phosphatase, receptor type, G) | (LOC498458, Ptprg) |
| 1372640_at | (LOC499410, protease inhibitor 16 (predicted)) | (LOC499410, Pi16_predicted) |
| 1395480_at | (RAD51 homolog (S. cerevisiae) (predicted), similar to AF15q14 protein | (LOC499869, LOC499870, |
| | isoform 2, similar to DNA repair protein RAD51 homolog 1) | Rad51_predicted) |
| 1383356_at | (LOC500443, similar to dynein, axonemal, intermediate chain 1) | (LOC500442, LOC500443) |
| 1398945_at | (similar to RCK, trehalase (brush-border membrane glycoprotein)) | (LOC500988, Treh) |
| 1371017_at | (T-cell receptor gamma chain, similar to TCR V gamma 3) | (LOC502132, Tcrg) |
| 1369138_a_at | (parkin, similar to mature parasite-infected erythrocyte surface antigen like | (LOC502231, Park2) |
| | precursor (2N179)) | |
| 1370428_x_at | (RT1 class I, A3, RT1 class I, CE10, RT1 class I, CE3, RT1 class I, CE7, | (RT1-A2, RT1-A3, RT1-Aw2, |
| | RT1 class Ia, locus A2, RT1 class Ib, locus Aw2) | RT1-CE10, RT1-CE3, RT1- |
| | | CE7) |
| 1368330_at | apoptosis antagonizing transcription factor | Aatf |
| 1370955_at | a disintegrin and metalloprotease domain 10 | Adam10 |
| 1382206_a_at | A kinase (PRKA) anchor protein 2 (predicted) | Akap2_predicted |
| 1388765_at | murine thymoma viral (v-akt) oncogene homolog 2 | Akt2 |
| 1375263_at | activated leukocyte cell adhesion molecule | Alcam |
| 1368776_at | arachidonate 5-lipoxygenase | Alox5 |
| 1396195_at | ankyrin repeat domain 13 (predicted) | Ankrd13_predicted |
| 1369063_at | acidic (leucine-rich) nuclear phosphoprotein 32 family, member A | Anp32a |
| 1398695_at | amyloid beta (A4) precursor protein | App |
| 1387068_at | activity regulated cytoskeletal-associated protein | Arc |
| 1393798_at | alpha thalassemia/mental retardation syndrome X-linked homolog (human) | Atrx |
| 1398004_at | BMP/retinoic acid-inducible neural-specific protein 2 | Brinp2 |
| 1379157_at | calcium channel, voltage-dependent, L type, alpha 1C subunit | Cacna1c |
| 1397151_at | calcium channel, voltage-dependent, alpha 2/delta 3 subunit | Cacna2d3 |
| 1388187_at | calcium/calmodulin-dependent protein kinase II alpha subunit | Camk2a |
| 1387401_at | calsequestrin 2 | Casq2 |
| 1383075_at | cyclin D1 | Ccnd1 |
| 1374540_at | cell division cycle associated 7 (predicted) | Cdca7_predicted |
| 1392140_at | cadherin 11 | Cdh11 |
| 1377402_at | cyclin-dependent kinase (CDC2-like) 10 (predicted) | Cdk10_predicted |
| 1369019_at | cholinergic receptor, nicotinic, alpha polypeptide 5 | Chrna5 |
| 1394008_x_at | ciliary neurotrophic factor receptor | Cntfr |
| 1384227_at | coronin, actin binding protein 1C (predicted) | Coro1c_predicted |
| 1390399_at | cAMP responsive element binding protein-like 2 (predicted) | Crebl2_predicted |
| 1369912_at | v-crk sarcoma virus CT10 oncogene homolog (avian) | Crk |
| 1384339_s_at | casein kinase II, alpha 1 polypeptide | Csnk2a1 |
| 1397217_at | CUG triplet repeat, RNA-binding protein 2 | Cugbp2 |
| 1369068_at | cullin 5 | Cul5 |
| 1379855_at | deleted in colorectal carcinoma | Dcc |
| 1376524_at | hypothetical protein | Dd25 |
| 1391406_at | degenerative spermatocyte homolog (Drosophila) | Degs |
| 1396063_at | DEK oncogene (DNA binding) | Dek |
| 1380807_at | discs, large homolog 1 (Drosophila) | Dlgh1 |
| 1397020_at | discs, large homolog 2 (Drosophila) | Dlgh2 |
| 1375139_at | discs, large homolog 2 (Drosophila) | Dlgh2 |
| 1368146_at | dual specificity phosphatase 1 | Dusp1 |
| 1395586_at | eukaryotic translation elongation factor 1 alpha 1 | Eef1a1 |
| 1369540_at | EF hand calcium binding protein 1 | Efcbp1 |
| 1370542_a_at | E74-like factor 1 | Elf1 |
| 1382710_at | ectodermal-neural cortex 1 | Enc1 |
| 1390054_at | ectodermal-neural cortex 1 | Enc1 |
| 1369453_at | Epsin 1 | Epn1 |
| 1372823_at | family with sequence similarity 36, member A (predicted) | Fam36a_predicted |
| 1392339_at | phenylalanine-tRNA synthetase-like, beta subunit | Farslb |
| 1383516_at | fibrinogen-like 2 | Fgl2 |
| 1390936_at | FXYD domain-containing ion transport regulator 3 | Fxyd3 |
| 1369371_a_at | gamma-aminobutyric acid (GABA) B receptor 1 | Gabbr1 |
| 1392320_s_at | FKBP-associated protein | Glmn |
| 1384819_at | glucagon-like peptide 2 receptor | Glp2r |
| 1398530_at | guanine nucleotide binding protein (G protein), gamma 11 | Gng11 |
| 1394578_at | glutamate receptor, ionotropic, 2 | Gria2 |
| 1393995_at | glutamate receptor, ionotropic, 2 | Gria2 |
| 1392248_at | glutamate receptor, ionotropic, delta 1 | Grid1 |
| 1370267_at | glycogen synthase kinase 3 beta | Gsk3b |
| 1378614_at | Huntington disease gene homolog | Hdh |
| 1395981_at | helicase, ATP binding 1 (predicted) | Helic1_predicted |
| AFFX_Rat_Hexokinase_M_at | hexokinase 1 | Hk1 |
| 1379546_at | hematological and neurological expressed sequence 1 | Hn1 |
| 1392329_at | homeodomain leucine zipper-encoding gene | Homez |
| 1385931_at | hook homolog 3 | Hook3 |
| 1376046_at | hook homolog 3 | Hook3 |
| 1367648_at | insulin-like growth factor binding protein 2 | Igfbp2 |
| 1392246_at | immunoglobulin superfamily, member 4A (predicted) | Igsf4a_predicted |
| 1392467_at | inositol (myo)-1(or 4)-monophosphatase 2 | Impa2 |
| 1396701_at | kalirin, RhoGEF kinase | Kalrn |
| 1369133_a_at | potassium voltage gated channel, Shaw-related subfamily, member 3 | Kcnc3 |
| 1397599_at | potassium voltage-gated channel, subfamily Q, member 3 | Kcnq3 |
| 1386041_a_at | Kruppel-like factor | Klf2 |
| 1387260_at | Kruppel-like factor 4 (gut) | Klf4 |
| 1367880_at | laminin, beta 2 | Lamb2 |
| 1367628_at | lectin, galactose binding, soluble 1 | Lgals1 |
| 1369149_at | LIM motif-containing protein kinase 1 | Limk1 |
| 1370853_at | CaM-kinase II inhibitor alpha | LOC287005 |
| 1392723_at | similar to ankyrin repeat domain protein 17 isoform b | LOC289521 |
| 1385922_at | similar to RIKEN cDNA 5830434P21 | LOC296637 |
| 1382881_at | similar to EXCretory canal abnormal EXC-7, ELAV type RNA binding | LOC298705 |
| | protein (48.7 kD) (exc-7) | |
| 1385713_at | similar to NAKAP95 | LOC299569 |
| 1390375_at | similar to NAKAP95 | LOC299569 |
| 1384799_at | similar to KIAA2022 protein | LOC302396 |
| 1376243_at | similar to serine (or cysteine) proteinase inhibitor, Glade B (ovalbumin), | LOC304692 |
| | member 12 | |
| 1377961_at | similar to AT motif-binding factor | LOC307829 |
| 1375539_at | similar to T-Brain-1 | LOC311078 |
| 1396803_at | similar to THO complex 2 | LOC313308 |
| 1382551_at | similar to Intersectin 2 (SH3 domain-containing protein 1B) (SH3P18) | LOC313934 |
| | (SH3P18-like WASP associated protein) | |
| 1383673_at | similar to Nap1I2 | LOC317247 |
| 1385427_at | similar to RNA-binding protein Musashi2-S | LOC360596 |
| 1389099_at | similar to hypothetical protein MGC51082 | LOC361519 |
| 1399056_at | similar to CG10585-PA | LOC365592 |
| 1386070_at | similar to IRA1 protein | LOC365755 |
| 1374421_at | similar to Williams-Beuren syndrome deletion transcript 9 homolog | LOC368002 |
| 1380728_at | similar to collapsin response mediator protein-2A | LOC498539 |
| 1375821_at | similar to ankyrin repeat domain 26 | LOC498767 |
| 1395081_at | similar to nuclear receptor coactivator 7 | LOC498995 |
| 1384809_at | LOC499283 | LOC499283 |
| 1389986_at | LOC499304 | LOC499304 |
| 1374028_at | similar to CDNA sequence BC024479 | LOC500974 |
| 1385686_at | similar to SPRR1b | LOC502543 |
| 1371405_at | similar to hypothetical protein MGC52110 | LOC503252 |
| 1367984_at | CTD-binding SR-like rA1 | LOC56081 |
| 1368861_a_at | myelin-associated glycoprotein | Mag |
| 1384361_at | mal, T-cell differentiation protein 2 | Mal2 |
| 1379456_at | mitochondrial carrier triple repeat 1 (predicted) | Mcart1_predicted |
| 1386478_at | mitochondrial carrier triple repeat 1 (predicted) | Mcart1_predicted |
| 1385101_a_at | Unknown (protein for MGC: 73017) | MGC73017 |
| 1395223_at | similar to hypothetical protein MGC35097 | MGC94736 |
| 1376853_at | similar to RIKEN cDNA 2310042P20 | MGC94954 |
| 1368279_at | myeloid/lymphoid or mixed-lineage leukemia (trithorax (Drosophila) | Mllt3 |
| | homolog); translocated to, 3 | |
| 1377534_at | mannose receptor, C type 1 (predicted) | Mrc1_predicted |
| 1386114_at | MAS-related G protein-coupled receptor, member B4 | Mrgprb4 |
| 1388853_at | mitochondrial ribosomal protein L54 (predicted) | Mrpl54_predicted |
| 1387786_at | myotrophin | Mtpn |
| 1376648_at | v-myc myelocytomatosis viral related oncogene, neuroblastoma derived | Mycn |
| | (avian) | |
| 1379472_at | NAD synthetase 1 | Nadsyn1 |
| 1393881_at | NMDA receptor-regulated gene 1 (predicted) | Narg1_predicted |
| 1388167_at | nuclear factor I/B | Nfib |
| 1394778_at | nuclear factor I/B | Nfib |
| 1391256_at | N-myristoyltransferase 2 | Nmt2 |
| 1395083_at | neuro-oncological ventral antigen 1 | Nova1 |
| 1374959_at | NAD(P)H dehydrogenase, quinone 2 | Nqo2 |
| 1397004_at | nuclear receptor subfamily 3, group C, member 1 | Nr3c1 |
| 1371069_at | ion transporter protein | Nritp |
| 1391182_at | OTU domain, ubiquitin aldehyde binding 2 (predicted) | Otub2_predicted |
| 1388353_at | proliferation-associated 2G4, 38 kDa | Pa2g4 |
| 1392480_at | poly(A) binding protein, nuclear 1 | Pabpn1 |
| 1368958_at | protein kinase C and casein kinase substrate in neurons 1 | Pacsin1 |
| 1376247_at | phosphoenolpyruvate carboxykinase 2 (mitochondrial) (predicted) | Pck2_predicted |
| 1389396_at | 6-phosphofructo-2-kinase/fructose-2,6-biphosphatase 4 | Pfkfb4 |
| 1371776_at | phosphatidylinositol 3-kinase, regulatory subunit, polypeptide 1 | Pik3r1 |
| 1384558_at | placenta-specific 9 (predicted) | Plac9_predicted |
| 1379690_at | polymerase (DNA-directed), delta interacting protein 3 (predicted) | Poldip3_predicted |
| 1384815_at | protein phosphatase 3, catalytic subunit, alpha isoform | Ppp3ca |
| 1379175_at | protein phosphatase 3, catalytic subunit, alpha isoform | Ppp3ca |
| 1398242_at | protein phosphatase 5, catalytic subunit | Ppp5c |
| 1368240_a_at | protein kinase C, beta 1 | Prkcb1 |
| 1374033_at | proteasome (prosome, macropain) subunit, beta type 10 (predicted) | Psmb10_predicted |
| 1374803_at | proteasome (prosome, macropain) 26S subunit, non-ATPase, 11 | Psmd11_predicted |
| | (predicted) | |
| 1379859_at | proteasome (prosome, macropain) 26S subunit, non-ATPase, 11 | Psmd11_predicted |
| | (predicted) | |
| 1383426_at | proline-serine-threonine phosphatase-interacting protein 1 (predicted) | Pstpip1_predicted |
| 1388430_at | prostate tumor over expressed gene 1 (predicted) | Ptov1_predicted |
| 1384485_at | protein tyrosine phosphatase, receptor type, U | Ptpru |
| 1388363_at | hnRNP-associated with lethal yellow (predicted) | Raly_predicted |
| 1369130_at | RAS guanyl releasing protein 1 | Rasgrp1 |
| 1369129_at | RAS guanyl releasing protein 1 | Rasgrp1 |
| 1376146_at | similar to RIKEN cDNA 2310033P09 (predicted) | RGD1304587_predicted |
| 1374804_at | similar to Autoantigen NGP-1 (predicted) | RGD1305006_predicted |
| 1386088_at | similar to RIKEN cDNA 1200007B05 gene (predicted) | RGD1305072_predicted |
| 1374764_at | similar to 2610033H07Rik protein (predicted) | RGD1305605_predicted |
| 1385862_at | similar to RIKEN cDNA 4930438O05 (predicted) | RGD1305615_predicted |
| 1390037_at | similar to chromosome 20 open reading frame 58 (predicted) | RGD1305809_predicted |
| 1393326_s_at | similar to Pseudoautosomal GTP-binding protein-like protein (predicted) | RGD1305954_predicted |
| 1376263_at | similar to 1810034B16Rik protein (predicted) | RGD1306222_predicted |
| 1393846_at | similar to downregulated in renal cell carcinoma (predicted) | RGD1306327_predicted |
| 1374516_at | similar to RIKEN cDNA 5830457O10 (predicted) | RGD1306894_predicted |
| 1397848_at | similar to RIKEN cDNA 6330406I15 (predicted) | RGD1307396_predicted |
| 1388665_at | similar to RIKEN cDNA 1110020A23 (predicted) | RGD1308134_predicted |
| 1379699_at | similar to FLJ20689 (predicted) | RGD1308907_predicted |
| 1382466_at | similar to RIKEN cDNA 6530403A03 (predicted) | RGD1309020_predicted |
| 1395516_at | similar to hypothetical protein FLJ10154 (predicted) | RGD1310061_predicted |
| 1373814_at | similar to mKIAA1002 protein (predicted) | RGD1310066_predicted |
| 1377648_at | similar to KIAA1838 protein (predicted) | RGD1310304_predicted |
| 1381513_at | similar to RIKEN cDNA 1300017J02 (predicted) | RGD1310507_predicted |
| 1393361_at | similar to chromosome 16 open reading frame 33; minus −99 protein | RGD1310922_predicted |
| | (predicted) | |
| 1376612_at | similar to RIKEN cDNA 1810033A06 (predicted) | RGD1311144_predicted |
| 1373486_at | similar to scotin (predicted) | RGD1312041_predicted |
| 1372639_at | ring finger protein 30 (predicted) | Rnf30_predicted |
| 1379737_a_at | RNA-binding region (RNP1, RRM) containing 2 (predicted) | Rnpc2_predicted |
| 1384654_at | roundabout homolog 1 (Drosophila) | Robo1 |
| 1388413_at | ribosome binding protein 1 homolog 180 kDa (dog) (predicted) | Rrbp1_predicted |
| 1389051_at | retinoid X receptor beta | Rxrb |
| 1395327_at | CD36 antigen (collagen type I receptor, thrombospondin receptor)-like 2 | Scarb2 |
| 1378595_at | NonO/p54nrb homolog | Sfpq |
| 1368109_at | sialyltransferase 9 (CMP-NeuAc:lactosylceramide alpha-2,3- | Siat9 |
| | sialyltransferase) | |
| 1395102_at | potassium channel subunit (Slack) | Slack |
| 1389747_at | solute carrier family 26, member 8 (predicted) | Slc26a8_predicted |
| 1387707_at | solute carrier family 2 (facilitated glucose transporter), member 3 | Slc2a3 |
| 1368046_at | solute carrier family 31 (copper transporters), member 1 | Slc31a1 |
| 1368440_at | solute carrier family 3, member 1 | Slc3a1 |
| 1374391_at | sarcolipin (predicted) | Sln_predicted |
| 1398568_at | synaptosomal-associated protein, 91 kDa homolog (mouse) | Snap91 |
| 1394436_at | sperm associated antigen 9 (predicted) | Spag9_predicted |
| 1377743_at | sprouty protein with EVH-1 domain 1, related sequence (predicted) | Spred1_predicted |
| 1378431_at | serine/arginine-rich protein specific kinase 2 (predicted) | Srpk2_predicted |
| 1398940_at | serine/arginine repetitive matrix 2 (predicted) | Srrm2_predicted |
| 1378886_x_at | Nuclear envelope spectrin repeat protein 1 | Syne1 |
| 1387517_at | synaptotagmin 13 | Syt13 |
| 1397286_at | transcription factor 4 | Tcf4 |
| 1396660_at | transcription factor 4 | Tcf4 |
| 1392382_at | transforming growth factor, beta 2 | Tgfb2 |
| 1381557_at | TGFB inducible early growth response 3 (predicted) | Tieg3_predicted |
| 1374446_at | TCDD-inducible poly(ADP-ribose) polymerase (predicted) | Tiparp_predicted |
| 1394160_at | transmembrane protein 2 (predicted) | Tmem2_predicted |
| 1381822_x_at | TP53 regulating kinase (predicted) | Tp53rk_predicted |
| 1371635_at | transmembrane domain protein regulated in adipocytes | Tpra40 |
| 1371618_s_at | tubulin, beta 3 | Tubb3 |
| 1383510_at | ubiquitin protein ligase E3A (predicted) | Ube3a_predicted |
| 1396477_at | unr protein | Unr |
| 1376256_at | WD repeat and FYVE domain containing 1 (predicted) | Wdfy1_predicted |
| 1369263_at | wingless-type MMTV integration site 5A | Wnt5a |
| 1385343_at | X-box binding protein 1 | Xbp1 |
| 1387129_at | X-ray repair complementing defective repair in Chinese hamster cells 1 | Xrcc1 |
| 1384452_at | zinc finger, CCHC domain containing 7 (predicted) | Zcchc7_predicted |
| 1393795_at | zinc finger homeobox 1b (predicted) | Zfhx1b_predicted |
| 1380529_at | zinc finger protein 207 (predicted) | Zfp207_predicted |
| 1379974_at | zinc finger protein 533 (predicted) | Zfp533_predicted |
| 1377105_at | zinc finger protein 91 | Zfp91 |
| 1397356_at | zinc finger protein 408 (predicted) | Znf408_predicted |
| 1380030_at | zinc finger protein 593 (predicted) | Znf593_predicted |

### Example 4

### Lis-1 mRNA Expression Profiles in PFC in Socially Isolated Animals and PCR Confirmation

This example provides the temporal mRNA expression of Lis-1, a gene that plays a role in signaling cascades involved in schizophrenia in isolation reared animals, as compared to socially reared animals. Isolation reared animals and socially reared controls were maintained as described in Example 1. The microarray was carried out as described in Example 3. Confirmation of the microarray data, using quantitative real-time PCR is described below.

Quantitative Real-Time PCR

Real-time PCR was carried out using TaqMan technology on an ABI Prism 7900HT Sequence Detection System (PE Applied Biosystems, UK). cDNAs, from 1 μg of DNase treated RNA from each animal (n=6 per group) were produced using SuperScript II RNase H Reverse Transcriptase Kit (Invitrogen) and 50-250 ng random primers (Invitrogen). cDNA (0.8 μl) from each sample was amplified using TaqMan® Gene Expression Assay primers and probe (Applied Biosystems, UK), Assay ID Rn_{—}00578324_ml. Relative quantitation was determined by constructing a standard curve for each primer and probe set, using pooled DNA from all the samples. A ribosomal RNA control primer and probe set (Applied Biosystems) was used for normalization purposes.

Preparation of RNA Probes

Riboprobes were purchased for Lis-1 (Applied Biosystems, UK). cDNA (0.8 μl) from each sample was amplified using TaqMan® Gene Expression Assay primers and probe (Applied Biosystems, UK) designed to the Lis-1 gene (GenBank database accession number NM_{—}031763). Relative quantitation was determined by constructing a standard curve for each primer and probe set, using pooled DNA from all the samples. A ribosomal RNA control primer and probe set (Applied Biosystems) was used for normalization purposes.

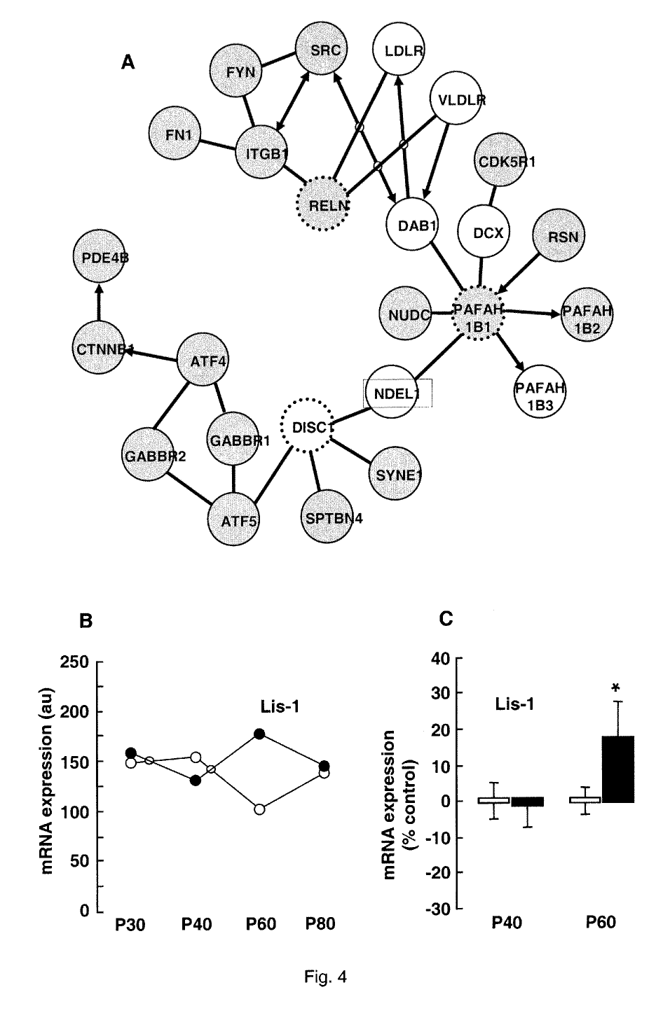

FIG. 4A illustrates an Ingenuity® map demonstrating how Lis-1 is functionally connected to the schizophrenia susceptibility genes DISC1 and RELN. Many other genes in this cluster show dysregulation at the transcriptional level in the isolation rearing model. Lis-1 showed a P60 spike in mRNA expression in isolation reared animals, compared to socially reared animals (FIG. 4B), as with the genes analyzed in Example 3. The temporal pattern of mRNA expression of Lis-1 was confirmed at the time points of interest following rearing, showing an increase at P60 (FIG. 4C).

### Example 5

### mRNA Expression Profiles of Schizophrenia Signaling Genes in PFC in Socially Isolated Animals and PCR Confirmation

This example provides the temporal mRNA expression of genes that implicate signal cascades that play a central role in schizophrenia, including GABAergic receptors (GABAA receptor alpha4 and complexin I) and synaptic structure (synapsin II) in isolation reared animals, as compared to socially reared animals. Isolation reared animals and socially reared controls were maintained as described in Example 1. The microarrary was carried out as described in Example 3. Confirmation of the microarray data, using quantitative real-time PCR was carried out as described in Example 4.

Preparation of RNA Probes

Riboprobes were purchased for GABAA receptor alpha4, complexin I, and synapsin II. cDNA (0.8 μl) from each sample was amplified using these TaqMan® Gene Expression Assay primers and probe (Applied Biosystems, UK) designed to the GABAA receptor alpha4, complexin I, and synapsin II genes (GenBank database accession numbers NM_{—}080587, NM_{—}022864 and NM_{—}019159). Relative quantitation was determined by constructing a standard curve for each primer and probe set, using pooled DNA from all the samples. A ribosomal RNA control primer and probe set (Applied Biosystems) was used for normalization purposes.

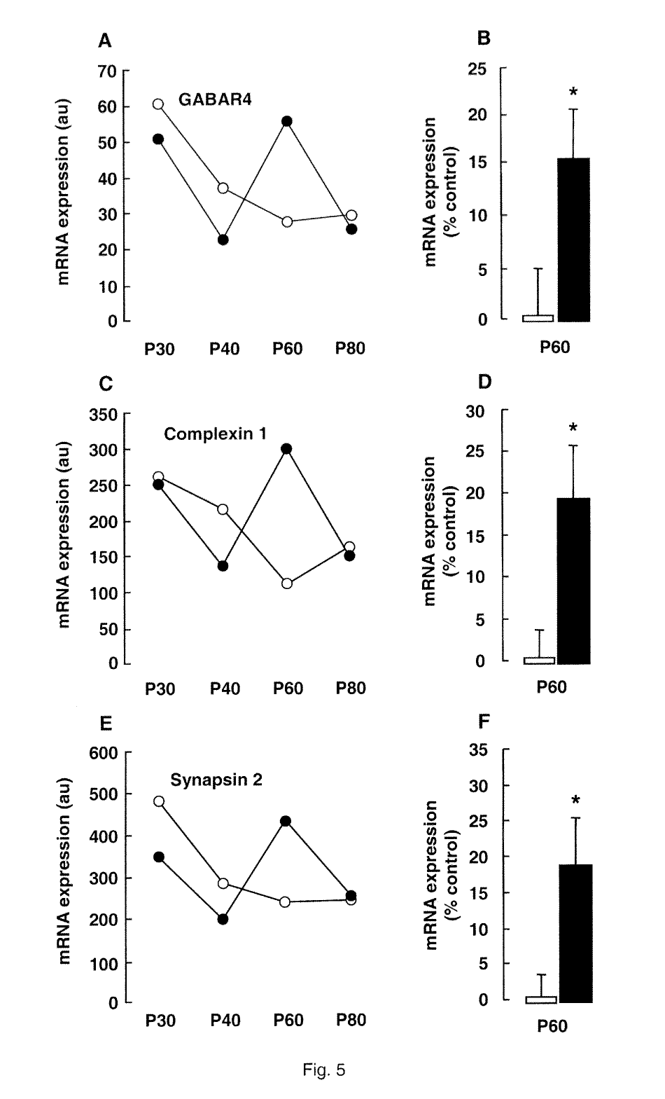

The genes GABAA receptor α4, complexin I, and synapsin II showed a P60 spike in mRNA expression in isolation reared animals, compared to socially reared animals (FIGS. 5A, 5C, and 5E), as with the genes analyzed in Examples 3 and 4. The temporal pattern of mRNA expression of these three genes were confirmed at P60 following rearing, validating the P60 spike (FIGS. 5B, 5D, and 5E).

### Example 6

### mRNA Expression Profiles of Interferon-Regulated Genes in PFC in Socially Isolated and Maternally Deprived Animals

This example provides the temporal mRNA expression of interferon-regulated genes (Interferon-induced protein with tetratricopeptide repeats 2, Interferon regulatory factor 7, and PKR) in isolation reared animals, as compared to maternally-deprived animals, and socially reared animals. Isolation reared animals, maternally deprived animals, and socially reared controls were maintained as described in Example 1. The microarray was carried out as in Example 3. The maternal deprivation model is described below.

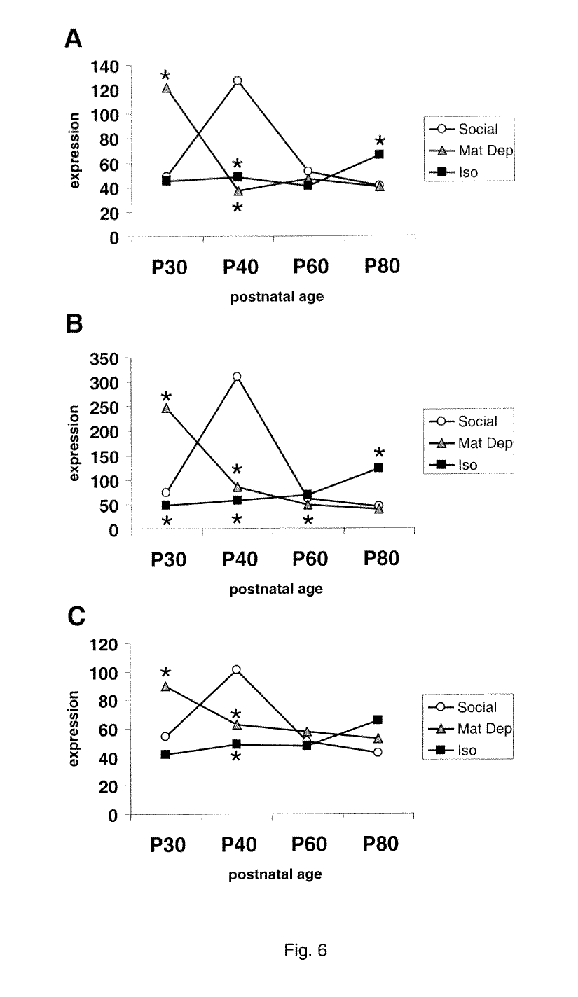

Interferon-regulated genes exhibited a substantial increase in normal social control animals at P40 (FIG. 6). This was absent from both the isolation reared and maternally deprived animals (FIG. 6). The P40 time point is prior to the time when symptoms of the isolation rearing model are present.

### Example 7

### Influence of Prior Environmental Manipulation on Basal Neurotransmitter Levels in the Medial PFC

This example provides basal levels of dopamine, glutamate and GABA in the medial prefrontal cortex of groups of mature Wistar rats that had either been isolation reared, maternally deprived, or were social controls. Isolation rearing was carried out as described in Example 1, and maternal deprivation was carried out as described in Example 6.

Materials and Methods

Brain Microdialysis

Male Wistar rats were anaesthetized on P80 using a Univentor 400 anaesthesia unit (Univentor, Malta) under isoflurane inhalation (4% in air delivered at 6.7 ml/min, with an airflow of 500 ml/min) until the pedal withdrawal reflex was lost. Once anaesthetised the rat was placed in a Kopf stereotaxic frame (David Kopf Instruments, USA) and stabilised with blunt ear bars. Anaesthesia was maintained by continuous administration of isoflurane (2%, delivered at 3.4 ml/min, air flow 500 ml/min). The frontal skull bone was exposed by a saggital incision of the scalp at the midline. A burr hole of 0.8 mm diameter was drilled through the left side of the skull and the dura was carefully incised. A microdialysis probe of concentric design (CMA/12, Carnegie Medicin AB, Solna, Sweden) outer diameter 0.5 mm and a 4 mm length of dialysing membrane was carefully implanted in the medial prefrontal cortex (mPFC). The stereotaxic co-ordinates for the probe in the mPFC (mm from Bregma) were anteroposterior +2.7, mediolateral ±1.4, dorsoventral −6.5 (mm from bone) at a 12□ angle. The incisor bar was set at −3.3 mm (Paxinos and Watson 1998).

During surgery the body temperature was continuously maintained at 37° C. by means of a thermostatically regulated heating pad (CMA 150, Carnegie Medicin AB, Sweden). The flow rate (2 μl/min) of the perfusion medium (sterile Ringer solution, Baxter, UK; formula per 1000 ml: sodium chloride 8.6 g; potassium chloride 300 mg; calcium chloride 300 mg; pH˜6) was maintained constant by a microperfusion pump (CMA 100; Carnegie Medicin AB, Sweden) during implantation on P80 and also for the duration of the microdialysis experiment on P82. The probe was fixed to the skull with stainless steel screws and metacrylic cement (Svedia, Enkoping, Sweden).

On the day of the microdialysis experiment on P82 dialysate samples were collected in 20 min (40 μl) aliquots for separation using high performance liquid chromatography followed by fluorimetric (glutamate) or electrochemical (GABA) detection (Kehr et al., 1989, Morari et al, 1994). A new microdialysis probe was used for each rat. At the end of each experiment animals were killed using a CO2 overdose and the placement of the probes was verified by microscopic examination using a Leitz Cyrostat (Leitz, Germany) or by cresyl violet staining of 30 μM slices cut with a microtome. Animals with incorrect probe position were not included in this study. Data are reported as a percent change from basal values, which were calculated as the mean of the dialysate levels from 3 stable basal samples. All data are expressed as the Mean±SEM. ANOVA with repeated measures and Dunnet's post hoc test were used to test for significant differences between groups (P<0.05).

The neurotransmitter glutamate was determined by precolumn derivatization of a 10 μL dialysate sample with o-phtaldialdehyde/mercaptoethanol reagent and separation by reversed-phase HPLC on a Biophase ODS 5 μM particle column (Knauer, Berlin, Germany). The mobile phase contained 0.1 M sodium acetate, 6.25% methanol, 1.5% tetrahydrofurane, pH 6.95 and was perfused at a flow rate of 1 ml/min A linear gradient system was used to clean the column after elution of glutamate. This involved switching to 100% methanol for 2 mins before switching back to the original acetate buffer. The excitation wavelength in the fluorescence detector (CMA/280, Solna, Sweden) was set at 370 nm and the emission cut off filter was set at 450 nm. The limit of detection was 0.5 pmol/sample for glutamate (Moran et al 1994).

The GABA assay was based on precolumn derivatization of a 10 μl sample with o-phtaldialdehyde/t-butylthiol reagent and separation by reverse-phase HPLC on a Nucleosil 3 C18 column perfused under isocratic conditions at the flow rate of 0.8 mL/min. The mobile phase was 0.15 M sodium acetate, 1 mM EDTA, 50% acetonitrile, pH 5.4. The BAS LC4B electrochemical detector (Bioanalytical Systems, West Lafayette, 1N, USA) was set at +0.75V. The limit of detection was 20 fmol/sample (Kehr et al 1989).

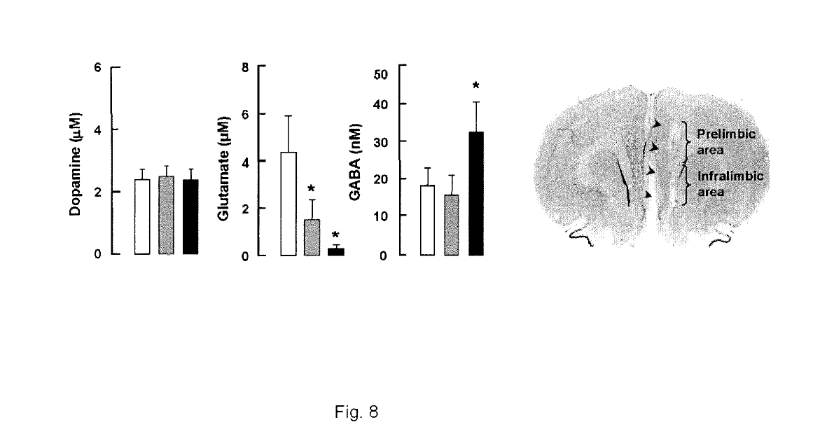

Isolation reared animals exhibited a reduction in mPFC glutamate relative to social controls, as did maternally deprived animals. Maternally deprived animals exhibited an increase in mPFC GABA relative to social controls and isolation reared animals (FIG. 8). There were no differences across groups with respect to dopamine in the mPFC (FIG. 8).

### Example 8

### Influence of Prior Environmental Manipulation on Parvalbumen Cell Density in the Medial PFC

This example provides parvalbumen cell density in the medial PFC of groups of mature Wistar rats that had either been isolation reared, maternally deprived, or were social controls. Isolation rearing was carried out as described in Example 1, and maternal deprivation was carried out as described in Example 6.

Materials and Methods

Parvalbumin Immunohistochemistry

Male Wistar rats were anaesthetized on P80 and perfused with 4% paraformaldehyde (pH 7.4) and brains were removed and stored in the same fixative overnight. Following sacrifice, the whole rat brain was immediately dissected, coated in Optimum Cutting Temperature (OCT) compound to provide an even freezing rate, and lowered into a Cryoprep freezing apparatus (Algen Inc.) containing dry-ice cooled n-hexane. The brains were then stored at −80° C. for later analysis. Sections (12 μm thick) of the prefrontal cortex were taken at level 3.2 mm rostral to bregma using a MICROM Series 550 cryostat at −12° C. on the day of the experiment and were not stored frozen. Sections were thaw mounted onto poly-1-lysine coated slides and immersion fixed for 30 minutes in 70% ethanol followed, by two 10-minute washes in 0.1M PBS (pH 7.3). The sections were then incubated with 80-100 μl of the mouse monoclonal anti-parvalbumin antibody (Swant, CH; Cat No: 235) diluted 1 in 250 with PBS containing 1% BSA and 1% NGS for 20 hours in a humidified chamber at room temperature. Following two 10 minute washes in 0.1M PBS (pH 7.3), the sections were incubated with the secondary antibody, anti mouse IgG FITC (Calbiochem, UK; Cat No. 401244) diluted 1 in 250 in PBS containing 1% BSA and 1% NGS, for 3 h. The section were counterstained by dipping the sections for 2-3 secs in a solution of Hoechst 33258 nuclear stain (Molecular Probes, US; Cat No. H3569) at a dilution of 1 in 2,000 in PBS followed by a 10 minute wash in 0.1M PBS. The sections were mounted in Vectashield and protected with a coverslip. Sections used to quantify immunopositive cells were not counterstained.

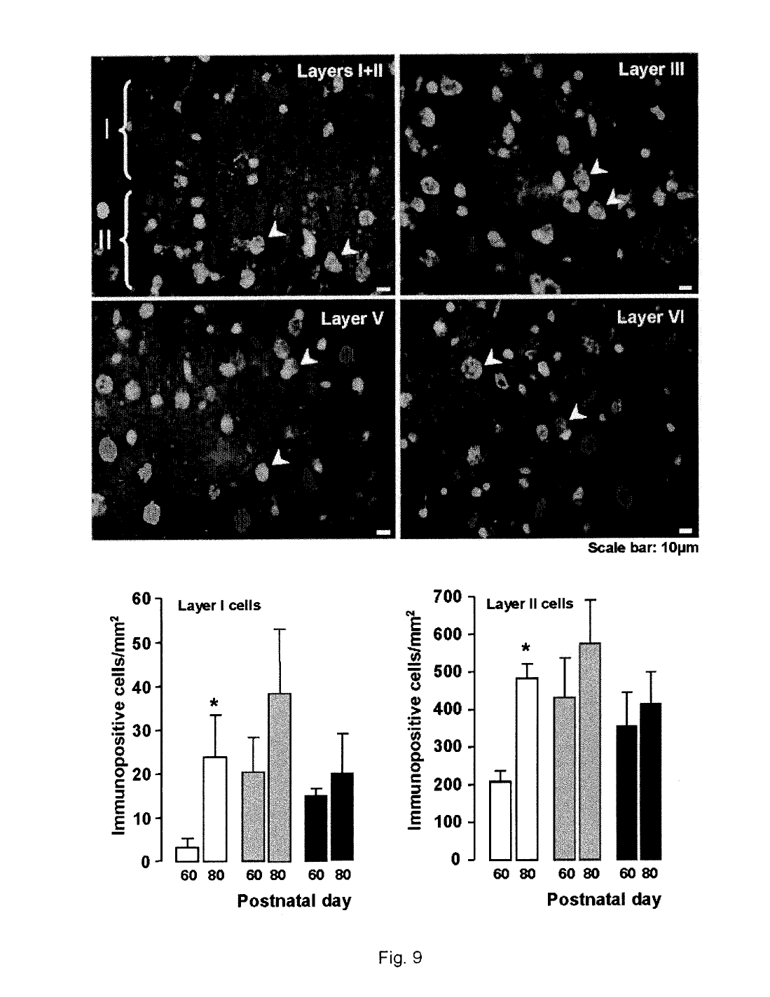

A montage of four separate images representative of the layers of the prelimbic cortex was created (FIG. 9) using a Leica DMLB fluorescence microscope 20× objective. A counting frame (0.898 mm×0.349 mm) with the width of each layer was overlaid on the montage to facilitate counting the layer-specific cell number. Seven separate montages, derived from serial sections obtained from each animal were used to estimate immunopositive cell number and then normalized to cells/mm²/unit area by dividing by the area of each layer.

Socially reared control animals exhibited an increase in parvalbumen cell density in the medial PFC on P80, relative to P60 in Layer I cells. In Layer II cells, socially reared control animals exhibited an increase in parvalbumen cell density in the medial PFC on P80, relative to P60. In both isolation reared animals and maternally deprived animals, no such change in cell density was observed indicating a substantial dysregulation in the development of GABAergic interneurons (FIG. 9).

### Example 9

### Influence of Prior Environmental Manipulation on Synapse Density in the Medial PFC

This example provides synapse density in the medial PFC of groups of mature Wistar rats that had either been isolation reared, maternally deprived, or were social controls. Isolation rearing was carried out as described in Example 1, and maternal deprivation was carried out as described in Example 6.

Materials and Methods

Ultrastructural Analysis

Tissue Processing

Following transcardial perfusion with a 4% (w/v) paraformaldehyde/2% (w/v) glutaraldehyde solution at pH 7.4, brains were removed from the rats and kept overnight in the same fixative. Following post-fixation, the brains were removed from the storage and the cerebellum was detached. The remaining block was fixed to the stage of a vibroslicer (Campden Instruments), with the rostral face uppermost. Sections of 100 μm were collected at a point 3.2 mm rostral to bregma and placed into phosphate buffer (0.1 M) in preparation for processing into epoxy resin. Inter-animal consistency was maintained by reference to bregma as described in a rat brain atlas (Paxinos & Watson, 2005). Subsequent to 30 min post-fixation in 0.1% (w/v) osmium tetroxide (Sigma Chemical Co. Ltd, U.K.), slices were dehydrated and flat embedded with epoxy resin (Agar 100; Agar Scientific, U.K.) by routine methods. Polymerization of the resin permitted the prefrontal cortex to be excised from brain slices and re-embedded in resin filled capsules. Using an ultramicrotome, the re-embedded cortex was identified and semithin sections were taken. These sections were stained with 1% Toluidine Blue (Sigma Chemical Co. Ltd, U.K.) to confirm stereotaxic co-ordinates as 3.2 mm rostral to bregma. Subsequently, serial ultrathin sections of silver/gold interference colour (80 nm thick; Peachey, 1958) were cut from the block face and collected in pairs on electron-lucent coated slot grids (2×1 mm; Agar Scientific, U.K.) Ultrathin sections were counter-stained using uranyl acetate (5% w/v distilled water) and lead citrate (0.3% w/v in 0.1 M sodium hydroxide). Sections were examined in a Tecnai G2 Spirit BioTWIN electron microscope at an accelerating voltage of 120 kV. Images were recorded using a Megaview III CCD, analysed using the analySIS® programme (Soft Imaging Systems) and stored on CD for further analysis.

Stereology

Quantification of ultrastructural features employed an unbiased stereological counting technique, termed the double disector. This method of quantification does not require any assumptions about the size or shape of the object under investigation (Sterio, 1984). The sections containing the objects to be quantified were placed in pairs on slot grids and examined under the electron microscope. Corresponding areas from each section were photographed. A counting frame, drawn in the computer application programme Adobe Photoshop 7®, was placed on the area under investigation on the images obtained from adjacent serial sections. This ‘Gunderson frame’ (Gunderson, 1977) was designed to have two adjoining dotted lines and two adjoining solid lines. At the magnification used for synaptic density quantification (20,500×), the ‘Gunderson frame’ included an area of 13.3138 μm2. One of the pairs of section was randomly, but consistently, selected as the ‘look-up’ and the other as the ‘reference’ section. The number of objects counted in the ‘reference’ image but absent in the ‘look-up’ image was defined as the Q value. In addition, specific counting rules to eliminate bias were followed. These include counting only objects found within and touching the top and right dotted edges of the Gunderson frame. Also, specific definitions for the identification of a synapse were followed. A synapse was only counted if it contained 3 or more vesicles in the pre-synaptic element and a post-synaptic density. For each animal, several disector pairs were analysed until a progressive mean test of Q values consistently showed the accumulative mean value to deviate by less than 10%, as has been recommended previously (Williams, 1977). Each of the layers from I to VI of the cortex were examined comprehensively using these methods. The region of the cortex that was examined was defined using stereotaxic co-ordinates by reference to a rat brain atlas (Paxinos & Watson, 1998) and the individual layers were defined according to known criteria for each layer. As an example, layer I is a relatively cell sparse layer, and it can easily be distinguished from the more densely packed layer II. Layer V contains predomninantly pyramidal shaped soma that runs perpendicular to the pial surface in contrast to the cells of layer VI, which run parallel to the pial surface.

The density of synapses was then estimated using the following equation:

Nv=Q/h·a(fra)

Where Nv=density of objects (synapses) per unit volume; Q=(No. objects in ‘reference’ section)−(No. objects in ‘look-up’ section); h=thickness of the section; a(fra)=area of Gunderson counting frame. All tissue collected for ultrastructural, and immunohistochemical, studies were evaluated by an observer who was ‘blind’ to the experimental conditions and the samples were ‘unblinded’ only when all experiments were complete.

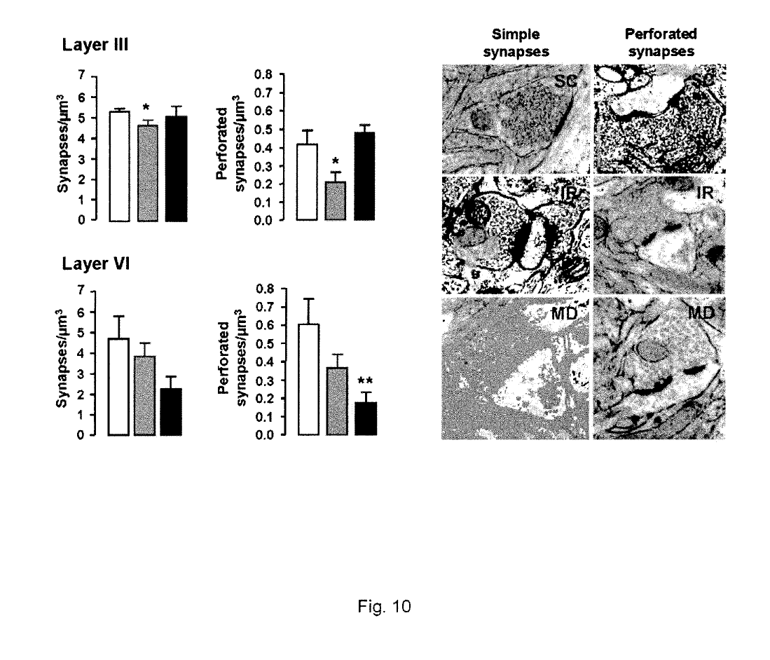

Isolation reared animals exhibited a significant reduction in synaptic density (both synapses and perforated synapses) in the medial PFC, relative to both socially reared controls and maternally deprived animals, in Layer III. Maternally deprived animals exhibited a reduction in perforated synapse density in Layer VI relative to socially reared controls and isolation reared animals (FIG. 10).

### Example 10

### The Deficit in Prepulse Inhibition is Reversed by ICV Midkine Administration

Prepulse inhibition of startle, as described in Example 1, is used as a readout of sensorimotor processing in the prefrontal cortex, and is closely tied with working memory. Midkine administration into the 3rd cerebral ventricle of the brain reversed the deficit observed in prepulse inhibition of startle in animals that were reared in isolation. Isolation rearing and prepulse inhibition of startle were performed as described in Example 1. The experimental design of this study dictated surgical implantation of a guide cannula, following which animals were housed in soft bottom cages floored with sawdust, and a daily intracerbroventricular (i.c.v.) of compounds. However in order to maximize the effect of isolation rearing, surgery was carried out on P80 and i.c.v. injections were carried out as quickly and efficiently as possible.

Animals received an i.c.v. injection of 5 μl of either midkine 0.4 μg/μl or sterile H2O for 3 days prior to and on the day of behavioral testing.

Isolation reared animals exhibited a significant deficit in prepulse inhibition (PPI) of startle. Four daily ICV injections of midkine (2 μg/day) reversed PPI behavior back to normal compared to vehicle-treated animals (Two-way ANOVA, p<0.05 for treatment) (FIG. 11).

1. A method of identifying gene targets associated with schizophrenia or schizophrenia symptoms, comprising:
(a) initiating an animal model of schizophrenia in living animals;
(b) assessing transcriptional regulation in tissue over time in animals that are a model of schizophrenia, wherein the tissue is sampled one or more times after the initiation of the model and optionally one or more times prior to the initiation of the model;
(c) comparing the transcriptional regulation from prior to initiation of the model with transcriptional regulation from after the initiation of the model, and/or with transcriptional regulation assessed from tissue in living animals not subject to a schizophrenia model; and
(d) detecting a transcript that is dysregulated in tissue from animals that are a model of schizophrenia.

2. The method of claim 1, wherein the model of schizophrenia is isolation rearing.

3. The method of claim 1, wherein the model of schizophrenia is maternal deprivation.

4. The method of claim 2, wherein the transcript is dysregulated at a time point selected from the group consisting of postnatal day 30, postnatal day 40, postnatal day 60, and postnatal day 80.

5. The method of claim 1, further comprising comparing the dysregulated transcript with at least one gene previously associated with schizophrenia.

6. The method of claim 1, wherein the dysregulated transcript is selected from the group consisting of SEQ ID NO:1 through SEQ ID NO:16.

7. The method of claim 1, wherein the dysregulated transcript is selected from the group consisting of interferon-induced protein, interferon regulatory factor 7, and PKR.

8. An isolated nucleic acid molecule comprising at least one transcript identified from the method of claim 1.

9. A method of predicting susceptibility to schizophrenia in an individual comprising detecting dyregulation of one or more pre-symptomatic genes or the products thereof from a biological sample provided by the individual, wherein the dysregulation is correlated with an increased likelihood of developing schizophrenia or the symptoms thereof.

10. The method of claim 9, wherein the dysregulation comprises upregulation.

11. The method of claim 9, wherein the individual is asymptomatic for schizophrenia.

12. The method of claim 9, wherein the individual exhibits symptoms of schizophrenia.

13. The method of claim 9, wherein the individual has not been diagnosed with schizophrenia.

14. The method of claim 9, wherein the individual presents one or more risk factors associated with schizophrenia.

15. The method of claim 9, wherein the biological sample is blood.

16. A method of diagnosing schizophrenia in an individual comprising detecting dysregulation of one or more symptomatic genes or the products thereof from a biological sample provided by the individual.

17. The method of claim 16, wherein the dysregulation is correlated with additional factors diagnostic of schizophrenia.

18. The method of claim 16, wherein the one or more genes is selected from the group consisting of SEQ ID NO:1 through SEQ ID NO:16.

19. The method of claim 16, wherein the one or more genes is selected from Tables 3, 4, 7, or 8.

20. A method of preventing the onset of schizophrenia or its symptoms in an individual in need thereof, comprising modulating the expression of a pre-symptomatic gene or products thereof.

21. The method of claim 20, further comprising administering at least one additional method of prevention.

22. A method of treating schizophrenia or its symptoms in an individual in need thereof, comprising modulating a symptomatic gene or expression thereof.

23. The method of claim 22, further comprising administering at least one additional treatment.

24. The method of claim 22, wherein the gene is selected from the group consisting of SEQ ID NO:1 through SEQ ID NO:16.

25. The method of claim 20, further comprising one or more additional schizophrenia treatments.

26. A method of treating schizophrenia or its symptoms comprising administering midkine to an individual in need thereof.

27. A method of treating cognitive deficits comprising administering midkine to an individual in need thereof.


SEQUENCE LISTING (16 sequences)

SEQ ID NO: 1 - DNA, 1368 bp, Rattus norvegicus
ggaaaaggcggcgggccggcggagaggctgtgggtaaagcttcgcggggtttgcggagcagcctcttccccgggatggggctgctggagctgtgcgagcaggtgtttggcaccgccgacctctaccaggtgctgggcgtgcggcgcgaggcttccgacggcgaagtccgacgcggctaccacaaggtgtccctgcaagtgcaccccgacagagtaaaggaggaccagaaagaggacgccacccgccgcttccagatcttggggagagtgtacgcggttctgagtgacaaggagcagaaagcagtgtacgatgaacaggggacagtggacgaagactctgctggcctccaccaagaccgagactgggatgcgtattggagattactctttaaaaagatatcgctagaagatatccaagcttttgaaaagacatacaaaggctccgaagaagagctaaacgatattaagcaagcctatctggacttcaagggcgacatggatcagatcatggagtctgtgctttgtgtacaatacacagatgaacccaggataagaaacatcattcaaaaagctattgatgccaaagaggttccgtcctacaatgccttcgttaaagagtctaaacagaagatgaacgcaaggaaaagaagggctcaggaagaagccaaagaagcagagctgagcaggaaggagctggggctggaaggaggagtggatagcctgaaagcgctcatccagagcagacagaaggatcggcaaaaggaaatggacagttttctggctcagatggaggcaaaatactgcaaaccttccaaaggagggaagagagcagcactcaggaaggagaagaagtaatggagtttcctcttcacgggccctatgtcagtgtacggtgtacacaaggtaaaggcagccactaatgtaacctttgagagaagttgtttgtcctgcctaagccaagcaggattcatcaatctggtcttagtagtttctagaagtcacctaacactgtgtgagggcttcgtgtgaagggttgtggtgtgatggttgggagggtgggtttgacatttgcttccgtggaccttgtgcccaaagagctctatctagaatacaggtttctgtggactgttcttactgatgtgcgtgtaatgaagagattcctatggagaacaccctcagaacacttgctgaacaactgtcctctaaataaatgccagtgtgctctggtcttaatcctacacagatcttcttttctggttctactgttagggtttgtcttctgtccttgcagtagcagacatgtgtattcatattcttgctaactttttttgtaagattgtatatggctaaataaagaaacaatgacaaa

SEQ ID NO: 2 - DNA, 929 bp, Rattus norvegicus
gtcccacgcaggtctttccataggctctttccaacccaccacattggtgactagttttcaagcatcattccaagggaaggcctaagaagggaagcatcttcatgtcgtgcactcagtgcaggtgcgctggtgacaagatgacacgagagaggagaagagtggcgtccgggcttgcctcccaagatgcacttcacctacattgtatctgcagctttctccccacccttccgggtgctccccaaggaggttgcatgcagccaggcagccccagaggatgttttctcagacctcctgagggccacaggtgagtggccgtgtatagtgacgcccgacccatcctcactactgcaatcccagaactgcagggcttggcattttgactctggaagcagagagcagaggggctttggtttttacaacacggactgtcacccgggaaacagatgtgacagagcttttcttctttctcagtggatcaaggtatccttgtgtgtgtcaaagcacaaaccgcatgcacactcacttccgggccacagaaactgggtccgttacattttttggcctttttttcttttacagagtcaacgtgaatgaagaatgtttatcactgtaaaattcaggcggctctggacagtattatatgtacgtattgaccctgattcaagttagccactgagagatcggcgacttatccctcctgttacagaagttgaagaatgtggcccgatgtatattaatcccgttgaagcccaaatcctggtggaagggccttaaaactgggcttggatctcgggcacaccttgacagttggtgttagaaagatactaccttggagctggagcggccaagcggcctagttcaccttatgcttaaggcatctctgggtctacagggaggggtgcctgggccaagtccactcgttaagcagc

SEQ ID NO: 3 - DNA, 1368 bp, Rattus norvegicus; identical to SEQ ID NO: 1

SEQ ID NO: 4 - DNA, 1556 bp, Rattus norvegicus
acaaagtgaggccaaaagcccgggagagcccaggccgagtctaagagcaaacacagaaaacggtctgaaccccgctcttagccctagattcccggtgggctttctggggtttctctggtacccagggtgcctggttgctaggagaccatgcttaggaagatttgcccactaccaagatggcaaggtgtcgcgcttcaagccagaaaccaagtccttcctgggggaccattttgaagtctggcaagctcacgattcctgactgaagctagaagatgcagtcatggagtagcaggagagatgcaaacagaaaacgccagttagggtataaggggaaaggaagcctgcgcttgtcactatcgagactctcagaaagggcgcagctgcctctgcccttaacaaaaatggccgttctcggagggtgcctcgtcgcgccgtgaattatggcggcgcttccggtcccgctgcccccagcaaagatggcggcagtggagaaacgacggccggcagtagcaccagcggccaatttcccagacagtggccggacgtcggtgtctcaggcggtggcggcggctgagagcgaggaggatttcctacggcaggttggcgtgacggagatgctgcgcgcagccctcctgaaggtgctggagacccggcccgaagagcccatcgccttcttggcgcattacttcgagaacatgggtttgcgctcgccggccaacggaggcgccggggagcccccgggccagctcctcctgcagcaacagcgcctgggtcgcgcgctgtggcaccttcgcctggctcaccactcgcagaggacggccttcaacaacaacgtgagcgtggcctatgagtgcctgagtgcgagcggccgcaagaagaagccaggcctggatgggcgcacatacagcgagctgctgaagcgagtttgcagggacggcggggcgcctgaggaagtggtggcaccgctgctgcgcaagatccagtgccgagaccatgaggctgtgcctctgggcatcttcagaaccggcatgctgtcctgctttgtgctgctggaattcgtggcgcgcgccagcgccctcttccagctgctggaggaccctggcctagcggtggctgatcggcgtgtgggtcaggctgtgctggacacgctcgagggtgcgctgagcgcaggagacctggcaacggcgcccacgcactacctggaggccggttcccgcctgggccccgacaacctggcgcgtgccctggacagggcagcagccgggcgacgacccagtgcacccatggcccgcgaggagttcttggagaaggctgcagccctcttcattgccaaggtcaagccagtgggctaaacccacaatgcacccacaccctgtcacctgcccaggaaggcgtgaacttgggacctgcaaaccgatgtcccggcatcctgcacagggtcatcaaccaggaagggggagccacttggaccctgcaggacaagcacctgcagatcaatcccctctgtactggcctgcctaccccactcacagaaataaaatctaaaaagatccggc

SEQ ID NO: 5 - DNA, 5006 bp, Rattus norvegicus
aggtacgcgcgctgcggtatcctacccggatcgctttgcaccgggctgccggagcccgcatcctggactcagcgttcgtagagcggagcctcccactccccagcctgctatgggatggacaacgaagaagagaaccactatgtgtcacggctcagggatgtctacagcagctgtgacaccacagggaccgggtttctggatcaggaagagctgacccagctatgtactaagcttggcctggaagagcagctgccggcactccttcacattcttcttggagatggccgcttggccagggttaactttgaggaattcaaggaaggctttgtggctgtgctgtcatctgctactggcgtagaaccctctgatgaggaaggtagttcttcagagtcagctacttcctgtgctgtccctccaaagtacatgagtggctctaagtggtatggccgtcggagcctccctgagctcggtgattctgccaccacaaccaaatgtggatcagaacagcaggccaagggcagtgtgaagcctccgttgagacgttctgcatctctggaaagtgtggagagcctcaagtcagatgaagatgccgagagccctaaagaacctcagaatgaattatttgaagcacaaggccagctgcgatcctggggttgtgaggtcttcgggacaccccgaaaatcctgtagcccctccttcaacactcctgagaaccaggtccagggcatctggcatgagctgggggttggaagcagtggtcacctgaatgagcaggagctggctgtggtctgccgaagcatcgggctccacggtcttgagaaacaggaacttgaagaactgttcagcaaactggaccgggatggagatggcagagtgagtcttgcagagtttcagcttggcctgtttggtcatgagcctccttcgcttccagcgtcttccagtctgatcaagccaaatgggccttggtctcactatcagattcctgaggagagcggctgccacaccaccacgacctcgtccctcgtgtctgtgtgctcaggcctgcgcctcttctccagcgttgacgatggcagtggctttgcctttcctgagcaggtcatctccgcatgggcccaggagggcattcagaacggcagggaaatcttgcagagcctggacttcaatgtggatgagaaggtgaacctcttggagctgacctgggcccttgacaatgaactcctaacagttgacggtgtcatccagcaggcagccctagcctgctaccgccaggaactgaacttccaccaagggcaagtggagcagctggtgcaggagcgggacaaggcgaggcaggacctggagaaagctgagaagaggaacctggactttgtgagagaaatggatgactgccactcagccctggaacaactcacggaaaagaaaatcaagcacctagagcaggagtacaggggaaggctgagcctcctgcggtccgaggtggagatggagcgggagctgttctgggagcaagcccgcaggcagagagccgtgctggagcaggatgtgggccgcctccaggctgaggagaccagtctgcgtgagaaactgacactggctctcaaggaaaacagtcgattgcagaaggagatcatagaagtggtggaaaagctttcggattccgagaagctggttctccgcctgcagagtgacctccagtttgtgctgaaggacaagctggagcctcagagcatggaactcctggcccaggaagagcagttcacagccatcttgaatgattatgagcttaagtgcagggacctacaggaccgcaatgatgagttgcaagcagagctagagggcctgcgggtgcggctgcccaggagtcgacagagcccctcagggaccccaggcacccatagaaggtggacccctggacgtggcccagcagacaatctgtttgtgggtgagtctatcccagtgagtttggagacagaaataaagatgcagcagatgaaagaaaattaccaagagctcaggatgcagctggagaccaaggtaaattactatgagaaggaaattgaggtcatgaagagaaactttgagaaggataagaaggagatggagcaggcatttcagcttgaggtcagcgtgctggagggccagaaggcagatttggagaccctgtatgccaagtcacaggaggtcatcctgggcctgaaggagcagctgcaggatgcagcccgaagccctgagcctgcaccggctgggctggccccctgctgtgcacaggcgctgtgcactctggcccagcggctaggggtggagatgcacctgcggcaccaggaccagctcctgcagataaggagagaggctgaagaggagctgaaccagaagctgtcgtggctagaagctcagcatgctgcttgctgtgagagtctgtctctgcagcatcagtgtgagaaggaccagctgctgcagacacatctgcagcgagtaaaggacctggctgctcagcttgacctggagaaggggtggcgggaggagagggagcaggaggtcctggcacactgtcggaggcagcagctgaagctgcaggctgtgatgagtgaagagcaggcgcggatctgcaggtcgttcaccctggagaaggagaagctagaacagacctacagggagcaagtggagggacttgtccaggaggcagatgtgctgcgtgctctcctgaagaatggaaccactgtggtgagtgaccagcaggaaaggatacccggctccatgtacccgggtccagacagcaggcagcagccaccaacctggcaaactgtcagccccgatgggaggacaggagcaccagcagaatggcctggcccagggagagctgatggcagagatcttcctggccagctctgtagtttagatgccgtaccaagcccaacccctaccctcctgtccagaaggtcctcagagagcctggatgtcagagacaaccatcaaggtccactcagtgctgaggagggagctgtcccaaaggagccagaaccttctgccaggaccctaacaggccaggaccagaagctgcccttgcctattcagccacagatgttggagccatggttgggtccagctgctgtggacaggaagccagactctgttagggttcagggacaggcttcagaagggcctactggggatgataaaggtgtccaagagactccactccagctgaggggagaaactgcaaggatgagaccctcattgccctactctgagctcccaaacccacaagaggccaaagtgatgtcagtgatgtcagagagtgagatgaatgatgtgaagaccaaacttctgcagctggaagacgttgtccgggctcttgaaaaagcagattccagagagagttacagggctgagttacagaggctttctgaagaaaactcagtgttgaaaagtgacctggggaagattcaactggaactgggaacttcagagagcagaaatgaagtgcagaggcaggaaattgaggtcttaaagagagacaaggagcaagcctgctttgacctggaagagctcagcacacagactcagaaatacaaggatgaaatgtcacagctcaactgcaggatccttcagctggagggagattcttctggtctccatacccagaaggaagaaaatcacgctgctatccaggtgttaatgaagaagctggaggaggcagaatgccgggagaagcagcagggtgatcaaatcaaacacctgaaaattgaacttgaacgtgtgaatgaggaatgccaacgcttaagactgtcacaggcagagctgacaggaagcctcgaagaaagtcaaggccagctgcacagtgtccagctgcggctggaggcagcacaatcccagcatgaccggattgtccagggcctgcaggaacagatgagtcagttggttcctggagctcgtgtagctgagctgcagcacctgctcagcctcagggaagaggaggctgagcgactaaatgcacagcaggaagaatacaagcagcagctgaaggccagggaggaccaggtggaggaggccgaggctcggttacacaatgtggagtggctgctacaggagaaggtggaggagcttcgaaaacagtttgagaagaataccagatctgatctgctgctcaaagaactctatgtggaaaatgcccacctcatgaaagctgttcagctcacagaagagaagcagcgaggagccgagaagaaaaactgcgtcttagaagaaaaagttcgagctctcaacaaactgatcagtaagatggcaccagcatcactttctgtgtagggactgctgttcttggagttcatgtgaaggaatgtctttttaaataatgctactctgatgccataattttctatggctcactagctacactccctaggcatgaggactgattcctgttcatctgacaaacattcaatgaatgtctactaggtgccaggttctggggaacaggcagggtacagtcactggtggccaagagtcttgggtaacatcctgggttgcaatctccaagttgatcttactggaaaagactcagagcctcagcaagaacaatggagagagagtggctcagtacttgaatgtggggctttgggccagaaacgcagggtcgggatttaggtgtcttaccacttcactgccctgtgtgggctgtcatcagagctctccgtgtgggtcagtacacccccaccccacccccagaaagagggccttcccctgcagtcctctgctgctcctgagggtgggagcaagatggcatccgaggtggtgggctcagggatagcagtgttcgttgcgagtgcaatctcactacaaaatatgttgtcacactggaaattgtacatttttttttaaaagaaaaggtatagttttataactgagatgtacctaaattatgaccatttggtttacgagtattctactgtgatatgaaaagttcctgtattttcaataacttttctattaaaattgttttaactctt

SEQ ID NO: 6 - DNA, 545 bp, Rattus sp.
ttggccctcgaggcctagaattctgcacgagggcttagagtgcatgggctatagcctgaaatgatctcaaactggaaaaggatttttgttgttgttgttgttgttgctattgcctgtctcccccaacaactgttttcaaatggacggggtcttatttacaggagggctccctactaagttcactgtgggtgcccacgtgcgctctgtggtgtaaagcatccgcttactggctacacagactgcctgcagcagtcatgccatcataacgcctgcctgcctgcctcagatccccagttaacaccccaattagagaccatcaggggcggtgaaaagtgcttattgagcattctgtccctaaagtttaacttataaaaaacaaactgtgagcggctgtttgaatctttgtttttgaaatcagctaagaactgacaaactctgtcatcgctgagccaccgagatcgttatcagtgacaaccttgctcttgtgatctaaagacatttgtgatcttcacccggacattcacttaaaaaaacaattgcgggcg

SEQ ID NO: 7 - DNA, 1258 bp, Rattus norvegicus
atgagggcttcctctagagaaaacgtggcagggtcgtccataggattgacctcctggggcccgaccacccctggggtgacgccagcaggcagcacgactgattctgaggagaatgaaaggattctgtctccagatatactcaccactaaccagatcattgacctgctgggagaagaggacgtggaaaaaattcaagaaaaaatgggaacgtttctgaattttaaaaatcttcagactagtctacgagacgccattctattagactactatgtggctgggttctgctgggccaagggaatgaacttttctattgaccagtattcaaaatttatgactttgctggacatgttacttcaaaatcttcaaacgctgcatatgtccttagaggacagcatcaagtggctcggggaagtgatggcagaaattggaccaaaccattcgcagaagaataagaattttcatatcttcaatgtcaaggaagccaatgccatcatcgattatttaaaaatcagcttattccagcactacaggctctacgagtttctgttctactcggccagggaagaaattgtcatcgggacggagcaaaccatagaggccgtcaagcctgcagaccaccctttcccagcccctctggaagaaggtatctcactgagcatgtattcagccttcatagagccgttgccaacgctggacactgaacagaaggggctggatcaagagcaagggcctcaggaatcgctgctggagagtgaaatgggagaagaggatcccttaggtggcttcaccattgatgacgtaaaattagccctggcacgagtgacggatgaggtcttgataagcattcagaacgagataaacgaaaaactgcaagtacaggaagagtcctttaacgcgcgaatagagaaactgaagaaagcctgagaatgcagtacaagttaatcttccgagaaaccaacaaaaccggccagaataataatgtctctatagcactatgtcacctccatgaagccctggaaggaaaacccacttcccgttatcctgagtgattgacaaggcgtcgtcccgcccctcccattccacacccacctctgagtttacagggccaaaagcaagaagagcattgctttgtatgattttggaagttctatacgaaagtactattgttttgactaatgaatactattcctctttctcggaaatgaaactaaccttttgaaataacaataaaaaaacgtagtaaagtaatagtaccttt

SEQ ID NO: 8 - DNA, 3019 bp, Rattus norvegicus
gagacaatcgcgccgagcgggcgccgcagccggagcgggagcccaacccggagctgcacagagcggtgcagagctggggctgagcggggccggccgagcagggccgggcggcagcgatggaccgctagggcccggccgagccccgcgatgccgccgccaagtggccccggagtcctcgcgcggctgctgccgctgctggggctgctgctcggcggcgcctcccgggctcccggcaagtctccgccggagccccccagccctcaggagatcctgatcaaagtacaggtatatgtgagcggggagctggtgcccctggcccgggcctcagtagacgtgtttgggaaccgcactctactggctgctggtaccacggactcagagggcgtggccacactacccctcagttaccgcctgggtacctgggtgttggtcactgctgcccgtccgggtttcctcaccaactcagtgccgtggcgtgttgacaagctgccgttgtatgcatccgtcagcctctacctgctcccagagcggccagccaccctcatcctctatgaggaccttgtgcacatccttctaggctctccaggtgcccgctcccagccctgggtgcagttccagcgtagagctgcccgcctgcccgtcagctccacctacaaccagctttgggcttcgctcacccctgccagcacccagcaggaaatgcgggctttccctgccttcctggggactgaggcctctagctcaggcaatggctcctggctggagctgatacccctgactgctgtgagcgtacacctgctaacgggcaatgggacagaggtgcctctctcaggccccattcacctgtccctgccagtaccctcagagcctcgtgcccttgctgtgggcaccagcattccagcctggaggtttgaccccaagagtgggctgtgggtgcgaaatggcactggtgtgattcggaaggaaggccgacagctctactggacttttgtctccccgcagctagggtactgggtagctgccatggcctcccctacatctgggctggttaccatcacctcaggcattcaggacatcggcacgtaccacacaatcttcctgttgaccatcctggcagccctagccctgctggttctcattctgctgtgtcttctgatctactactgccgacgacgctgcttgaaaccaagacaacagcaccgcaagctgcagctctccggcccctcggacaacaaacgagatcaggctacgtcgatgtctcaactccatcttatctgcgggggacccctggagcctacgtcgtcgggagaccccgaggcccctcccccaggctccctacactcggccttctccagctcccgagacttagcatcctcccgggatgacttcttccgtgccaaaccgcgatccgccagtcgaccagccgccgagcctcctggtgcccgcagcgttgaaggtgccgggcttaagggcgcccgctccgtcgagggtcccggcgtgctggagcccagcctggaagaataccggcggggtccagcgggagccgctgccttcctgcatgaggccccctcgccgccgccgtccttcgaccattatctgggccacaagggggcggccgagagcaagacccccgactttctgctgtcacagtcggtggaccagttggcgcggccgccgtccctcagccagcccggccagctcattttctgcggttccatcgaccacctcaaggacaacgtgtaccgcaatgtcatgcccaccctggtcatccccgcacactatgtgcgcctgggcggagaggcgggcgccgcgggagtgggcgacgaggccaccccgccggagggctcggccgccggccccgcgcgccccttccctcaacctgatccccagcgcccgctgatacagggccacgcaggtgcgggaggcgacggcggtggcggcgagggctggggcggcgggcgctccgcaccggtcagcggttcggtcaccattccggtgctgttcaacgagtccaccatggcgcagctcaacggcgagctgcaggctctgaccgagaagaagctgctggaactgggcgtgaagcctcacccgcgcgcctggttcgtgtccctggacggacgttccaactcgcaagtgcgccactcctacatcgacctgcaggcgggcggcggcggtcgcagcacagatgccagcctggactccccgcgcctggcgctcagcgaagacacagagcccagcagcagtgagagccgcacaggcctctgctcgccagaggacaactcgctaacgcccctgctggacgaggtggtggcacccgaaggccgggcagccacagtacccaggggtcggggccgcagccgtggggacagttcccgcagtagcgccagtgagctgcggcgcgactcgctcaccagccccgaggacgagctaggggcggaggtgggtgatgaggctggcgacaaaaagagcccgtggcagcggcgggaggagcggccgctaatggtgttcaatgtcaagtagtgcggcccgggggtcgcccaccgcccctgccgccagtgcagattcccagggtgcgcgccccgggatgctgcagcagaggggcatacgctcttagcccggagaagagctcatgggtcagctgagctcatctgtggtttgtgctgggggagtgtcttgggaagggaccctggattggcctctaagcagagggaggtgggagggacccggtcccctaagggaggggctggggaggagggcaggggtggggagcagtgcctgtataaacgtggctgtacatagaaagatctattgtgggagcaggaggggcaagccagcctcagctgctagggtgagggctgggaagggagggacaatctctgtggacccttgggaggggaggggtgctgcttggatgtcacagggctggtggctggggcggtcctggggactataggataatgtgatgacactctggacaacaacaacaaaaaaaaatacaaaactggtgaggttaaagctc

SEQ ID NO: 9 - DNA, 933 bp, Rattus norvegicus
acagcactggcaaaaccttgtctactcacagagccctggggcttctcagaggaaatctgtgaggtggagtgaaaagccagcgactctgcattatgcctgcataccaagaagacagagaggaagacacatgtactattggggagggagaaaagaacattgctactcaacgaatcaagggatttggtggggagaacccagtccttcctgaaagccctcccgcatccatgtcttcagtgatgaagaacaacccgttgtatggtgacgtgagtttggaagaagccatggaagaacggaaaaagaatccttcatggaccattgaggaatacaacaagcactctgtgcattccaatctctctggacatctgaaggaaaaccccaatgacctgcgcttttggttgggggacatgtatactccaggctttgacactttgctgaagaagaaaaagaaacgcaacaagcgatcaaaattatgtcacatgggtctgattttactcgtcgtgtcctccatcctggttaccgtagtgactctcagcgctttattcgcttaaggaaattgcaagcccacctttgaagctctaaaacgccttctaaatatttgcaggaaaatctgtgcgtcgtgaacatctgccttgttaactatgtgactaaatgccactgccctttagacgtgagcgttgcctgtattatgttggcctctctgctttgttctgtgaaacctgtgtgttattccagtctgggtctgtcctttgtgaagcaggtatttcagaggctcacagtagaaaaagttactccacacagtcacaaccaagcttagataacagactgcatcaagtgctggcagctgcttgtcttgtgcttacctggttttcacatcagtcctccttctcctcctctttctcctcctcctcctcttcttcctcctcctcctcctcc

SEQ ID NO: 10 - DNA, 2302 bp, Rattus norvegicus
ggttccctctgtgtgcgctggatgcaggggatagatcccttttggctcagttgaccgggttctagcgtccctgcagcccggtgccttgggctccagggtgctaggcgtccccaccgcccctcctctcctggagggcggcagggggcggtgcctggcagtgagctaggttcattggcgagggagcagtcgccgggcagcggggcgcgggacacacggggccgcggaggcgccctccatcgccatggacctgatatcatcgctgagcctgggcgagctagcgctcagcttctcgcgggtgccgctcttccctgtcttcgacctcagctacttcatcgtctccatcatctacctgaagtatgagccagggtctgtggagctgtcccggcgtcacccggtggcatcctggctgtgcgccatgctgcactgttttgggagttacatcctggccgacctgcttcttggggagcccatcattgactacttcagcaacagctccagtatcctgctggcctccggagtgtggtacttgattttcttctgtcccttggacctcttctacaagtgcgtctgctttctgcctgtaaaactcatctttgtggctatgaaggaggtggtgagggtccgaaagattgctgtgggtatccatcacgcacaccaccactaccaccatgggtggttcatcatgatcgccaccggatgggtcaaaggctctggcgttgcccttctgtccaacttggaacagctgcttcgaggcgtctggaagccagagaccaatgagatcctacacatgtccttccccactaaggccagtctgtatggagccatcctgttcaccttgcaacagacccgctggctcccagtgtccaaggccagcctcatcttcgtcttcaccatgtttatggtgtcgtgtaaggtgttcctgacagccactcactcccatagttctccatttgatgtcctggagggttacatctgcccagtgctgtttggggcaacttggggaggcgaccatcaccatgacaaccatggagcaccacatggcatggggctaggcacccagcactctggcttgcctgctaaggccaaggaagagctgagcgaaggcttcaggaagaagaagaccaagaaagcggattagggttggccccagggatcgatccactgggtgcttgggagaaggatatgaactcagagaaactcagaacttagggcattcagggctcggaaacccccctccccccaccagcctcaacttccccatctacaatttggagccatcagcaaccccagggctgatgtggggttaaattaatgtggaggagtttggtaggagtggaaaaccctgccttctgccagactcctcatccggtcacaggctttgggggatacacagtccaggcctaaaggctggtaggttcttggttttcattctgtgttctaattatatcttgaaaccaaaacaatttaaagtcacgtggagactcaattatccaagttcctcctgggagccaggagctggggtcctgcgtcctttcccaccctccctagaattttatgactcttgctgggcattcaggcaaatggcctcccttctctgagccttctgtttccattctgtagcaccaagcccccaggtcctcctgtccgcaggaggtctttacaatgtcaaacagtttaacagtgctaataaggagactggagctggcatgaagggcagtggcagactgctttgtatgtcaggccatttgtgttggaacccaggtcacctcactagatactcagctgagtagacacatctagggctgtctgttaacgggtgttgggagtggggtgtgttgttgagttcccagctgcccacaggcaaaagaagtgtgtgtgccgggcagagggtccaagagccaatagccttgagtgctcacaattgggccctgggtgtatcaggtgtccctcgtgtctgatcagcatagttcactcagtcattcaacaaacacacagatccacacatgctccctgtacagtgttgtgcggcttggaggaggagcgagccttgtacagggcaagggccatggtcaccaaagtctccaggacctctgagagaggtgagcatggggtactgctggtctcaggcttctgtgaggctcagatctagttggagtacccagtgttgcccatttgttgagaaaacgctcctcacaacttttcctggaggtaggtcctgaggctggaagctcctgggtttcatccccttggtgtcaatcaaagctctgtgtgagtgatcagccaataaatctggtgcgagtga

SEQ ID NO: 11 - DNA, 1680 bp, Rattus norvegicus
aggctgcagaagcaaatgctccggaccaatcatggggcacggactcagcagtatctcagcctcggagctggacaagttcatagaggtttacctccttccaaacaccagctttggtgctgacgtcaaattagcgatcaatgtcgtgtgtgatttcctgaaggagagatgcttccgaggtgctgcccacccagtgagggtctccaaggtggtgaagggtggctcctcaggcaaaggcaccacactcaagggcaagtcagacgctgacctggtggtgttccttaacaatctcaccagctttgaggatcagttaaacagacggggagagttcatcaaggaaattaagaaacagctgtatgaggttcaacgtgaaagacattttggagtgaagtttgaggtccagagttcatggtggcccaacccccgggctctgagcttcaagctgagtgcaccacacctccaacaggaggtggagtttgatgtgcttccagcctatgatgtcctaggtcatgtaagcatctacagcatgcctgacccccaaatctatgccagtctcatcaggaagtgcatgtacctggggaaggagggcgagttctctacctgcttcacggagctccagaggaacttcctgaagcggcgcccaaccaagctgaagagtctcatccgcctggtcaagtactggtaccatctgtgtaaggagaagctggggaagccgctgcccccacagtacgccctggagctgctcacggtctatgcctgggagcgtggaaatggatttgtcgattttgagacagcccagggcttccgggcagtcttggaactgatcataaagtaccaggagcttagaatctactggacaacctattataactttcagcaccaagaggtctccaactacctgcacacacagctcacaagaatcaggcctgtgatcctggacccggctgacccaacaggaaacattgctggttcgaacccagagggctggaggcgactagcaggagaggctgctgcctggctgcggtacccatgctttaagtacaaggacggttccccagtgtgtccctgggatgtgccgatggaggttgacgtgccgtaccaggaggatcacttttttcgtaatttttgtctattttttttgtttttgttccttttcatattttggagggtttcttgtgtatagtgcaggctctcgtgtatatcgtcatcctcctgcctcggtgctggcatgactgcagagtccgcctgatgtgccctggattccctccatcctcaagtggacaagactgtgcatctgtcgtcctgtgagcccagcaggacctgcccaggaggctccagagtcagtcatggctttctgtgctgcaggcccttgacccagagagggaaggaaggttcccaagaccccagtgagggagggtccaacctgtgatcagactctggtcttctgacccctgccttcctactcctgcatcctgtcccatcacagacagccctcctcacagcctgcttcatctgccttgtcctccaacagtgctctcttgggagacaagagattcagaaggggaggcaggaactcgagcttgacttccacctgtccacctgttgggagttctgtccaatgtgtgaccaacgacaataaaccatagcaagcaaaaaaaaaaaaaaaaaaaaaaaaaaaaaaaa

SEQ ID NO: 12 - DNA, 3628 bp, Rattus norvegicus
tggggatgctagtgcctactgcgctggctgctcggctgctgagcctgttccagcaacagctgggttccctctggagtggcctggccatgctgttctgctggctgagaatagcattggggtggccagatcctggaaagggacagccacgggtccggggtgagcccaaggagactcaggagacccacgaagatccaggcagtgctcagcccacaacccccgttagtgtcaactaccacttcacgcgtcagtgcaactacaaatgtggcttctgcttccacacggccaagacatccttcgtgctgcccctggaggaggccaagcgaggactgcttctgctcaaacaggctggtatggagaagatcaacttttcaggaggagaacccttcctacaggacaggggtgaatacttgggcaagctcgtgagattctgcaaggaggagctagccctgccctctgtgagcatagtgagcaatggcagccttatccgagagagatggttcaaggactatggagactatttggacattcttgctatctcctgtgacagctttgatgagcaggttaatgttctcattggtcgtggccaagggaaaaagaaccatgtggaaaaccttcaaaagctgcggaagtggtgcagggattacaaggtggctttcaagatcaattccgtcattaatcgcttcaacgtggacgaagatatgaatgagcacatcaaggcgctgagccctgtccgctggaaggttttccaatgcctcctgattgagggtgagaactcaggagaagatgccctgagggaagcagaaagatttcttataagcaatgaagaatttgaagcattcttacagcgtcacaaagatgtgtcctgcttggtgcctgaatctaaccagaagatgaaagactcctaccttatcctggatgaatatatgcgctttttgaactgtaccggtggccggaaggacccttccaggtccatcctggatgttggcgtggaggaagcgatcaaattcagtgggtttgatgagaagatgtttctgaagcgcggggggaagtatgtatggagtaaggctgacctgaagctagactggtgaggtgagattggacggagactctaaccagctacgaggagtccacgagcagccgctcgccaccacgcacatctggctgtctacggactgttgctactgagaatgcactctatgttctttctttcagtttgactgaactaagaaaggagtaaaaccattctgtgaacccttatacagccgataagggtcctccacaggggaaggagttcagttcacagaggaacaaggtccagcgatctcaaagaactcacattttatttgtattcgaatttttgtatggatcgttactgtgaatcccatgtttccttttccctttctgttcccccatgagaaactgggttattttctgctaactatgccagttttgtcaacaggaaggatcacaaaacacaccctgcgattactgctgactgttctaatgcttttctgctttggaatctcggactttttgctcagaaatatagaatcaagaagtgtgttatagagccgtacgtgagaaccttactcttttgcaaggagcatatggaaggaggtacttctgaggacggtgtgttcatggcctcagcctctgctgctgctgctgcttcaaggatggctgttctatatcgtaccaattccttcaaggttctggacaaagccaaggtacactttacctttcatggcatggccgtgaagatggcactgacagccgcaaccaccccagcactgaggctgcctgagtctgtcaggctaaaaggttgtcccacttttctgaaaagctacaagggctcttctgcacatccagcagaatcatgacaggatgggcatggaggactcttcaggagtatgaaatgtggctgctgctccttaccatatttctcatgaaactctgaaataagacctagtagtctaggtcaccatgagaaaataatttcttctctacatcaaataagcaaaagagagaacatgaactagcccaaatcttcttcctgtggaagcattgcatgagtttattcaacctagacagcctcactgcttggaaaataattccctggaaattaaataaataccatggaaaagaaattaaactcctctgcaatcccactgtggccactagagggcaccaggaagtcagacttcattgtctcagagacttttcttcagtctttttttagaatttggtgaattttaattctgttccttatctgttttgtctttgttttttgttttggtgcctattactaaaattaatactaaacattttccaaggaactggaattttggccatttacaaaaggtcttcattctttagtgaggtcactataattcaactaagacatactctgtctcattaaaaataggtcaggttttcctgtgttttctgtcttgtttggttgtgtgtgtgtgtttacctatttttgcttgaatatagatataactatgtatcccaggctagcctggaactcatgatacctctgccttaacctccccctcttaagtgcccaggaattacccaactcctgtacagagatttttaagagaagtttgagtcacattcccccctgcagattgtgatttttttttttaagttaagtcctgtgtacatagttcacatgggaaaccatgacatcccagaggacatgggcagacacccacttggtcacacgacagagggaagctgcatgtttctttccctaattaaagtagagcatgaagaaagtatgattggaaagtgagccatcatttacatgtacccccaaccccttgtgcatgcactggcggtgggagatctctgtgcttaaaatattgaaaagtcccctttccttaaagggaaaaattacgatattttactatcgtagtacaaatcaaacttggattagatcatacattggtttgctttttcagtgttaaatktgggtgttcattttgtaaagcatacgctccctgctggggaaacaaggagtgatctagttgacatttgttaagtgtgaccagcagataccttgtgtcccatgattgatttatgcagagcagtaatactgggaagggggatttacttaagaacatcgacgggtaggacatccaacactgatttcaaagttttggcttctttattataatgtgttcggcggtaaaaaaaagtcatgggttgttttctttgaagctgaaagtctcatggtgttatttacctaaattatctgaaaaatttaaaaggggcccaggtttcattctgcgttggagtttgggggtctaattgttgcataaataaacaggcaaccaatcatcagaggttgactcctcccatcaataattcagctcgtgttttacccttttgatggactgatgttgtttcataagctattagtagtccaagaacaatcatgcaggcctcatgtgaatgtgttcagatgaggtttgatgttcgggatacatgtgcatgtatctccctgtagaatgacactgtattttgaggcattgctctagtgtatggaaacgtgatctgtgtattaaaatgttttatgcatgacctcaaaaaaaaaaaaaaaaaaaaaaaaaaaaaaaga

SEQ ID NO: 13 - DNA, 1098 bp, Rattus norvegicus
atgggcactccagagaagaaatgcttccttgagactgtgtttttctacctcctggatacgaggagcctagaggacctgcagctcctgctgactgaagaggaatcctggagacattttgtggcaggagttggtttgtccagggaagaggaagatgccttgcgagaagctcttgctgagatctttgaagattctgatggagaagaagaagacaaactccaaagtgaccctcaggacaagaaagagaggaaagaggaagatgccttgagggaggctcttggtgggaatacagctgactctgatacagaagatgaagaccaactccaaaatgacaaggaaaggttcttggaggcttatcctcaggtgaggctggagctcgagaagcacatcaagaagctccacgccctggcagacaaggttgacaaggtgcacagggactgcaccgtctcacaggtggtggccagctccagcagtgctgtgtctggagtcctcacaatccttggcctggctctggcacctgtgacagcaggagtcagtctggcactgtcagccactggcatggggctgggagcagcagcggctgtgactagtgtttccacaagcattgtggagaaggtaagcctggcgtctgctgaagctgaagccagcaagctggtgccaaccaaagacacgatgaacaggatgaaagaagttttggagcagagtggccccagacttgcttctttatctacaaattccatccagaacatacaagccatccagaagaacatgaatgccattcagctggccaaagccaaccctcacctagcaactaatgccaagcgtctcatgaccacagggaagacatcaacccaaaccactggacaggtgcagaaagcctttggaggtacagctctggcaatgaccaaagaagcccggatcataggtgcagccactgtaggtcttttcctcctgatggatgtggtcaggcttgtggaagactcaaagcatttgcatgagggagctaagtcagtgtcagctgcagagctgcgacggcaggctcgggacctggagcagaagctgcaggagctcaaccaggttcacgacagcctgactcagtga

SEQ ID NO: 14 - DNA, 1928 bp, Rattus norvegicus
aggagcaagtctggaggactgtctgcctttgttgttcttccctctgtaatccactggctatagcttagaaacagacccatcattaagagactacatggactgtggagtcaatgggagaaagtgaaatactggagtcaaggaagctaacagcaatagaagcttgaagtttcccatcatcacattgtatggattggaattgaggaagaagacagagagctgcctactaaccagggtcttagctttcacctctgagaatgctgttctccgatgacttcagtacttgggagcagagatttcaagaactgatccgggaggagaagcccggggccaagtggaccctgcacttggataagaacattgtaccagatggtgcagctctgggatggaggcagcagcagcagacagtagttggcaggttccagtgttccagatgttacagaagttggacctctgctcaagtgaagatcgtatgccacatgttccaggatgctgggaaaacacagggccgggtactcatgaggatctttggtcagaggtgccagaagtgttttgggtctcaatttgagagccctgagttctccacagagagcattgaaaggatgctgaataacctggttaattatattctgcaaagatactacggacataggaagatagcatcaatcttgaatgcatctttggatgaaaaggtgcttttggatgggccccatgacacattaaattgcgaggcatgcagtctgcacccccatggaaggtgtgccgttgcacagagagtgaggccacccagatccccatctccatctccatcgccatcaccaaagagccacagttcctctccatcaaggagccaccctcctttacttcagactgagattacaggttttaggaacaggccttttcaggaagtcagagagcccggtgaagcagcatcaccactattagtgattttgtctgtcgctgcgcttgcccttattggtttcttcattagataataggtttatcatcttgttttaatcccgtggtgatcaataaaatggatgtcatttcaacgtgacagtgaattcatgtgggatcgtatagagaataaacctgctttcctgggcatgctctgaaaatggcctaaaacatggcccacacataatggatccaataacaaatctgtgttgaattgctgaactgtctgtactagctttcacacttactgttctcaagcagtgttgattaaatcttgatgataactaacagtcatgagtgttatgaagtacagagtggtggcaggagtaagggggcacctagtaggcccctgccaaggagtcccccttagaaatcatgccatgtgacagatctggtgtaagggagttttcttgagggggaaatggggagcagggacctggagaagaggggagcagagctatgaggacagagaaggggggacagggaaagacagagagagagagagagagagagagagagagagagagagagagagagagagagagagagaggcaagccaggaacacatttggaaaaaagaaccaagggttctaggagccctagaaaagtccctgggattctggaaaaagagggcttgactttccagcctgccatctttattgtctctgacaaggagaggatacctgaaggtggtttaggactacagagaagttagccatgaaggaaagaggagaaggggggggggagaaggagaagggggcaggaagtgagcctgaggcacctggctcccacccagtgggcagcaggtaatgatgtaagcctaggtccaggggagaagcttagtgtaatcttctgttaaccaatgaagagtattatcataaatttactgtggcagactgggtaatgcttcccccaccaaaaatgcccttccccatatcctctagaatttgttaataaactgcatttgttggaag

SEQ ID NO: 15 - DNA, 5632 bp, Rattus norvegicus
atgaacaagatcctgtgtacagcaaggtttgtgtgggaacaaacacaggactgtcaagaagcacgcatgttgactgtgtttaatttagtgctgaagaaacggccatgcctgcggcccccgtggctgctgccaccactgctgctcctgcagctgcttgctgctgccccagccaaggccgaagcaaaggaagagtcggaggaatcagatgaggaccaacccacactgccttacaactttgtagtaagctgcgactgttgtgtctatgtggccgtggggattgaaccttggacccagggcaagctagtgtcctgtgcacttacagccatgggtcagtcttcttctaaacctgatgcaaaggcccataatatggcctccagctttaatgagttcttcaagagtttcaaaatggaaagtaaaatcctttctgaggagaccatcaattcaattcagtcgtgtgtgcaagaaggagacatacagaagggaatttctataatcaatgctgccttggcagacattgagaaggcccccctgaacatcgcagtgacaggggagacgggggcaggaaagtccactttcattaatgccctgaggggagtagggcatgaagagagtgagtcagctaagattggagcagtggagacaaccatggataagttccctaagttccctaacgtgaccatctgggacctccctggggtcgggacatgtaacttcaaaccagaagaatatctgaagaagctgcggttccaggagtatgacttcttccttatcatctcagctactcgctttagagagaatgatgcccagctggccaaagcaatcaaaaaaatgaaaaagaacttctattttgttcgaacaaaaattgacagtgatttgtggaatcagaagaagtgtaaacccaagtcctacaataaggaaaaaatcctggaggaaattcgcaaagactgtgtggagaagctgcagaacgctcgggtggcctctgctcgcgtcttcttagtctccagcgttgaggtagcacagtttgactttcctgagctggagtccacccttttggaagagctgccagcgcacaagcgtcatgtcttcatgcagtgcctccctagcattaccgagagggctattgaccgcaggagagatgccctgagacagaagatctggttggaggctctgaagtatggcgcgtcggccaccatccccatgatgtgtttcttcaatgatgacatcgaggagcttgagaagatcctgacccactacaggggtagctttgggctggatgacgagtcgctgaaaaacatggccagtgagtggtccatgtctgtggaggagctgaagtccttcattaactcaccccatttgctgtcatgtgagatgaatgaatctgtgtcagacaagatggtgaaaccctaccgggcagagctctaccgggtcactatcccccagcatagagctgccatccaggataggacctggacaggagtgcagagagtcacctttgtcccaggacagcaggagaccaaggaggcaattccctcagagccacagaaagtctccatgtcacagggagacaactggggtgtttttacccctttcataaacatggcgaaacctctcaagccgccattgtttaaatccatcactgctggtgagtcatcctatagcagccagaactcttcttctccagaagtcattgagaaggtcggtaaggctgtggcagagggggatttacagaaagtgatatacacagtcaaagaggaaatgcagagtaagtctagatacacggtaaaaatcgccgtgactggggactctggcaatggcatgtcatctttcgtcaacgcccttaggctcattggacatgaggaggaggattcagctcccactggggtggtgaggaccacccagaaaccagcctgttactcctctttccactttccctatgtggagctgtgggacctgcctggcaccggggtcacagcccagagcatggagagctacctggatgagatgcagttcagcgcatatgaccttatcatcatcattgcttctgagcagttcagctcgaatcatgtgaagctggccgaagccatgcagaggatgagaaagaggttctatgtcgtctggaccaagctggacagggacatcagcacaagtaccttccctgaaccccagctcctgcagagtatccaaaagaatatcagggagaatctccagaaggctcaggtgagggacccccccatatttctggtctcctgctttagtccatcttttcacgacttcctagaccttagagagacactgcgaaaagacatccacaacatcaggtacagagatcccttagagaccctttctcaagtctgcgacaagtgcatcaacaataaggcgctctctctgaaggaggacctgatgttcacgaaacacctggaggcagctgtcagccccccgtatgatattgctgacctggagaggagtctggacacctaccagaagctctttggtgtggataatgagtcacttaggagggtagctcagagtacagggagaccagagatgagcaccagggccttgcagttccaggacttgatcaagatggacaggagactgaggttgatgatgtgttttgtcgtgaacatactcctcagggttcttggaagtccatggtggttcggcttgtgggatgtcgttacccgatacttcagacaccagagacagaagcgcatcattgaaatagttgctaagaacaccaagacctccttgaggagagctctagaggactatacacttcctcctgaaatcctttgtgaaggctgcctggaagacagcagttttctcccgttcacctttggaacacagagccatgcctacctggatgctgccatgcttctggccttgcctgaacctctgaacctgggccaagaggtgggtggcttccggcaatttcgtgctgatagcagcaggggattagaaaaagaaacccagttacttgggctctttctctcactgcctgatagaattgggttaactctttgtgagccagtgagaaaggagaaaaaaggaagcacactgggctcctgggaggaagccactgacaataagccttcatcagtgctggcccccgacttggggaggttctggaccccagccggcaagacggtgaagtctacatccagggttgctccattgctcaccagcatggaagaagcagtcgggttgcccgaggataaacagtttgcatgcttatccgacgctgtattcatttccaaagacaacagtattttatctgtagaagtcatcaagagtattcaggctgctgtggcgggagggaacggggtggaagtggtctctatagttaaagagattgtgcagaaagtatccagaaccacaatgaaaatcgctgtgactggggactctggcaatggcatgtcatctttcgtcaacgcccttaggctcattggacatgaggaggaggattcagctcccactggggtggtgaggaccacccagaaaccagcctgttactcctcttcccactttccctatgtggagctgtgggacctgcctggcatagggaccacagcccagagcatggagagctacctggatgagatgcagttcagcgcatatgaccttatcatcatcattgcttctgagcagttcagctcgaatcatgtgaagctggccgaagccatgcagaggatgagaaagaagttctatgtcgtctggaccaagctggacagggacatcagcacaagtaccttccctgaaccccagctcctgcagagtatccaaaagaatattagggagaatctccagaaggggaaagtgaaggagccccccatattcctggtatctatcatgaagcctttattacatgacttcgaaaggcttagggagaccctacggaaagacctctctgacatcaagtaccatggtctcttagaaaccctttaccaaatttgtgagaatactattaatgagagagtagagtccattaaaaagatcatagatgaaaataacctacaaagagagtttggaatcttgactccagacaacctgacagagactcggaaagtcttccaagaaatctttggtgtggatgaccaatctctcagccaggtgtctcggagtatggaaaagccagatacacattacaaggctagcatagagtcccaggagatacaggggtaccaacaagatggctggccattggtttggttgcatcgccctgtaatccagtttttctctacaggccttgaccgcgtgccatgctgtttttactctcctcaccatagatacacgcaacagaaaggtgtacttgatgaaactgctggcaaaaccaagaattttctgtggaaaatcctgaaagactccatttctcatcttcaaaagacctagacaactgaggtcttctccccagccctgcacctcacatgtacccccagttgagctaatccctgccccaggggttccaagttccttgaggttaataggctgactctcctaatggtgcctcatgaggcactataatggactttggtctgtaccatttgtttggttttactaattgaactatcaggatcccactgtaagagaaatttcccttttctaattgctcagaatttcacatttgacacaagctcaccttagcgcctatgattaatatagttcctcttagaggcctcttggtttgctcatatctctttcctacttttcttccctgactctggagatggagtatcccttaaggaatggggaagaccatggccagcagaggcctgttctctgaccccctcctctgatctgtaaagaggaaaagggagttaactctggtcctactggtaatgggatttgcacagtgctgaacagaagggttcttggtgccctacactaccctcgatgatgtcagtggtaccatgactagtcatggcccctcacactcttgtgctcccagttttatattggacctcaacttcttcagctgcccctaactctgcaggccgcacccccatccacatacacacaggcccttctcccgtttcgttctgtttgtgtggctttgtgtttccttaagacaggtgccactccgggttctgtcatgttcctttctctcagcccggctttcagtagacatcagtactgtcagcccttctcagtctcagacctttctctgtctttccctttcatctctttgacagatcactaaatgctttcccactgccaactgttaccagcctcaccccggagtgtttagtttgggctcatagtttgccaggacagcccatcacagctgaaagtcatcacaataggggcacggagcagctggtcccatgtgtcctcggtcaggaagcagagagagatgaataggtctctttagtacaggtttgccttttcattcagcacaagaccccagcctatagaacgataccatccacgttcaaagtgggtctcccaactttcaggtgtagtttcacacatgtgtagggaactgtggtttcagtttccaagtgtagaaactccctctagacatgcccaaaggtccctttccatagtgattctagatcctgccaaattgacaaccaatattaaccacacaggcactgcgggtatacaggggttttattggttaatagtgggaaga

SEQ ID NO: 16 - DNA, 520 bp, Rattus norvegicus
ttttttttttttttttttcaacttccacaaaatcttctgtttatttctttcagaaacatatccaaactttaacacaacccattaagactacttgatcgggtttgcagtccccttacgccacctctgcttttggtggtgatgttggttttctcacgtgcctgttaccaacaacgcaaggcttctgatgacaacgtttaccttcaactttgctcttgattaaattaagggtgcataaagagttcaggaatgtttgcaggataatacaaattgggagactctctaggtataatggttaatgatttgatttggcaagagaaagaggactggatggtttcatatcaacctcaaagactcaatagagttaataaccatctgttggtatccactgggcaattgtcttttcacaataaactctgaaaactcagaacaaggaagattggtaaatgtcaatcataaaaaagtagaagttaagggacaaaggaattaagttgctggcataagaggaatcctcgtgccgaat